(19) United States
(12) Patent Application Publication
(10) Pub. No.: US 2023/0105984 A1
(43) Pub. Date: Apr. 6, 2023

(54) COMPOSITIONS AND METHODS FOR THE TREATMENT OF LIVER DISEASES AND DISORDERS

(71) Applicants: Nadine Daou, Cambridge, MA (US); Svetlana Marukian, Cambridge, MA (US); AXCELLA HEALTH INC., Cambridge, MA (US)

(72) Inventors: Nadine Daou, Medford, MA (US); William Comb, Melrose, MA (US); Alison Williams, Cambridge, MA (US); Svetlana Marukian, Watertown, MA (US); Michael Hamill, Wellesley, MA (US); Raffi Afeyan, Cambridge, MA (US); Sean Carroll, Cambridge, MA (US); Anthony Tramontin, Brooklyn, NY (US); Manu Chakravarthy, Newton, MA (US)

(73) Assignees: Svetlana Marukian, Cambridge, MA (US); Svetlana Marukian, Cambridge, MA (US); Nadine Daou, Cambridge, MA (US); Svetlana Marukian, Cambridge, MA (US)

(21) Appl. No.: 17/788,474

(22) PCT Filed: Dec. 23, 2020
(86) PCT No.: PCT/US2020/066903

§ 371 Date: Jun. 23, 2022

Related U.S. Application Data
(60) Provisional application No. 62/952,823, filed on Dec. 23, 2019.

(51) Int. Cl. A61K 31/198; A61K 31/194; A61K 31/221; A61K 31/46; A61K 31/4172; A61K 31/519; A61P 1/16

(52) U.S. Cl. CPC A61K 31/198; A61K 31/46; A61K 31/194; A61K 31/221; A61K 31/519; A61K 31/4172; A61P 1/16

(57) ABSTRACT

This disclosure provides combinations and methods for improving liver function, e.g., in a subject having a liver disease or disorder, or treating or preventing a liver disease or disorder, e.g., with hyperammonemia or muscle wasting in a subject.

### CROSS-REFERENCE TO RELATED APPLICATIONS

This application claims priority under 35 U.S.C. § 119(e) to U.S. Provisional Pat. Application Serial No. 62/952,823, the disclosures of which are hereby incorporated herein by reference in their entirety for all purposes.

### BACKGROUND

Non-alcoholic fatty liver disease (NAFLD) is a disease characterized by fatty deposits in the liver due to causes other than alcohol. NAFLD is the most prevalent liver disease in developed countries and affects close to 25% of the people in the United States. Non-alcoholic steatohepatitis (NASH) is the most severe form of NAFLD, which can lead to fibrosis, cirrhosis, chronic liver failure, and hepatocellular carcinoma (HCC).

Fatty liver disease and steatohepatitis associated with alcohol consumption are also significant health problems, and while the disease cause is different (exposure to ethanol), the impact on the liver, including accumulation of fatty deposits leading to fibrosis, cirrhosis, chronic liver failure, and hepatocellular carcinoma are the same.

Given the prevalence of liver diseases and disorders and the lack of approved therapies, the need exists for therapies that modulate the metabolic profile of subjects with liver diseases and disorders. Such agents can be used, e.g., for the treatment of NAFLD (e.g., NASH).

An estimated 600,000 individuals in the US suffer from cirrhosis and 14,000 of these patients are in end-stage disease and await liver transplant. Studies have suggested that up to 40-60% of cirrhosis patients have concomitant muscle wasting. The resultant frailty is a significant cause for functional decline, cirrhosis-related complications, hospitalizations, and mortality in patients with end-stage liver disease (ESLD). Liver transplant is the definitive cure for ESLD, but physical decline, independent of liver disease severity, is associated with increased risk of de-listing from transplant waitlists. Cirrhosis resulting from NASH is a growing population and expected to drive the majority of transplant recipients in coming decades.

An estimated 40-50% of cirrhosis patients exhibit cirrhotic sarcopenia. Cirrhotic sarcopenia is a frequent complication in cirrhosis that adversely impacts the survival and quality of life of patients. Cirrhotic sarcopenia is a systemic disease resulting from hyperammonemia due to a dysfunctional urea cycle in cirrhosis, in which the muscle detoxifies the ammonia, but at the expense of muscle mass. Sarcopenia lowers the survival, decreases the chances of receiving a transplant, and increases the risks of cirrhosis-related complications in cirrhosis patients. More recently, muscle quality (e.g., fat content) is associated with risk to hepatic encephalopathy and frailty associated with poor outcomes awaiting liver transplant. Inflammation has been appreciated to compound these disturbances and is clearly predictive of outcomes in ESLD.

The current standard of care for patients with cirrhosis, such as patients with ESLD or cirrhotic sarcopenia, includes lifestyle modifications, such as increased exercise and dietary interventions. Currently, there are no approved pharmacological interventions.

Given the lack of available therapies, there is still a need for agents, e.g., dietary compositions and therapeutics for treating liver diseases and disorders with hyperammonemia and sarcopenia (diminished muscle mass and/or function) especially in cirrhosis, cirrhotic sarcopenia, ESLD, hepatic insufficiency, or hepatic encephalopathy.

### SUMMARY OF THE INVENTION

The disclosure is directed in part to methods of treating a subject comprising administering to the subject a combination of a composition of amino acid entities (e.g., an Active Moiety) and a therapeutic agent. It has now been recognized that the unique, multifactorial, and endogenous action of compositions comprising amino acid entities designed to address liver diseases or disorders can work in combination (e.g., synergistically) with other therapeutic agents as described herein to more effectively address liver diseases or dysfunctions, such as NAFLD, NASH, AFLD, and ASH. Without wishing to be bound by theory, administration of the combination can improve the therapeutic window, e.g., may result in one or both of greater efficacy than administration of a monotherapy or administration of a lower dosage of one or both of the composition comprising amino acid entities (e.g., an Active Moiety) or the therapeutic agent as described herein. The present methods further provide for reduction of adverse events, e.g., through balancing metabolism through the multifactorial effects of the compositions, while promoting targeted effects of the therapeutic agent.

The composition of amino acid entities (e.g., an Active Moiety) can comprise at least five amino acid entities (e.g., six or seven amino acid entities). In some embodiments, the composition comprises two or more amino acid entities (e.g., a N-acetylcysteine (NAC) entity and a carnitine (CAR) entity, and optionally one or more additional amino acid entities).

In some embodiments, the therapeutic agent is chosen from the following categories: (i) a hepatic fat accumulation inhibitor; or (ii) one or both of an oxidative stress or hepatic inflammation inhibitor. In an embodiment, a plurality of therapeutic agents is selected from, e.g., one or both of categories (i)-(ii).

In an embodiment, the composition of amino acid entities (e.g., an Active Moiety) and a therapeutic agent are provided in a single dosage form. In an embodiment the composition of amino acid entities (e.g., an Active Moiety) and a therapeutic agent are provided as separate single dosage forms for co-administration or coordinated administration.

In some embodiments, the combination is useful for improving liver function in a subject, e.g., a subject with a liver disease or disorder (e.g., NAFLD (e.g., NASH or NAFL) or AFLD (e.g., ASH)). The combination can be used in a method of treating (e.g., reversing, reducing, ameliorating, or preventing) a liver disease or disorder in a subject in need thereof (e.g., a human).

The disclosure is also directed in part to a composition (e.g., an Active Moiety) including amino acid entities that is useful for improving one, two, three, or more (e.g., all) of liver function, hyperammonemia, inflammation, and muscle mass, quality, or function in a subject, e.g., a subject with a liver disease or disorder with one or both of hyperammonemia or muscle wasting. The composition can be used in a method of treating (e.g., reversing, reducing, ameliorating, or preventing) complications associated with chronic liver disease or disorder with one or both of hyperammonemia or muscle wasting (e.g., cirrhosis, e.g., cirrhotic sarcopenia, End Stage Liver Disease (ESLD), hepatic insufficiency, or hepatic encephalopathy) in a subject in need thereof (e.g., a human) experiencing associated complications including ascites, infections, varices, or encephalopathy.

Accordingly, in one aspect, the invention features a combination comprising a composition (e.g., an Active Moiety) and a therapeutic agent, wherein the composition comprises:

a) N-acetylcysteine (NAC) or a salt thereof or a dipeptide or salt thereof, comprising NAC; and
(b) L-carnitine or a salt thereof, or a dipeptide or a salt thereof, comprising L-carnitine; or acetyl-L-carnitine (ALCAR) or a salt thereof or a dipeptide or salt thereof, or a tripeptide or salt thereof, comprising ALCAR; and
the therapeutic agent is chosen from one or both of the following categories:
   (i) a hepatic fat accumulation inhibitor; or
   (ii) one or both of an oxidative stress or hepatic inflammation inhibitor.

In another aspect, the invention features a combination comprising a composition (e.g., an Active Moiety) and a therapeutic agent, wherein the composition comprises:
a) a leucine amino acid entity chosen from:
   i) L-leucine or a salt thereof,
   ii) a dipeptide or salt thereof, or tripeptide or salt thereof, comprising L-leucine, or
   iii) β-hydroxy-β-methylbutyrate (HMB) or a salt thereof;
b) a arginine amino acid entity chosen from:
   i) L-arginine or a salt thereof,
   ii) a dipeptide or salt thereof, or tripeptide or salt thereof, comprising L-arginine,
   iii) ornithine or a salt thereof,
   iv) a dipeptide or salt thereof, or tripeptide or salt thereof, comprising ornithine,
   v) creatine or a salt thereof, or
   vi) a dipeptide or salt thereof, or tripeptide or salt thereof, comprising creatine;
c) L-glutamine or a salt thereof or a dipeptide or salt thereof, or tripeptide or salt thereof, comprising L-glutamine;
d) N-acetylcysteine (NAC) or a salt thereof or a dipeptide or salt thereof, comprising NAC; and
e) one or both of
   i) L-serine or a salt thereof or a dipeptide or salt thereof, or tripeptide or salt thereof, comprising L-serine; or
   ii) L-carnitine or a salt thereof or a dipeptide or salt thereof, or tripeptide or salt thereof, comprising L-carnitine,

wherein optionally one or both of:
the wt. % of the serine amino acid entity is at least 8 wt. % of the total weight of amino acid entities in the composition or total components in the composition; or
the wt. % of the carnitine entity is at least 2 wt. % of the total weight of amino acid entities in the composition or total components in the composition; and
wherein the therapeutic agent is chosen from one or both of the following categories:
(i) a hepatic fat accumulation inhibitor; or
(ii) one or both of an oxidative stress or hepatic inflammation inhibitor.

In another aspect, the invention features a combination (e.g., one or more compositions or dosage forms) comprising a composition and a therapeutic agent, wherein the composition comprises:
a) a leucine amino acid entity,
b) an isoleucine amino acid entity,
c) a arginine amino acid entity,
d) a N-acetylcysteine (NAC) entity; and
e) a carnitine entity; and
the therapeutic agent is chosen from one or both of the following categories:
   (i) a hepatic fat accumulation inhibitor; or
   (ii) one or both of an oxidative stress or hepatic inflammation inhibitor,
wherein the total wt. % of (a)-(e) is greater than the total wt. % of other amino acid entities in the composition (e.g., in dry form); and
wherein optionally the wt. % of the carnitine entity is at least 2 wt. % of the amino acid entites or the total components in the composition (e.g., in dry form).

Without wishing to be bound by theory, the invention is directed in part to the discovery that NAC is effective to reduce liver inflammation and that CAR is effective to improve (e.g., reduce) lipid levels in liver. The disclosure teaches, in part, the unexpected synergistic effects of NAC and CAR when combined with a therapeutic agent as described herein, and/or with additional amino acid entities that improve liver function. The disclosure teaches, in part, the unexpected synergistic effects of NAC and CAR when combined with a therapeutic agent as described herein and/or with additional amino acid entities in the treatment of NASH, NAFLD, hepatic lipid accumulation, and/or hepatic inflammation.

In another aspect, the invention features a composition (e.g., an Active Moiety) comprising:
a) N-acetylcysteine (NAC) or a salt thereof or a dipeptide or salt thereof, comprising NAC; and
b) L-carnitine or a salt thereof, or a dipeptide or a salt thereof, comprising L-carnitine; or acetyl-L-carnitine (ALCAR) or a salt thereof or a dipeptide or salt thereof, or a tripeptide or salt thereof, comprising ALCAR.

In some embodiments, the total weight (wt.) % of (a) and (b) is greater than 50% of the total wt. of amino acid entities. In some embodiments, the wt. % of (b) is at least 2% and up to 10% of the total wt. of amino acid entities in the composition.

In another aspect, the invention features a composition comprising, consisting of, or consisting essentially of:
a) a Branched Chain Amino Acid (BCAA) entity chosen from a leucine amino acid entity, an isoleucine amino acid entity, a valine amino acid entity, or a combination of two or three BCAA entities;
b) a Urea Cycle Amino Acid (UCAA) entity chosen from an ornithine amino acid entity, an aspartate amino acid entity, or a combination of two UCAA entities;
c) an essential amino acid (EAA) entity chosen from a histidine amino acid entity, a lysine amino acid entity, or a threonine amino acid entity or a combination of two or three EAA entities; and
d) one or two of:
   i) N-acetylcysteine (NAC) or a salt thereof, or a dipeptide or a salt thereof, comprising NAC; or
   ii) L-carnitine or a salt thereof, or a dipeptide or a salt thereof, comprising L-carnitine; or acetyl-L-carnitine (ALCAR) or a salt thereof or a dipeptide or salt thereof, or a tripeptide or salt thereof, comprising ALCAR;

wherein at least one amino acid entity (e.g., two, three, four, five, six, seven, or eight amino acid entities) of (a)-(c) is not provided as a peptide of more than 20 amino acid residues in length.

Without wishing to be bound by theory, the invention is directed in part to the discovery that NAC is effective to reduce inflammation in muscle and liver and that CAR is effective to improve (e.g., reduce) lipid levels in muscle and liver. The disclosure teaches, in part, the unexpected synergistic effects of NAC and CAR in the treatment of cirrhosis and/or hepatic encephalopathy and symptoms related thereto. The disclosure teaches, in part, the unexpected synergistic effects of NAC, CAR, and additional amino acid entities in the treatment of cirrhosis and/or hepatic encephalopathy and symptoms related thereto.

The hepatic fat accumulation inhibitor can be chosen from a an acetyl-CoA carboxylase inhibitor, an FXR agonist, or a combination thereof. In certain embodiments, the acetyl-CoA carboxylase inhibitor is chosen from GS-0976 (e.g., Firsocostat), PF-05221304, gemcabene, or a salt or a derivative of any of the foregoing. In certain embodiments, the FXR agonist is chosen from GS-9674 (e.g., Cilofexor), LJN-452 (Tropifexor), obeticholic acid (OCA, e.g., Ocaliva), EDP-305, EYP001, or a salt or a derivative of any of the foregoing.

One or both of an oxidative stress or hepatic inflammation inhibitor can be chosen from an ASK1 inhibitor. In certain embodiments, the ASK1 inhibitor is selonsertib or a salt or a derivative thereof. In some embodiments, one or both of the composition (e.g., Active Moiety) or the therapeutic agent is formulated with a pharmaceutically acceptable carrier.

In some embodiments, the composition (e.g., Active Moiety) is a dietary composition.

In another aspect, the invention features a method for improving liver function, comprising administering to a subject in need thereof an effective amount of the combination of any of the aspects or embodiments disclosed herein, thereby improving liver function in the subject. Administering an effective amount of the combination includes administering a unit dose that contains both the composition and the therapeutic agent as well as administering each of the composition and the therapeutic agent separately in any sequence, timing, and frequency; provided that the patient receives both actives at an amount and dose to provide the benefits of the invention, such as an improved therapeutic window, lower toxicity, reduction in adverse events. In some embodiments, a daily dose of an oral therapeutic agent can be given in coformulation with a daily dose of the composition of amino acid entities. In some embodiments, a daily dose of an oral therapeutic agent can be given in coformulation once per day with a composition of amino acid entities once per day and the composition of amino acid entities given alone once per day. In some embodiments, an infusion of a therapeutic agent (e.g., a biologic, e.g. peptide, polypeptide, or antibody) can be given once every week or every 2 weeks and the composition of amino acid entities given twice per day.

In another aspect, the invention features a method for treating a symptom chosen from one, two, three, four, five, six, seven, eight, nine, ten, or more (e.g., all) of: decreased fat metabolism, hepatocyte apoptosis, hepatocyte ballooning, inflammation of adipose tissue, inflammation of hepatic tissue, fibrosis, liver injury, steatosis, glucose tolerance, insulin resistance, or oxidative stress, comprising administering to a subject in need thereof an effective amount of the combination of any of the aspects or embodiments disclosed herein, thereby treating the symptom in the subject.

In another aspect, the invention features a method for treating a liver disease or disorder, comprising administering to a subject in need thereof an effective amount of the combination of any of the aspects or embodiments disclosed herein, thereby treating the liver disease or disorder in the subject.

In another aspect, the invention features a method of improving or treating a symptom selected from one, two, three, four, five, six, seven, eight, nine, 10, 11, 12, 13, 14, 15, 16, 17, or more (e.g., all) of hyperammonemia, ascites or complications associated with ascites, variceal bleeding, infection, hepatic encephalopathy, ammonia toxicity, hepatic insufficiency, decreased urea synthesis, inflammation of hepatic tissue, fibrosis, cirrhosis, muscle wasting, muscle catabolism, muscle atrophy, hypoalbuminemia, malnutrition, frailty, or coagulopathy, comprising administering to a subject in need thereof an effective amount of a composition (e.g., an Active Moiety) of any of the aspects or embodiments disclosed herein, thereby improving or treating the symptom in the subject.

In another aspect, the invention features a method for treating or preventing a liver disease or disorder characterized by one or both of hyperammonemia or muscle wasting, comprising administering to a subject in need thereof an effective amount of a composition (e.g., an Active Moiety) of any of the aspects or embodiments disclosed herein, thereby treating the liver disease or disorder or muscle wasting in the subject.

In some aspects, the present disclosure provides diagnostic or therapeutic kits that include the combinations or compositions described herein and instructions for use.

Additional features of any of the aforesaid methods or compositions include one or more of the following enumerated embodiments.

Those skilled in the art will recognize, or be able to ascertain using no more than routine experimentation, many equivalents to the specific embodiments of the invention described herein. Such equivalents are intended to be encompassed by the following enumerated embodiments.

All publications, patent applications, patents, and other references (e.g., sequence database reference numbers) mentioned herein are incorporated by reference in their entirety.

### Enumerated Embodiments

1. A combination (e.g., one or more compositions or dosage forms) comprising a composition and a therapeutic agent, wherein the composition comprises:
   (a) N-acetylcysteine (NAC) or a salt thereof, or a dipeptide or a salt thereof, comprising NAC;
   (b) L-carnitine or a salt thereof, or a dipeptide or a salt thereof, comprising L-carnitine; or acetyl-L-carnitine (ALCAR) or a salt thereof or a dipeptide or salt thereof, or a tripeptide or salt thereof, comprising ALCAR; and
   the therapeutic agent is chosen from one or both of the following categories:
      (i) a hepatic fat accumulation inhibitor; or
      (ii) one or both of an oxidative stress or hepatic inflammation inhibitor.
2. The combination of embodiment 1, further comprising a leucine amino acid entity chosen from:
   i) L-leucine or a salt thereof,
   ii) a dipeptide or salt thereof, or tripeptide or salt thereof, comprising L-leucine, or
   iii) β-hydroxy-β-methylbutyrate (HMB) or a salt thereof.
3. The combination of either of embodiments 1 or 2, further comprising an arginine amino acid entity chosen from:
   i) L-arginine or a salt thereof,
   ii) a dipeptide or salt thereof, or tripeptide or salt thereof, comprising L-arginine,
   iii) ornithine or a salt thereof,
   iv) a dipeptide or salt thereof, or tripeptide or salt thereof, comprising ornithine,
   v) creatine or a salt thereof, or
   vi) a dipeptide or salt thereof, or tripeptide or salt thereof, comprising creatine.
4. The combination of any of embodiments 1-3, further comprising L-glutamine or a salt thereof or a dipeptide or salt thereof, or tripeptide or salt thereof, comprising L-glutamine.
5. The combination of any of embodiments 1-4, further comprising L-serine or a salt thereof or a dipeptide or salt thereof, or tripeptide or salt thereof, comprising L-serine.
6. The combination of embodiment 5, wherein the wt. % of the serine amino acid entity is at least 8 wt. % of the total amino acid entity components or total components in the composition.
7. The combination of any of embodiments 1-6, wherein the wt. % of the carnitine entity is at least 2 wt. % of the total amino acid entity components or total components in the composition.
8. A combination (e.g., one or more compositions or dosage forms) comprising a composition and a therapeutic agent, wherein the composition comprises:
   a) a leucine amino acid entity chosen from:
      i) L-leucine or a salt thereof,
      ii) a dipeptide or salt thereof, or tripeptide or salt thereof, comprising L-leucine, or
      iii) β-hydroxy-β-methylbutyrate (HMB) or a salt thereof;
   b) a arginine amino acid entity chosen from:
      i) L-arginine or a salt thereof,
      ii) a dipeptide or salt thereof, or tripeptide or salt thereof, comprising L-arginine,
      iii) ornithine or a salt thereof,
      iv) a dipeptide or salt thereof, or tripeptide or salt thereof, comprising ornithine,
      v) creatine or a salt thereof, or
      vi) a dipeptide or salt thereof, or tripeptide or salt thereof, comprising creatine;
   c) L-glutamine or a salt thereof or a dipeptide or salt thereof, or tripeptide or salt thereof, comprising L-glutamine;
   d) N-acetylcysteine (NAC) or a salt thereof or a dipeptide or salt thereof, comprising NAC;
   e) one or both of
      i) L-serine or a salt thereof or a dipeptide or salt thereof, or tripeptide or salt thereof, comprising L-serine; or
      ii) L-carnitine or a salt thereof or a dipeptide or salt thereof, or tripeptide or salt thereof, comprising L-carnitine; and the therapeutic agent is chosen from one or both of the following categories:
      (i) a hepatic fat accumulation inhibitor; or
      (ii) one or both of an oxidative stress or hepatic inflammation inhibitor,
   wherein optionally one or both of:
   the wt. % of the serine amino acid entity is at least 8 wt. % of the total amino acid entity components or total components in the composition; or
   the wt. % of the carnitine entity is at least 2 wt. % of the total amino acid entity components or total components in the composition.
9. The combination of any of embodiments 1-8, wherein the composition further comprises: f) L-isoleucine or a salt thereof or a dipeptide or salt thereof, or a tripeptide or salt thereof, comprising L-isoleucine.
10. The combination of any of embodiments 1-9, wherein the composition comprises:
   a) the leucine amino acid entity is L-leucine or a salt thereof;
   b) the arginine amino acid entity is L-arginine or a salt thereof;
   c) L-glutamine or a salt thereof;
   d) NAC or a salt thereof;
   e) L-serine or a salt thereof
   f) L-carnitine or a salt thereof; and
   g) L-isoleucine or a salt thereof.
11. A combination (e.g., one or more compositions or dosage forms) comprising a composition and a therapeutic agent, wherein the composition comprises:
   a) a leucine amino acid entity,
   b) an isoleucine amino acid entity,
   c) a arginine amino acid entity,
   d) a N-acetylcysteine (NAC) entity; and
   e) a carnitine entity; and the therapeutic agent is chosen from one or both of the following categories:
      (i) a hepatic fat accumulation inhibitor; or
      (ii) one or both of an oxidative stress or hepatic inflammation inhibitor,
   wherein the total wt. % of (a)-(e) is greater than the total wt. % of other amino acid entities in the composition (e.g., in dry form); and
   wherein optionally the wt. % of the carnitine entity is at least 2 wt. % of the amino acid entites or the total components in the composition (e.g., in dry form).
12. The combination of embodiment 11, wherein the composition further comprises: (f) one or both of a glutamine amino acid entity or a serine amino acid entity.
13. The combination of any preceding embodiment, wherein the therapeutic agent is a hepatic fat accumulation inhibitor.
14. The combination of any preceding embodiment, wherein the therapeutic agent is one or both of an oxidative stress or hepatic inflammation inhibitor.
15. The combination of any preceding embodiment, wherein the therapeutic agent comprises a hepatic fat accumulation inhibitor and one or both of an oxidative stress or hepatic inflammation inhibitor.
16. The combination of either of embodiments 14 or 15, wherein the therapeutic agent is or comprises an oxidative stress inhibitor.
17. The combination of either of embodiments 14 or 15, wherein the therapeutic agent is or comprises a hepatic inflammation inhibitor.
18. The combination of either of embodiments 14 or 15, wherein the therapeutic agent is or comprises both an oxidative stress inhibitor and a hepatic inflammation inhibitor.
19. The combination of any preceding embodiment, wherein the hepatic fat accumulation inhibitor is chosen from an acetyl-CoA carboxylase inhibitor, an FXR agonist, or a combination thereof.
20. The combination of embodiment 19, wherein the hepatic fat accumulation inhibitor is an acetyl-CoA carboxylase inhibitor.
21. The combination of embodiment 19, wherein the hepatic fat accumulation inhibitor is an FXR agonist.
22. The combination of embodiment 19, wherein the hepatic fat accumulation inhibitor is or comprises an FXR agonist and an acetyl-CoA carboxylase inhibitor.
23. The combination of any of embodiments 19, 20, or 22, wherein the acetyl-CoA carboxylase inhibitor is chosen from GS-0976 (e.g., Firsocostat), PF-05221304, gemcabene, or a salt or a derivative of any of the foregoing.
24. The combination of any of embodiments 19 or 21-23, wherein the FXR agonist is chosen from GS-9674 (e.g., Cilofexor), LJN-452 (Tropifexor), obeticholic acid (OCA, e.g., Ocaliva), EDP-305, EYP001, or a salt or a derivative of any of the foregoing.
25. The combination of any preceding embodiment, wherein the one or both of an oxidative stress or hepatic inflammation inhibitor is an ASK1 inhibitor.
26. The combination of embodiment 25, wherein the ASK1 inhibitor is selonsertib or a salt or a derivative thereof.
27. A composition comprising:
   (a) N-acetylcysteine (NAC) or a salt thereof, or a dipeptide or a salt thereof, comprising NAC; and
   (b) L-carnitine or a salt thereof, or a dipeptide or a salt thereof, comprising L-carnitine; or acetyl-L-carnitine (ALCAR) or a salt thereof or a dipeptide or salt thereof, or a tripeptide or salt thereof, comprising ALCAR,
   wherein the total weight (wt.) % of (a) and (b) is greater than 50% of the total wt. of amino acid entities, or
   wherein the wt. % of (b) is at least 2% and up to 10% of the total wt. of amino acid entities.
28. The composition of embodiment 27 further comprising a Branched Chain Amino Acid (BCAA) entity chosen from a leucine amino acid entity, an isoleucine amino acid entity, a valine amino acid entity, or a combination of two or three BCAA entities.
29. The composition of either embodiment 27 or 28, further comprising a Urea Cycle Amino Acid (UCAA) entity chosen from an ornithine amino acid entity chosen from L-ornithine, ornithine α-ketoglutarate, ornithine HCl, citrulline, or a combination thereof; an aspartate amino acid entity; or a combination of two UCAA entities.
30. The composition of any of embodiments 27-29, further comprising an essential amino acid (EAA) entity chosen from a histidine amino acid entity, a lysine amino acid entity, or a threonine amino acid entity or a combination of two or three EAA entities.
31. The composition of any of embodiments 28-30, wherein at least one of the BCAA, UCAA, or EAA is not provided as a peptide of more than 20 amino acid residues in length.
32. The composition of any of embodiments 27-31, wherein the total weight (wt.) % of (a), (b), and BCAA, UCAA, and EAA if present, is greater than the total wt. % of non-amino acid entity protein components or other amino acid entities in the compositionin the composition on a dry weight basis.
33. The composition of any of embodiments 27-32, wherein two or more (e.g., all) of phenylalanine, tyrosine, or glutamine is absent from the composition, or if present, is present at less than 1 wt. % of the total wt. of the composition on a dry weight basis.
34. A composition comprising:
   a) a Branched Chain Amino Acid (BCAA) entity chosen from a leucine amino acid entity, an isoleucine amino acid entity, a valine amino acid entity, or a combination of two or three BCAA entities;
   b) a Urea Cycle Amino Acid (UCAA) entity chosen from an ornithine amino acid entity chosen from L-ornithine, ornithine α-ketoglutarate, ornithine HCl, citrulline, or a combination thereof; an aspartate amino acid entity; or a combination of two UCAA entities; and
   c) an essential amino acid (EAA) entity chosen from a histidine amino acid entity, a lysine amino acid entity, or a threonine amino acid entity or a combination of two or three EAA entities; and
   d) one or two of:
      i) N-acetylcysteine (NAC) or a salt thereof, or a dipeptide or a salt thereof, comprising NAC; or
      ii) L-carnitine or a salt thereof, or a dipeptide or a salt thereof, comprising L-carnitine; or acetyl-L-carnitine (ALCAR) or a salt thereof or a dipeptide or salt thereof, or a tripeptide or salt thereof, comprising ALCAR;
   wherein:
   i) at least one amino acid entity of (a)-(c) is not provided as a peptide of more than 20 amino acid residues in length;
   ii) the total weight (wt.) % of (a)-(d) is greater than the total wt. % of non-amino acid protein components or other amino acid entity components in the composition on a dry weight basis; and
   iii) two or more (e.g., all) of phenylalanine, tyrosine, or glutamine is absent from the composition, or if present, is present at less than 1 wt. % of the total wt. of the composition on a dry weight basis.
35. The combination or composition of any preceding embodiment, wherein two, three, four, five, six, seven, or eight amino acid entities of (a)-(b); (a)-(b) and the BCAA, UCAA, and/or EAA; (a)-(b) and the leucine amino acid entity, arginine amino acid entity, L-glutamine (or a salt thereof or a dipeptide or salt thereof, or tripeptide or salt thereof, comprising L-glutamine), and/or L-serine (or a salt thereof or a dipeptide or salt thereof, or tripeptide or salt thereof, comprising L-serine); (a)-(c); or (a)-(d) is not provided as a peptide of more than 20 amino acid residues in length.
36. The combination or composition of embodimentany preceding embodiment, wherein the composition does not comprise a peptide of more than 20 amino acid residues in length, or if a peptide of more than 20 amino acid residues in length is present, the peptide is present at less than 10 wt. % of the total wt. of amino acid entities in the composition (in dry form).
37. The combination or composition of any embodiment preceding embodiment, wherein two, three, four, five, six, seven, or eight amino acid entities in (a)-(b); (a)-(b) and the BCAA, UCAA, and/or EAA; (a)-(b) and the leucine amino acid entity, arginine amino acid entity, L-glutamine (or a salt thereof or a dipeptide or salt thereof, or tripeptide or salt thereof, comprising L-glutamine), and/or L-serine (or a salt thereof or a dipeptide or salt thereof, or tripeptide or salt thereof, comprising L-serine); (a)-(c); or (a)-(d) are in one or both of free amino acid form or salt amino acid form in the composition (in dry form).
38. The combination or composition of embodiment 37, wherein at least 35 wt. % of the total wt. of the composition (in dry form) is two, three, four, five, six, seven, eight, nine, ten, or more (e.g., all) amino acid entities in (a)-(b); (a)-(b) and the BCAA, UCAA, and/or EAA; (a)-(b) and the leucine amino acid entity, arginine amino acid entity, L-glutamine (or a salt thereof or a dipeptide or salt thereof, or tripeptide or salt thereof, comprising L-glutamine), and/or L-serine(or a salt thereof or a dipeptide or salt thereof, or tripeptide or salt thereof, comprising L-serine); (a)-(c); or (a)-(d) in one or both of free amino acid form or salt amino acid form.
39. The composition of any of embodiments 28-38, wherein:
   i) the wt. % of the BCAA entities is at least 37 wt. % of the total wt. of amino acid entities in the composition (in dry form);
   ii) the wt. % of the UCAA entities is at least 25 wt. % of the total wt. of amino acid entities in the composition (in dry form); or
   iii) the wt. % of the EAA entities is at least 16 wt. % of the total wt. of amino acid entities in the composition (in dry form).
40. The composition of any of embodiments 30-39, wherein a wt. ratio of the BCAA entity or BCAA entities : the UCAA entity or UCAA entities : the EAA entity or EAA entities in 20+/- 15% : 15 +/- 15%: 9+/- 15%, where the ratios are determined based on an equivalent amount of each amino acid in free form.
41. The composition of any of embodiments 34-40, wherein three, four, five, six, seven, or eight amino acid entities in (a)-(c) is selected from Table 1.
42. The composition of any of embodiments 34-41, wherein the composition comprises:
   a) the leucine amino acid entity is chosen from:
      i) L-leucine or a salt thereof,
      ii) a dipeptide or salt thereof, or tripeptide or salt thereof, comprising L-leucine, or
      iii) β-hydroxy-β-methylbutyrate (HMB) or a salt thereof;
   b) one or both of:
      i) the ornithine amino acid entity is L-ornithine or a salt thereof or a dipeptide or salt thereof, or tripeptide or salt thereof, comprising L-ornithine; or
      ii) the aspartate amino acid entity is L-aspartate or a salt thereof or a dipeptide or salt thereof, or tripeptide or salt thereof, comprising L-aspartate;
   c) the EAA entity is chosen from:
      i) L-histidine or a salt thereof,
      ii) a dipeptide or salt thereof, or tripeptide or salt thereof, comprising L-histidine,
      iii) L-lysine or a salt thereof,
      iv) a dipeptide or salt thereof, or tripeptide or salt thereof, comprising L-lysine,
      v) L-threonine or a salt thereof, or
      vi) a dipeptide or salt thereof, or tripeptide or salt thereof, comprising L-threonine; and
   d) one or two of:
      i) N-acetylcysteine (NAC) or a salt thereof, or a dipeptide or a salt thereof, comprising NAC; or
      ii) L-carnitine or a salt thereof, or a dipeptide or a salt thereof, comprising L-carnitine; or acetyl-L-carnitine (ALCAR) or a salt thereof or a dipeptide or salt thereof, or a tripeptide or salt thereof, comprising ALCAR.
43. The composition of embodiment 42, wherein the composition further comprises one or both of an isoleucine amino acid entity or a valine amino acid entity.
44. The composition of embodiment 43, wherein the isoleucine amino acid-entity is L-isoleucine or a salt thereof or a dipeptide or salt thereof, or tripeptide or salt thereof, comprising L-isoleucine.
45. The composition of embodiment 43, wherein the valine amino acid entity is L-valine or a salt thereof or a dipeptide or salt thereof, or tripeptide or salt thereof, comprising L-valine.
46. The composition of either of embodiments 44 or 45, wherein a wt. ratio of the leucine amino acid entity : the isoleucine amino acid entity : the valine amino acid entity : the ornithine amino acid entity : the aspartate amino acid entity : the histidine amino acid entity : the threonine amino acid entity : the lysine amino acid entity is 8+/- 20% : 4+/- 20% : 8 +/- 20% : 7.5+/- 20% : 7.5+/- 20% : 3+/- 20% : 3+/- 20% : 3+/- 20%, where the ratios are determined based on an equivalent amount of each amino acid in free form.
47. The composition of any of embodiments 34-46, wherein the composition comprises: L-leucine or a salt thereof, L-isoleucine or a salt thereof, L-valine or a salt thereof, L-ornithine or a salt thereof, L-aspartate or a salt thereof, L-histidine or a salt thereof, L-threonine or a salt thereof, L-lysine or a salt thereof, and one or two of:
   i) NAC or a salt thereof; or
   ii) L-carnitine or a salt thereof, or ALCAR or a salt thereof.
48. The combination or composition of any preceding embodiment, wherein the composition is formulated with a pharmaceutically acceptable carrier.
49. The combination of any of embodiments 1-26 or 48, wherein the therapeutic agent is formulated with a pharmaceutically acceptable carrier.
50. The combination or composition of any preceding embodiment, wherein the composition is formulated as a dietary composition.
51. A method of improving one or more of liver function, hyperammonemia, muscle mass, or muscle function, the method comprises administering to a subject with cirrhosis an effective amount of the composition of any of embodiments 27-50.
52. A method of improving or treating a symptom selected from the group consisting of hyperammonemia, ascites or complications associated with ascites, variceal bleeding, infection, hepatic encephalopathy, ammonia toxicity, hepatic insufficiency, decreased urea synthesis, inflammation of hepatic tissue, fibrosis, cirrhosis, muscle wasting, muscle catabolism, muscle atrophy, hypoalbuminemia, malnutrition, frailty, and coagulopathy, comprising administering to a subject in need thereof an effective amount of the composition of any of embodiments 27-50, thereby improving or treating the symptom in the subject.
53. A method of treating or preventing a liver disease or disorder with one or both of hyperammonemia or muscle wasting, wherein the method comprises administering to a subjectin need thereof an effective amount of a composition of any of embodiments 25-48, thereby treating or preventing the liver disease or disorder.
54. The method of embodiment 52 or 53, wherein the subject has cirrhosis.
55. The method of any of embodiments 51-53, wherein the subject has cirrhotic sarcopenia, hepatic insufficiency, End Stage Liver Disease, hepatic encephalopathy, or a combination thereof.
56. The method of any of embodiments 51-55, wherein administration of the composition results in at least one of the following: a) increased level of BCAAs; b) decreased level of aromatic amino acids (AAAs); c) decreased level of ammonia; d) increased level of protein, e.g., increased protein synthesis; e) increased activation of mTORC1; f) decreased level of myostatin; g) decreased level of creatinine; h) increased level of albumin; i) decreased level of bilirubin; j) increased Fischer’s ratio (e.g., increased level of BCAAs relative to the level of AAAs); or k) an increased level of valine relative to a level of phenylalanine.
57. The method of any of embodiments 51-56, wherein the composition is administered with a carbohydrate supplement, e.g., when administered in the night, late evening, or before bedtime.
58. A method for treating a symptom chosen from one, two, three, four, five, six, seven, eight, nine, ten, or more (e.g., all) of: decreased fat metabolism, hepatocyte apoptosis, hepatocyte ballooning, inflammation of adipose tissue, inflammation of hepatic tissue, fibrosis, liver injury, steatosis, glucose tolerance, insulin resistance, or oxidative stress, comprising administering to a subject in need thereof an effective amount of a combination of any of embodiments 1-26 or 48-50, thereby treating the symptom in the subject.
59. A method for treating a liver disease or disorder, comprising administering to a subject in need thereof an effective amount of a combination of any of embodiments 1-26 or 48-50,thereby treating the liver disease or disorder in the subject.
60. A method for improving liver function, comprising administering to a subject in need thereof an effective amount of a combination of any of embodiments 1-26 or 48-50, thereby improving liver function in the subject.
61. The method of any of embodiments 58-60, wherein the subject has a fatty liver disease or disorder, e.g., the fatty liver disease or disorder is chosen from: non-alcoholic fatty liver disease (NAFLD) or alcoholic fatty liver disease (AFLD).
62. The method of embodiment 61, wherein the NAFLD is chosen from: non-alcoholic steatohepatitis (NASH) or non-alcoholic fatty liver (NAFL).
63. The method of embodiment 61, wherein the NAFLD is pediatric NAFLD.
64. The method of embodiment 61, wherein the AFLD is alcoholic steatohepatitis (ASH).
65. The method of any of embodiments 58-64, wherein the subject has one, two, three, four, five, or more (e.g., all) of cirrhosis, fibrosis, hepatocarcinoma, steatosis, an increased risk of liver failure, or an increased risk of death.
66. The method of any of embodiments 58-65, wherein the subject has one, two, three, four, five, six, or more (e.g., all) of type 2 diabetes, metabolic syndrome, a high BMI, obesity, gut leakiness, gut dysbiosis, or gut microbiome disturbance.
67. The method of any of embodiments 58-66, wherein administration of the combination results in one, two, three, four, five, six, seven, eight, or more (e.g., all) of: decreasing or preventing liver fibrosis, decreasing or preventing liver injury, decreasing or preventing hepatocyte inflammation, improving glucose tolerance; improving insulin resistance, decreasing or preventing steatosis, decreasing or preventing hepatocyte ballooning, increasing liver fatty acid oxidation, or improving gut function.
68. The method of any of embodiments 58-67, wherein administering the combination comprises administering the composition and the therapeutic agent together, e.g., as a unit dose that contains both the composition and the therapeutic agent.
69. The method of any of embodiments 58-67, wherein administering the combination comprises administering the composition and the therapeutic agent separately, e.g., as unit dose containing the composition and a unit dose containing the therapeutic agent.
70. The method of either embodiment 68 or 69, wherein the composition and the therapeutic are administered concurrently.
71. The method of embodiment 69, wherein the composition and the therapeutic agent are administered sequentially, e.g., the composition is administered and then the therapeutic agent is administered, or the therapeutic agent is administered and then the composition is administered.
72. The method of embodiment 71, wherein a time interval separates the administration of the composition and the administration of the therapeutic agent, e.g., a time interval of seconds, minutes, hours, days, or weeks.

### DETAILED DESCRIPTION

Described herein, in part, is a combination comprising a composition comprising amino acid entities (e.g., an Active Moiety) and one or more therapeutic agents and methods of improving liver function by administering an effective amount of the combination. In some embodiments, the composition comprises at least two, three, four, five, six, or seven amino acid entities. In some embodiments, the combination comprises a therapeutic agent chosen from one or both of the following categories: (i) a hepatic fat accumulation inhibitor; or (ii) one or both of an oxidative stress or hepatic inflammation inhibitor. The combination may be administered in an effective amount to treat or prevent a liver disease or disorder as described herein in a subject in need thereof. Administering an effective amount of the combination includes administering a unit dose that contains both the composition and the therapeutic agent as well as administering each of the composition and the therapeutic agent separately in any sequence, timing, and frequency; provided that the patient receives both actives at an amount and dose to provide the benefits of the invention, such as an improved therapeutic window, lower toxicity, reduction in adverse events. In some embodiments, a daily dose of an oral therapeutic agent can be given in coformulation with a daily dose of the composition of amino acid entities. In some embodiments, a daily dose of an oral therapeutic agent can be given in coformulation once per day with a composition of amino acid entities once per day and the composition of amino acid entities given alone once per day. In some embodiments, an infusion of a therapeutic agent (e.g., a biologic, e.g. peptide, polypeptide, or antibody) can be given once every week or every 2 weeks and the composition of amino acid entities given twice per day.

It has not been recognized that the unique, multifactorial, and endogenous action of such compositions comprising amino acid entities (e.g., an Active Moiety) designed to address liver diseases and disorders can work in combination (e.g., synergistically) with therapeutic agents as described herein. Without wishing to be bound by theory, in some embodiments, administration of the combination results in one or both of greater efficacy than a monotherapy (e.g., administration of the composition comprising amino acid entities (e.g., an Active Moiety) or the therapeutic agent alone) or lower doses of the composition comprising amino acid entities (e.g., an Active Moiety) or the therapeutic agent (e.g., thereby resulting in reduced side effects). In some embodiments, administration of composition targets multiple axes of a disease (e.g., a liver disease or disorder) relative to a monotherapy.

The combination disclosed herein allows for multi-pathway beneficial effects on liver function to optimize modulation of signaling pathways, such as one, two, three, or more (e.g., all) of inflammation, lipid metabolism, fibrosis, or glucose metabolism. In some embodiments, the combination disclosed herein results in one, two, three, four, five, six, seven, or more (e.g., all) decreased hepatic fat accumulation, increased mitochondrial function, increased insulin sensitivity, increased glucose tolerance, decreased hepatic steatosis, decreased inflammation, decreased fibrosis, or increased fatty acid oxidation. For example, the amino acid entities and relative amounts of the amino acid entities in the composition have been carefully selected to combine to improve liver function in a subject (e.g., a subject having a liver disease or disorder) that requires the coordination of many biological, cellular, and molecular processes. This approach also applies to combinations of compositions (e.g., an Active Moiety) with therapeutic agents.

Described herein, in part, is a composition (e.g., an Active Moiety) comprising amino acid entities and methods of improving one, two, three, or four of liver function, hyperammonemia, muscle mass, or muscle function by administering an effective amount of the composition. The composition can be administered to treat or prevent a liver disease or disorder with one or both of hyperammonemia or muscle wasting in a subject in need thereof.

Sarcopenia is a significant complication of cirrhosis and is associated with overall mortality in patients with end-stage liver disease. Limited therapies aimed at ameliorating sarcopenia in cirrhosis are available despite the fact that decreased muscle mass represents a significant risk-factor for other complications of cirrhosis, such as ascites, infection, and hepatic encephalopathy. As the liver is an important tissue for amino acid homeostasis, amino acid profiles are perturbed in patients with cirrhosis, which further exacerbates muscle wasting and cirrhosis-associated complications. In addition to muscle mass, muscle quality has significant impact on cirrhosis and presence of fat infiltration in muscle can predict risk to hepatic encephalopathy. Moreover, presence of inflammation exacerbates defective muscle mass, quality, and metabolism and predicts disease progression.

The amino acid entities and relative amounts of the amino acid entities in the compositions disclosed herein have been optimized, e.g., to improve liver function, hyperammonemia, chronic inflammation, muscle function, muscle mass, and reduce complications associated with liver dysfunction (e.g., ascites, infection, or hepatic encephalopathy) in a subject that requires the coordination of many biological, cellular, and molecular processes. In some embodiments, the compositions disclosed herein improve ammonia detoxification within one or both of muscle or blood, while stimulating muscle anabolism, e.g., by improving the amino acid profile of a subject with a liver disease or disorder, such as cirrhosis.

Without being bound by any theory, it is understood that a composition of the invention can reprogram the disordered multifactorial cascade of ammonia-induced muscle wasting in liver diseases and disorders, such as cirrhosis, to improve one of more of: 1) a defective urea cycle (e.g., resulting in liver failure); 2) muscle wasting as a result of one or both of increased BCAA catabolism or deregulated mTORC1 signaling; 3) amino acid imbalance (e.g., a depletion of valine, isoleucine, and isoleucine with an enrichment of phenylalanine and tyrosine in plasma); 4) inflammation and resulting anabolic resistance and hypercatabolism; and 5) muscle metabolism. Similarly, administration of a composition of the invention can result in one, two, three or all of increase the Fischer’s ratio (e.g., the ratio of a level of BCAAs to a level of AAAs), increase the valine to phenylalanine ratio, improve body composition toward a leaner phenotype, and improve the utilization of amino acids towards muscle protein synthesis, e.g., to lower ammonia levels, or improve muscle quality in a subject.

In some embodiments Minimal Hepatic Encephalopathy tools (Stroop, NCT, PHES) may be used to assess expected improvements from the coordinated increases in muscle mass and quality, lowered inflammation, and improved metabolism by a composition of the invention.

In some embodiments, systemic inflammatory markers (e.g. LPS, TNF, CRP, interleukins, etc.) and myokines (e.g. IL-6, myostatin, FGFs, GDFs, etc.) may be improved by a composition of the invention.

In some embodiment muscle mass and quality are improved by a composition of the invention.

### Definitions

Terms used in the claims and specification are defined as set forth below unless otherwise specified.

It must be noted that, as used in the specification and the appended claims, the singular forms “a,” “an” and “the” include plural referents unless the context clearly dictates otherwise.

As used herein, the term “amino acid entity” refers to a levo (L)-amino acid in free form or salt form (or both), the L-amino acid residue in a peptide smaller than 20 amino acid residues (e.g., oligopeptide, e.g., a dipeptide or a tripeptide), a derivative of the amino acid, a precursor of the amino acid, or a metabolite of the amino acid (see, e.g., Table 1). An amino acid entity includes a derivative of the amino acid, a precursor of the amino acid, a metabolite of the amino acid, or a salt form of the amino acid that is capable of effecting biological functionality of the free L-amino acid. An amino acid entity does not include a naturally occurring polypeptide or protein of greater than 20 amino acid residues, either in whole or modified form, e.g., hydrolyzed form. Salts of amino acids include any ingestible salt. For pharmaceutical compositions, the salt form of an amino acid present in the composition (e.g., Active Moiety) should be a pharmaceutically acceptable salt. In a specific example, the salt form is the hydrochloride (HCl) salt form of the amino acid. In some embodiments, the derivative of an amino acid entity comprises an amino acid ester (e.g., an alkyl ester, e.g., an ethyl ester or a methyl ester of an amino acid entity) or a keto-acid.

TABLE 1

Amino acid entities include amino acids, precursors, metabolites, and derivatives of the compositions described herein

| | Exemplary Amino Acid | Precursors | Metabolites | Derivatives | Salts |
|---|---|---|---|---|---|
| Leucine | L-Leucine | Oxo-leucine | HMB (beta-hydroxy-beta-methybutyrate); Oxo-leucine; Isovaleryl-CoA | N-Acetyl-Leucine | |
| Isoleucine | L-Isoleucine | 2-Oxo-3-methyl-valerate; Threonine | 2-Oxo-3-methyl-valerate; Methylbutyrl-CoA | N-Acetyl-Isoleucine | |
| Arginine | L-Arginine | Argininosuccinat e; Citrulline; Aspartate; Glutamate | Ornithine; Citrulline; Agmatine; Creatine | N-Acetyl-Arginine | |
| Glutamine | L-Glutamine | Glutamate | Carbamoyl-P; Glutamate | N-AcetylGlutamine | |
| NAC | N-Acetylcyste ine | Serine; Acetylserine; Cystathionine | Glutathione; Cystathionine; Homocysteine; Methionine | L-Cysteine; Cystine; Cysteamine | |
| Serine | L-Serine | Phosphoserine, P-hydroxypyruvate, L-Glycine | Glycine, Tryptophan, Acetylserine, Cystathionine, Phosphatidylserine | | |
| Carnitine | L-Carnitine | 6-N-trimethyllysine; N6-Trimethyl-3-OH-lysine | | Acetyl-L-Carnitine (ALCAR); Proprionyl-L-Carnitine (PLCAR); L-Carnitine L-Tartrate | |
| Valine | L-Valine | 2-Oxo-valerate (alpha-ketoisovalerate (KIV) | Isobutryl-CoA | N-Acetyl-Valine | |
| Ornithine | L-Ornithine | L-Arginine, Glycine | Citrulline | | Ornithin e α-ketoglut arate, Ornithin e HCl |
| Aspartate | L-Aspartate | Fumarate | Adenylosuccinate | | |
| Histidine | L-Histidine | Histidinol; Histidinal; Ribose-5-phosphate | Carnosine; Histamine; Urocanate | N-Acetyl-Histidine | |
| Lysine | L-Lysine | Diaminopimelate; Aspartate | Trimethylhistidine amino acid entity; Carnitine; Saccharopine | N-Acetyl-Lysine | L-Lysine Acetate |
| Threonine | L-Threonine | Homoserine; O-PhosphoHomoser ine | Oxobutyrate | N-Acetyl-Threonine | |

For example, where XXX is leucine (L), then leucine amino acid entity refers to free L or L in salt form, a peptide (e.g., a dipeptide or a tripeptide) comprising a L residue, a L derivative, a L precursor, or a metabolite of L; where XXX is isoleucine(I), then isoleucine amino acid entity refers to free I or I in salt form, a peptide (e.g., a dipeptide or a tripeptide) comprising a I residue, a I derivative, a I precursor, or a metabolite of I; where XXX is valine (V), then valine amino acid entity refers to free V or V in salt form, a peptide (e.g., a dipeptide or a tripeptide) comprising a V residue, a V derivative, a V precursor, or a metabolite of V; where XXX is ornithine (Om), then ornithine amino acid entity refers to free Orn or Orn in salt form, a peptide (e.g., a dipeptide or a tripeptide) comprising a Orn residue, a Orn derivative, a Orn precursor, or a metabolite of Orn; where XXX is aspartate (D), then aspartate amino acid entity refers to free D or D in salt form, a peptide (e.g., a dipeptide or a tripeptide) comprising a D residue, a D derivative, a D precursor, or a metabolite of D; where XXX is histidine (H), then histidine amino acid entity refers to free H or H in salt form, a peptide(e.g., a dipeptide or a tripeptide) comprising a H residue, a H derivative, a H precursor, or a metabolite of H; where XXX is lysine (K), then lysine amino acid entity refers to free K or K in salt form, a peptide (e.g., a dipeptide or a tripeptide) comprising a K residue, a K derivative, a K precursor, or a metabolite of K; and where XXX is threonine (T), then threonine amino acid entity refers to free T or T in salt form, a peptide (e.g., a dipeptide or a tripeptide) comprising a T residue, a T derivative, a T precursor, or a metabolite of T.

“About” and “approximately” shall generally mean an acceptable degree of error for the quantity measured given the nature or precision of the measurements. Exemplary degrees of error are within 15 percent (%), typically, within 10%, and more typically, within 5% of a given value or range of values.

An “amino acid” refers to an organic compound having an amino group (—NH₂), a carboxylic acid group (—C(═O)OH), and a side chain bonded through a central carbon atom, and includes essential and non-essential amino acids and natural, non-proteinogenic, and unnatural amino acids. Unless otherwise indicated, amino acids referred to herein are L-isomers of amino acids.

As used herein, the term “Active Moiety” means a combination of two, three, four, five, six or more amino acid entities that, in aggregate, have the ability to have a physiological effect as described herein, e.g., improving liver function, hyperammonemia, muscle mass, or muscle function. For example, an Active Moiety can rebalance a metabolic dysfunction in a subject suffering from a disease or disorder. For example, an Active Moiety can treat a liver disease or disorder with one or both of hyperammonemia or muscle wasting. An Active Moiety of the invention can contain other biologically active ingredients. In some examples, the Active Moiety comprises a defined combination of two, three, or four or more amino acid entities, as set out in detail below. In other embodiments, the Active Moiety consists of a defined combination of amino acid entities, as set out in detail below.

The individual amino acid entities are present in the composition, e.g., Active Moiety, in various amounts or ratios, which can be presented as amount by weight (e.g., in grams), ratio by weight of amino acid entities to each other, amount by mole, amount by weight percent of the composition, amount by mole percent of the composition, caloric content, percent caloric contribution to the composition, etc. Generally, this disclosure will provide grams of amino acid entity in a dosage form, weight percent of an amino acid entity relative to the weight of the composition, e.g., the weight of all the amino acid entities and any other biologically active ingredient present in the composition, or in ratios. In some embodiments, the composition, e.g., Active Moiety, is provided as a pharmaceutically acceptable preparation (e.g., a pharmaceutical product).

By “a combination” or “in combination with,” it is not intended to imply that the composition comprising amino acid entities (e.g., an Active Moiety) or the one or more therapeutic agents must be administered at the same time and/or formulated for delivery together, although these methods of delivery are within the scope described herein. The one or more therapeutic agents in the combination can be administered concurrently with, prior to, or subsequent to, the composition comprising amino acid entities. The composition comprising amino acid entities and therapeutic agent can be administered in any order. In general, each agent will be administered at a dose and/or on a time schedule determined for that agent. It will further be appreciated that the one or more therapeutic agents and composition comprising amino acid entities utilized in this combination may be administered together in a single composition or administered separately in different compositions. In some embodiments, the dosages utilized in combination will be lower than those utilized individually.

The term “effective amount” as used herein means an amount of an active of the invention in a composition of the invention, particularly a pharmaceutical composition of the invention, which is sufficient to reduce a symptom and/or improve a condition to be treated (e.g., provide a desired clinical response). The effective amount of an active for use in a composition will vary with the particular condition being treated, the severity of the condition, the duration of treatment, the nature of concurrent therapy, the particular active being employed, the particular pharmaceutically-acceptable excipient(s) and/or carrier(s) utilized, and like factors with the knowledge and expertise of the attending physician. Administering an effective amount of the combination includes administering a unit dose that contains both the composition and the therapeutic agent as well as administering each of the composition and the therapeutic agent separately in any sequence, timing, and frequency; provided that the patient receives both actives at an amount and dose to provide the benefits of the invention, such as an improved therapeutic window, lower toxicity, reduction in adverse events. In some embodiments, a daily dose of an oral therapeutic agent can be given in coformulation with a daily dose of the composition of amino acid entities. In some embodiments, a daily dose of an oral therapeutic agent can be given in coformulation once per day with a composition of amino acid entities once per day and the composition of amino acid entities given alone once per day. In some embodiments, an infusion of a therapeutic agent (e.g., a biologic, e.g. peptide, polypeptide, or antibody) can be given once every week or every 2 weeks and the composition of amino acid entities given twice per day.

An “equivalent amount” of an amino acid entity is an amount that yields, physiologically, the same activity as that amount of the corresponding free amino acid for the amino acid entity.

A “pharmaceutical composition” described herein comprises at least one Active Moiety and a pharmaceutically acceptable carrier or excipient. In some embodiments, the pharmaceutical composition is used as a therapeutic. Other compositions, which need not meet pharmaceutical standards (GMP; pharmaceutical grade components) can be used as a nutraceutical, a medical food, or as a supplement, these are termed “consumer health compositions.”

The term “pharmaceutically acceptable” as used herein, refers to amino acids, materials, excipients, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In a specific embodiment, “pharmaceutically acceptable” means free of detectable endotoxin or endotoxin levels are below levels acceptable in pharmaceutical products.

In a specific embodiment, “pharmaceutically acceptable” means a standard used by the pharmaceutical industry or by agencies or entities (e.g., government or trade agencies or entities) regulating the pharmaceutical industry to ensure one or more product quality parameters are within acceptable ranges for a medicine, pharmaceutical composition, treatment, or other therapeutic. A product quality parameter can be any parameter regulated by the pharmaceutical industry or by agencies or entities, e.g., government or trade agencies or entities, including but not limited to composition; composition uniformity; dosage; dosage uniformity; presence, absence, and/or level of contaminants or impurities; and level of sterility (e.g., the presence, absence and/or level of microbes). Exemplary government regulatory agencies include: Federal Drug Administration (FDA), European Medicines Agency (EMA), SwissMedic, China Food and Drug Administration (CFDA), or Japanese Pharmaceuticals and Medical Devices Agency (PMDA).

The term “pharmaceutically acceptable excipient” refers to an ingredient in a pharmaceutical formulation, other than an active, which is physiologically compatible. A pharmaceutically acceptable excipient can include, but is not limited to, a buffer, a sweetener, a dispersion enhancer, a flavoring agent, a bitterness masking agent, a natural coloring, an artificial coloring, a stabilizer, a solvent, or a preservative. In a specific embodiment, a pharmaceutically acceptable excipient includes one or both of citric acid and lecithin.

The term “non-amino acid entity protein component,” as used herein, refers to a peptide (e.g., a polypeptide or an oligopeptide), a fragment thereof, or a degraded peptide. Exemplary non-amino acid entity protein components include, but are not limited to, one or more of whey protein, egg white protein, soy protein, casein, hemp protein, pea protein, brown rice protein, or a fragment or degraded peptide thereof.

The term “non-protein component,” as used herein, refers to any component of a composition other than a protein component. Exemplary non-protein components can include, but are not limited to, a saccharide (e.g., a monosaccharide (e.g., dextrose, glucose, or fructose), a disaccharide, an oligosaccharide, or a polysaccharide); a lipid (e.g., a sulfur-containing lipid (e.g., α-lipoic acid), a long chain triglyceride, an omega 3 fatty acid (e.g., EPA, DHA, STA, DPA, or ALA), an omega 6 fatty acid (GLA, DGLA, or LA), a medium chain triglyceride, or a medium chain fatty acid); a vitamin (e.g., vitamin A, vitamin E, vitamin C, vitamin D, vitamin B6, vitamin B12, biotin, or pantothenic acid); a mineral (zinc, selenium, iron, copper, folate, phosphorous, potassium, manganese, chromium, calcium, or magnesium); or a sterol (e.g., cholesterol).

A composition, formulation or product is “therapeutic” if it provides a desired clinical effect. A desired clinical effect can be shown by lessening the progression of a disease and/or alleviating one or more symptoms of the disease.

A “unit dose” or “unit dosage” comprises the drug product or drug products in the form in which they are marketed for use, with a specific mixture of the active and inactive components (excipients), in a particular configuration (e.g., a capsule shell, for example), and apportioned into a particular dose (e.g., in multiple stick packs).

As used herein, the terms “treat,” “treating,” or “treatment” of a liver disease or disorder refers to ameliorating a liver disease or disorder (e.g., slowing, arresting, or reducing the development of a liver disease or disorder or at least one of the clinical symptoms thereof); alleviating or ameliorating at least one physical parameter including those which may not be discernible by the patient; and/or preventing or delaying the onset or development or progression of a liver disease or disorder. The terms “treat,” “treating,” or “treatment” of liver disease or disorder or muscle wasting refers to ameliorating a liver disease or disorder with one or both of hyperammonemia or muscle wasting (e.g., slowing, arresting, or reducing the development of the liver disease or disorder with one or both of hyperammonemia or muscle wasting or at least one of the clinical symptoms thereof); alleviating or ameliorating at least one physical parameter including those which may not be discernible by the patient; and/or preventing or delaying the onset or development or progression of a liver disease or disorder with one or both of hyperammonemia or muscle wasting.

### Compositions Comprising Amino Acid Entities (e.g., Active Moieties) Useful to Treat NASH/NAFLD

The composition of the invention as described herein (e.g., an Active Moiety) comprises amino acid entities, e.g., at least two amino acid entities (e.g., a NAC entity and a CAR entity), e.g., at least five amino acid entities (e.g., six or seven amino acid entities). In some embodiments, at least one, two, three, four, five, or more (e.g., all) of the amino acid entities (e.g., (a)-(b) or (a)-(f)) in the composition is selected from Table 1.

In some embodiments, the composition comprises, consists essentially of, or consists of: (a) a leucine amino acid entity, (b) a arginine amino acid entity, (c) glutamine amino acid entity, (d) a N-acetylcysteine (NAC) entity, and (e) one or both of a serine amino acid entity or a carnitine entity. In some embodiments, the composition further comprises: (f) an isoleucine amino acid entity.

In certain embodiments, the leucine amino acid entity is chosen from L-leucine, β-hydroxy-β-methylbutyrate (HMB), oxo-leucine (α-ketoisocaproate (KIC)), isovaleryl-CoA, n-acetyl-leucine, or a combination thereof.

In certain embodiments, the isoleucine amino acid entity is chosen from L-isoleucine, 2-oxo-3-methyl-valerate (α-keto-beta-methylvaleric acid (KMV)), threonine, methylbutyrl-CoA, N-acetyl-isoleucine, or a combination thereof.

In certain embodiments, the arginine amino acid entity is chosen from L-arginine, creatine, ornithine, argininosuccinate, citrulline, aspartate, glutamate, agmatine, N-acetyl-arginine, or a combination thereof.

In certain embodiments, the glutamine amino acid entity is chosen from L-glutamine, glutamate, carbamoyl-P, glutamate, n-acetylglutamine, or a combination thereof.

In certain embodiments, the NAC entity is selected chosen from NAC, serine, acetylserine, cystathionine, cystathionine, homocysteine, glutathione, or a combination thereof.

In certain embodiments, the serine amino acid entity is chosen from L-serine, phosphoserine, p-hydroxypyruvate, glycine, acetylserine, cystathionine, phosphatidylserine, or a combination thereof. In some embodiments, the serine amino acid entity is chosen from L-serine or L-glycine. In one embodiment, the serine amino acid entity is L-serine. In another embodiment, the serine amino acid entity is L-glycine. In another embodiment, the serine amino acid entity is L-glycine and L-serine (e.g., L-glycine and L-serine at a wt. ratio of 1:1).

In certain embodiments, the carnitine entity is chosen from L-carnitine, 6-N-trimethyllysine, N6-trimethyl-3-OH-lysine, acetyl-L-carnitine, proprionyl-L-carnitine, L-carnitine L-tartrate, or a combination thereof.

In some embodiments, the composition comprises a combination of 19 or fewer, 18 or fewer, 17 or fewer, 16 or fewer, 15 or fewer, 14 or fewer, 13 or fewer, 12 or fewer, 11 or fewer, or 10 or fewer amino acid entities. In some embodiments, the composition comprises a combination of 5 to 19, 5 to 18, 5 to 17, 5 to 16, 5 to 15, 5 to 14, 5 to 13, 5 to 12, 5 to 11, or 5 to 10 different amino acid entities.

In some embodiments, one, two, three, four, five, or more (e.g., all) of (a)-(f) are in free amino acid form in the composition, e.g., at least: 35 wt. %, 40 wt. %, 42 wt. %, 45 wt. %, 50 wt. %, 75 wt. %, 80 wt. %, 90 wt. %, or more of the total wt. of amino acid entities or total components is one, two, three, four, five, or more (e.g., all) of (a)-(f) in free amino acid form in the composition (e.g., in dry form). In some embodiments, one, two, three, four, five or more (e.g., all) of (a)-(f) are in salt form in the composition, e.g., at least: 0.001 wt. %, 0.01 wt. %, 0.1 wt. %, 0.5 wt. %, 1 wt. %, 5 wt. %, or 10 wt. %, or more of the amino acid entities or total components is one, two, three, four, five, or more (e.g., all) of (a)-(f) in salt form in the composition. In some embodiments, one, two, three, four, five, or more (e.g., all) of (a)-(f) is provided as part of a dipeptide or tripeptide, e.g., in an amount of at least: 0.001 wt. %, 0.01 wt. %, 0.1 wt. %, 0.5 wt. %, 1 wt. %, 5 wt. %, or 10 wt. % or more of amino acid entities or total components of the composition.

In some embodiments, the composition (e.g., the Active Moiety) comprises, consists essentially of, or consists of: a) an leucine amino acid entity chosen from: i) L-leucine or a salt thereof, ii) a dipeptide or salt thereof, or tripeptide or salt thereof, comprising L-leucine, or iii) β-hydroxy-β-methylbutyrate (HMB) or a salt thereof; b) an arginine amino acid entity chosen from: i) L-arginine or a salt thereof, ii) a dipeptide or salt thereof, or tripeptide or salt thereof, comprising L-arginine, iii) ornithine or a salt thereof, iv) a dipeptide or salt thereof, or tripeptide or salt thereof, comprising ornithine, v) creatine or a salt thereof, or vi) a dipeptide or salt thereof, or tripeptide or salt thereof, comprising creatine; c) the glutamine amino acid entity is L-glutamine or a salt thereof or a dipeptide or salt thereof, or tripeptide or salt thereof, comprising L-glutamine; d) the NAC entity is NAC or a salt thereof or a dipeptide or salt thereof, comprising NAC; and e) one or both of: i) the serine amino acid entity is L-serine or a salt thereof or a dipeptide or salt thereof, or tripeptide or salt thereof, comprising L-serine; or b) the carnitine entity is L-carnitine or a salt thereof or a dipeptide or salt thereof, comprising L-carnitine.

In some embodiments, the composition (e.g., an Active Moiety) comprises, consists essentially of, or consists of: a) the leucine amino acid entity is L-leucine or a salt thereof; b) the arginine amino acid entity is L-arginine or a salt thereof; c) the glutamine amino acid entity is L-glutamine or a salt thereof; d) the NAC entity is NAC or a salt thereof; e) one or both of: i) the serine amino acid entity is L-serine or a salt thereof, or ii) the carnitine entity is L-carnitine or a salt thereof; and f) the isoleucine amino acid entity is L-isoleucine or a salt thereof.

In some embodiments, the composition is capable of one, two, three, four, five, six, seven, or more (e.g., all) of: decreasing or preventing liver fibrosis; decreasing or preventing liver injury; decreasing or preventing hepatocyte inflammation; improving, e.g., increasing, glucose tolerance; decreasing or preventing steatosis; decreasing or preventing hepatocyte ballooning; increasing liver fatty acid oxidation; or improving gut function. In some embodiments, the composition decreases or prevents one or both of liver fibrosis and liver injury. In some embodiments, the decreasing or preventing one or both of liver fibrosis or liver injury includes reducing a level of collagen, e.g., one or both of type I and III collagen. In some embodiments, the decreasing or preventing one or both of liver fibrosis or liver injury includes reducing a level or activity of one, two, three, four, five, six, seven, eight, nine, ten, or more (e.g., all) of Acta2; Col1a1; FGF-21; hydroxyproline; IL-1β; a MMP (e.g., MMP-13, MMP-2, MMP-9, MT1-MMP, MMP-3, or MMP-10); proC3; PIINP; αSMA; TGFβ; or TIMP (e.g., TIMP1 or TIMP2). In some embodiments, the composition decreases or prevents liver inflammation (e.g., hepatocyte inflammation). In some embodiments, the decreasing or preventing liver inflammation includes reducing a level or activity of one, two, three, four, five, six, seven, eight, nine, or more (e.g., all) of aspartate transaminase (AST); alanine transaminase (ALT); C-reactive protein; IL-1β; IL-2; MCP-1; MIP-1; NF-kB; or TNFα. In some embodiments, the decreasing or preventing liver inflammation includes increasing a level or activity of IL-10. In some embodiments, the composition decreases insulin resistance or increases glucose tolerance. In some embodiments, the decreasing insulin resistance or increasing glucose tolerance includes reducing a level or activity of one, two, three, four, five, or more (e.g., all) of ACOX1; caspase-cleaved keratin 18 fragments (e.g., M30 or M65); FGF-21; hydroxyproline content; IL-1β; or IL-2. In some embodiments, the decreasing insulin resistance or increasing glucose tolerance includes increasing a level or activity of adiponectin.

### i. Amounts

An exemplary composition can include 1 g of an leucine amino acid entity, 0.5 g of an isoleucine amino acid entity, 1.33 g of an arginine amino acid entity, 0.67 g of a glutamine amino acid entity, 0.43 g of a NAC entity, 0.30 g of a carnitine entity, and 2.5 of a serine amino acid entity for a total of 6.73 g +/- 20% (e.g., g/packet as shown in Table 2).

TABLE 2

Exemplary composition comprising amino acids (e.g., an Active Moiety)

| Amino Acid | Packet (g) | Dose (g) | Total Daily | Wt. Ratio | Wt % |
|---|---|---|---|---|---|
| L-leucine | 1.00 | 3.00 | 6.00 | 3.00 | 14.85 |
| L-isoleucine | 0.50 | 1.50 | 3.00 | 1.50 | 7.43 |
| L-arginine | 1.33 | 4.00 | 8.00 | 4.00 | 19.80 |
| L-glutamine | 0.67 | 2.00 | 4.00 | 2.00 | 9.90 |
| NAC | 0.43 | 1.30 | 2.60 | 1.30 | 6.44 |
| L-carnitine | 0.30 | 0.90 | 1.80 | 0.90 | 4.46 |
| L-serine | 2.50 | 7.50 | 15.00 | 7.50 | 37.13 |
| Total amino acids | 6.73 | 20.20 | 40.40 | 20.20 | 100.00 |

In some embodiments, the composition (e.g., the Active Moiety) includes 1 g +/- 20% of an leucine amino acid entity, 0.5 g +/- 20% of an isoleucine amino acid entity, 1.33 g +/- 20% of an arginine amino acid entity, 0.67 g +/- 20% of a glutamine amino acid entity, 0.43 g +/- 20% of a NAC entity, 0.30 g +/- 20% of a carnitine entity, and 2.5 g +/- 20% of a serine amino acid entity. In some embodiments, the composition (e.g., the Active Moiety) includes 1 g +/- 15% of an leucine amino acid entity, 0.5 g +/- 15% of an isoleucine amino acid entity, 1.33 g +/- 15% of an arginine amino acid entity, 0.67 g +/- 15% of a glutamine amino acid entity, 0.43 g +/- 15% of a NAC entity, 0.30 g +/- 15% of a carnitine entity, and 2.5 g +/- 15% of a serine amino acid entity. In some embodiments, the composition (e.g., the Active Moiety) includes 1 g +/- 10% of an leucine amino acid entity, 0.5 g +/- 10% of an isoleucine amino acid entity, 1.33 g +/- 10% of an arginine amino acid entity, 0.67 g +/- 10% of a glutamine amino acid entity, 0.43 g +/- 10% of a NAC entity, 0.30 g +/- 10% of a carnitine entity, and 2.5 g +/- 10% of a serine amino acid entity. In some embodiments, the composition (e.g., the Active Moiety) includes 1 g +/- 5% of an leucine amino acid entity, 0.5 g +/- 5% of an isoleucine amino acid entity, 1.33 g +/- 5% of an arginine amino acid entity, 0.67 g +/- 5% of a glutamine amino acid entity, 0.43 g +/- 5% of a NAC entity, 0.30 g +/- 5% of a carnitine entity, and 2.5 g +/- 5% of a serine amino acid entity.

### ii. Ratios

An exemplary composition can include a weight (wt.) ratio of the leucine amino acid entity, the arginine amino acid entity, the glutamine amino acid entity, and the NAC entity of 3 : 4 : 2 : 1.3. In some embodiments, the wt. ratio of the leucine amino acid entity, the isoleucine amino acid entity, the arginine amino acid entity, the glutamine amino acid entity, and the NAC entity is 3 +/-20% : 4 +/-20% : 2 +/-20% : 1.3 +/-20%. In some embodiments, the wt. ratio of the leucine amino acid entity, the isoleucine amino acid entity, the arginine amino acid entity, the glutamine amino acid entity, and the NAC entity is 3 +/-15% : 4 +/-15% : 2 +/-15% : 1.3 +/-15%. In some embodiments, the wt. ratio of the leucine amino acid entity, the isoleucine amino acid entity, the arginine amino acid entity, the glutamine amino acid entity, and the NAC entity is 3 +/-10% : 4 +/-10% : 2 +/-10% : 1.3 +/-10%. In some embodiments, the wt. ratio of the leucine amino acid entity, the isoleucine amino acid entity, the arginine amino acid entity, the glutamine amino acid entity, and the NAC entity is 3 +/-5% : 4 +/-5% : 2 +/-5% : 1.3 +/-5%.

An exemplary composition can include a wt. ratio of the leucine amino acid entity, the arginine amino acid entity, the glutamine amino acid entity, and the NAC entity of 3 : 1.5: 4 : 2 : 1.3. In some embodiments, the wt. ratio of the leucine amino acid entity, the isoleucine amino acid entity, the arginine amino acid entity, the glutamine amino acid entity, and the NAC entity is 3 +/-20% : 1.5 +/-20% : 4 +/-20% : 2 +/-20% : 1.3 +/-20%. In some embodiments, the wt. ratio of the leucine amino acid entity, the isoleucine amino acid entity, the arginine amino acid entity, the glutamine amino acid entity, and the NAC entity is 3 +/-15% : 1.5 +/-15% : 4 +/-15% : 2 +/-15% : 1.3 +/-15%. In some embodiments, the wt. ratio of the leucine amino acid entity, the isoleucine amino acid entity, the arginine amino acid entity, the glutamine amino acid entity, and the NAC entity is 3 +/-10% : 1.5 +/-10% : 4 +/-10% : 2 +/-10% : 1.3 +/-10%. In some embodiments, the wt. ratio of the leucine amino acid entity, the isoleucine amino acid entity, the arginine amino acid entity, the glutamine amino acid entity, and the NAC entity is 3 +/-5% : 1.5 +/-5% : 4 +/-5% : 2 +/-5% : 1.3 +/-5%.

An exemplary composition can include a wt. ratio of the leucine amino acid entity, the isoleucine amino acid entity, the arginine amino acid entity, the glutamine amino acid entity, the NAC entity, and the carnitine entity of 3 : 1.5 : 4 : 2 : 1.3 : 0.9. In some embodiments, the composition includes a wt. ratio of the leucine amino acid entity, the isoleucine amino acid entity, the arginine amino acid entity, the glutamine amino acid entity, the NAC entity, and the carnitine entity of 3 +/-20% : 1.5 +/-20% : 4 +/-20% : 2 +/-20% : 1.3 +/-20% : 0.9 +/-20%. In some embodiments, the composition includes a wt. ratio of the leucine amino acid entity, the isoleucine amino acid entity, the arginine amino acid entity, the glutamine amino acid entity, the NAC entity, and the carnitine entity of 3 +/-15% : 1.5 +/-15% : 4 +/-15% : 2 +/-15% : 1.3 +/-15% : 0.9 +/-15%. In some embodiments, the composition includes a wt. ratio of the leucine amino acid entity, the isoleucine amino acid entity, the arginine amino acid entity, the glutamine amino acid entity, the NAC entity, and the carnitine entity of 3 +/-10% : 1.5 +/-10% : 4 +/-10% : 2 +/-10% : 1.3 +/-10% : 0.9 +/-10%. In some embodiments, the composition includes a wt. ratio of the leucine amino acid entity, the isoleucine amino acid entity, the arginine amino acid entity, the glutamine amino acid entity, the NAC entity, and the carnitine entity of 3 +/-5% : 1.5 +/-5% : 4 +/-5% : 2 +/-5% : 1.3 +/-5% : 0.9 +/-5%.

An exemplary composition can include a wt. ratio of the leucine amino acid entity, the isoleucine amino acid entity, the arginine amino acid entity, the glutamine amino acid entity, the NAC entity, and the serine amino acid entity of 3 : 1.5 : 4 : 2 : 1.3 : 7.5. In some embodiments, the composition includes a wt. ratio of the leucine amino acid entity, the isoleucine amino acid entity, the arginine amino acid entity, the glutamine amino acid entity, the NAC entity, and the serine amino acid entity of 3 +/-20% : 1.5 +/-20% : 4 +/-20% : 2 +/-20% : 1.3 +/-20% : 7.5 +/-20%. In some embodiments, the composition includes a wt. ratio of the leucine amino acid entity, the isoleucine amino acid entity, the arginine amino acid entity, the glutamine amino acid entity, the NAC entity, and the serine amino acid entity of 3 +/-15% : 1.5 +/-15% : 4 +/-15% : 2 +/-15% : 1.3 +/-15% : 7.5 +/-15%. In some embodiments, the composition includes a wt. ratio of the leucine amino acid entity, the isoleucine amino acid entity, the arginine amino acid entity, the glutamine amino acid entity, the NAC entity, and the serine amino acid entity of 3 +/-10% : 1.5 +/-10% : 4 +/-10% : 2 +/-10% : 1.3 +/-10% : 7.5 +/-10%. In some embodiments, the composition includes a wt. ratio of the leucine amino acid entity, the isoleucine amino acid entity, the arginine amino acid entity, the glutamine amino acid entity, the NAC entity, and the serine amino acid entity of 3 +/-5% : 1.5 +/-5% : 4 +/-5% : 2 +/-5% : 1.3 +/-5% : 7.5 +/-5%.

An exemplary composition can include a wt. ratio of the leucine amino acid entity, the isoleucine amino acid entity, the arginine amino acid entity, the glutamine amino acid entity, the NAC entity, the carnitine entity, and the serine amino acid entity of 3 : 1.5 : 4 : 2 : 1.3 : 0.9 : 7.5. In some embodiments, the composition includes a wt. ratio of the leucine amino acid entity, the isoleucine amino acid entity, the arginine amino acid entity, the glutamine amino acid entity, the NAC entity, the carnitine entity, and the serine amino acid entity of 3 +/-20% : 1.5 +/-20% : 4 +/-20% : 2 +/-20% : 1.3 +/-20% : 0.9 +/-20% : 7.5 +/-20%. In some embodiments, the composition includes a wt. ratio of the leucine amino acid entity, the isoleucine amino acid entity, the arginine amino acid entity, the glutamine amino acid entity, the NAC entity, the carnitine entity, and the serine amino acid entity of 3 +/-15% : 1.5 +/-15% : 4 +/-15% : 2 +/-15% : 1.3 +/-15% : 0.9 +/-15% : 7.5 +/-15%. In some embodiments, the composition includes a wt. ratio of the leucine amino acid entity, the isoleucine amino acid entity, the arginine amino acid entity, the glutamine amino acid entity, the NAC entity, the carnitine entity, and the serine amino acid entity of 3 +/-10% : 1.5 +/-10% : 4 +/-10% : 2 +/-10% : 1.3 +/-10% : 0.9 +/-10% : 7.5 +/-10%. In some embodiments, the composition includes a wt. ratio of the leucine amino acid entity, the isoleucine amino acid entity, the arginine amino acid entity, the glutamine amino acid entity, the NAC entity, the carnitine entity, and the serine amino acid entity of 3 +/-5% : 1.5 +/-5% : 4 +/-5% : 2 +/-5% : 1.3 +/-5% : 0.9 +/-5% : 7.5 +/-5%.

### iii. Relationships of Amino Acid Entities

In some embodiments, the wt. % of the leucine amino acid entity in the composition (e.g., in dry form) is greater than the wt. % of the glutamine amino acid entity, e.g., the wt. % of the leucine amino acid entity in the composition (e.g., in dry form) is at least 15% greater than the wt. % of the glutamine amino acid entity entity, e.g., the wt. % of the leucine amino acid entity is at least 20%, 25%, or 30% greater than the wt. % of the glutamine amino acid entity.

In some embodiments, the wt. % of the leucine amino acid entity in the composition (e.g., in dry form) is greater than the wt. % of the isoleucine amino acid entity, e.g., the wt. % of the leucine amino acid entity in the composition (e.g., in dry form) is at least 25% greater than the wt. % of the isoleucine amino acid entity, e.g., the wt. % of the leucine amino acid entity is at least 30%, 40%, or 50% greater than the wt. % of the isoleucine amino acid entity.

In some embodiments, the wt. % of the leucine amino acid entity and the isoleucine amino acid entity in combination in the composition (e.g., in dry form) is greater than the wt. % of the glutamine amino acid entity in the composition (e.g., in dry form), e.g., the wt. % of the leucine amino acid entity and the isoleucine amino acid entity in combination in the composition (e.g., in dry form) is at least 25% greater than the wt. % of the glutamine amino acid entity, e.g., the wt. % of the leucine amino acid entity and the isoleucine amino acid entity in combination is at least 30%, 40%, or 50% greater than the wt. % of the glutamine amino acid entity.

In some embodiments, the isoleucine amino acid entity and the leucine amino acid entity in combination is at least: 15 wt. %, 20 wt. %, 25%, 30%, 35%, or 40% of the amino acid entities in the composition (e.g., in dry form), but not more than: 50 wt. %, 60 wt. %, 70 wt. %, or 80 wt. % of the amino acid entities in the composition (e.g., in dry form).

In some embodiments, the wt. % of the leucine amino acid entity and the isoleucine amino acid entity in combination in the composition (e.g., in dry form) is greater than the wt. % of the arginine amino acid entity in the composition (e.g., in dry form), e.g., the wt. % of the leucine amino acid entity and the isoleucine amino acid entity in combination in the composition (e.g., in dry form) is at least 5% greater than the wt. % of the arginine amino acid entity, e.g., the wt. % of the leucine amino acid entity and the isoleucine amino acid entity in combination is at least 6%, 7%, 8%, 9%, or 10% greater than the wt. % of the arginine amino acid entity.

In some embodiments, the wt. % of the arginine amino acid entity in the composition (e.g., in dry form) is greater than the wt. % of the glutamine amino acid entity, e.g., the wt. % of the arginine amino acid entity in the composition (e.g., in dry form) is at least 25% greater than the wt. % of the glutamine amino acid entity, e.g., the wt. % of the arginine amino acid entity is at least 30%, 35%, 40%, 45%, or 50% greater than the wt. % of the glutamine amino acid entity.

In some embodiments, the wt. % of the arginine amino acid entity in the composition (e.g., in dry form) is greater than the wt. % of the leucine amino acid entity, e.g., the wt. % of the arginine amino acid entity in the composition (e.g., in dry form) is at least 10% greater than the wt. % of the leucine amino acid entity, e.g., the wt. % of the arginine amino acid entity is at least 15%, 17%, 20%, 22%, or 25% greater than the wt. % of the leucine amino acid entity.

In some embodiments, the serine amino acid entity is present at a greater wt. % than one, two, or more (e.g., all) of any other amino acid entity component, non-amino acid entity protein component, or non-protein component in the composition (e.g., in dry form). In some embodiments, the wt. % of the serine amino acid entity is at least: 8 wt. %, 9 wt. %, 10 wt.%, 15 wt. %, 20 wt. %, 25 wt. %, 27 wt. %, 30 wt. %, 32 wt. %, or 35 wt. % of the total amino acid entity components or total components in the composition (e.g., in dry form), but not more than 70 wt. %, 75 wt. %, 80 wt. %, 85 wt. %, or 90 wt. % of the total amino acid entity components or total components in the composition (e.g., in dry form).

In some embodiments, the wt. % of the serine amino acid entity in the composition (e.g., in dry form) is greater than the wt. % of the leucine amino acid entity, e.g., the wt. % of the serine amino acid entity in the composition (e.g., in dry form) is at least 30% greater than the wt. % of the leucine amino acid entity, e.g., the wt. % of the serine amino acid entity is at least 45%, 47%, 50%, 53%, 55%, 57%, or 60% greater than the wt. % of the leucine amino acid entity.

In some embodiments, the wt. % of the serine amino acid entity in the composition (e.g., in dry form) is greater than the wt. % of the isoleucine amino acid entity, e.g., the wt. % of the serine amino acid entity in the composition (e.g., in dry form) is at least 50% greater than the wt. % of the isoleucine amino acid entity, e.g., the wt. % of the serine amino acid entity is at least 60%, 65%, 70%, 75%, or 80% greater than the wt. % of the isoleucine amino acid entity.

In some embodiments, the wt. % of the serine amino acid entity in the composition (e.g., in dry form) is greater than the wt. % of the arginine amino acid entity, e.g., the wt. % of the serine amino acid entity in the composition (e.g., in dry form) is at least 20% greater than the wt. % of the arginine amino acid entity, e.g., the wt. % of the serine amino acid entity is at least 35%, 37%, 40%, 42%, or 45% greater than the wt. % of the arginine amino acid entity.

In some embodiments, the wt. % of the serine amino acid entity in the composition (e.g., in dry form) is greater than the wt. % of the glutamine amino acid entity, e.g., the wt. % of the serine amino acid entity in the composition (e.g., in dry form) is at least 40% greater than the wt. % of the glutamine amino acid entity, e.g., the wt. % of the serine amino acid entity is at least 50%, 55%, 60%, 65%, or 70% greater than the wt. % of the glutamine amino acid entity.

In some embodiments, the wt. % of the serine amino acid entity in the composition (e.g., in dry form) is greater than the wt. % of the leucine amino acid entity and the isoleucine amino acid entity in combination, e.g., the wt. % of the serine amino acid entity in the composition (e.g., in dry form) is at least 20% greater than the wt. % of the leucine amino acid entity and the isoleucine amino acid entity in combination, e.g., the wt. % of the serine amino acid entity is at least 30%, 33%, 35%, 37%, or 40% greater than the wt. % of the leucine amino acid entity and the isoleucine amino acid entity in combination.

In some embodiments, the wt. % of the NAC entity is at least: 2 wt. %, 3 wt. % or 5 wt. % of the total amino acid entity components or total components in the composition (e.g., in dry form), but not more than 10 wt. % of the total amino acid entity components or total components in the composition (e.g., in dry form). In some embodiments, the wt. % of the NAC entity in the composition (e.g., in dry form) is greater than the wt. % of the carnitine entity, e.g., the wt. % of the NAC entity in the composition (e.g., in dry form) is at least 10% greater than the wt. % of the carnitine entity, e.g., the wt. % of NAC entity is at least 15%, 17%, 20%, 22%, 25%, 28%, or 30% greater than the wt. % of the carnitine entity.

In some embodiments, the wt. % of the carnitine entity is at least: 1 wt. %, 2 wt. %, 3 wt. %, or 4 wt. % of the total amino acid entity components or total components in the composition (e.g., in dry form), but not more than 15 wt. %, 20 wt. %, 22 wt. %, 25 wt. %, 27 wt. %, or 30 wt. % of the total amino acid entity components or total components in the composition (e.g., in dry form).

In some embodiments, the wt. % of the leucine amino acid entity, the arginine amino acid entity, the glutamine amino acid entity, and the NAC entity is at least: 20 wt. %, 25 wt. %, 30 wt. %, 35 wt. %, or 40 wt. % of the total amino acid entity components or total components in the composition (e.g., in dry form), but not more than 70 wt. %, 80 wt. %, 90 wt. %, or 95 wt. % of the total amino acid entity components or total components in the composition (e.g., in dry form).

In some embodiments, the wt. % of the leucine amino acid entity, the arginine amino acid entity, the glutamine amino acid entity, the NAC entity, and the serine entity is at least: 50 wt. %, 55 wt. %, 60 wt. %, 65 wt. %, 70 wt. %, or 75 wt. % of the total amino acid entity components or total components in the composition (e.g., in dry form), but not more than 80 wt. %, 90 wt. %, 92 wt. %, or 95 wt. % of the total amino acid entity components or total components in the composition (e.g., in dry form) in the composition (e.g., in dry form).

In some embodiments, the wt. % of the leucine amino acid entity, the arginine amino acid entity, the glutamine amino acid entity, the NAC entity, and the carnitine entity is at least: 25 wt. %, 35 wt. %, 40 wt. %, 45 wt. %, 50 wt. %, 55 wt. %, 60 wt. %, 70 wt. %, or 75 wt. % of the total amino acid entity components or total components in the composition (e.g., in dry form), but not more than 80 wt. %, 85 wt. %, 90 wt. %, or 95 wt. % of the total amino acid entity components or total components in the composition (e.g., in dry form).

In some embodiments, the wt. % of the leucine amino acid entity, the arginine amino acid entity, the glutamine amino acid entity, the NAC entity, and the serine amino acid entity is at least: 60 wt. %, 65 wt. %, 70 wt. %, 75 wt. %, or 80 wt. % of the total amino acid entity components or total components in the composition (e.g., in dry form), but not more than 90 wt. %, 92 wt. %, 95 wt. %, or 97 wt. % of the total amino acid entity components or total components in the composition (e.g., in dry form) in the composition (e.g., in dry form).

In some embodiments, the wt. % of the leucine amino acid entity, the arginine amino acid entity, the glutamine amino acid entity, the NAC entity, the serine amino acid entity, and the carnitine entity is at least: 70 wt. %, 75 wt. %, 80 wt. %, 85 wt. %, or 90 wt. % of the total amino acid entity components or total components in the composition (e.g., in dry form), but not more than 95 wt. %, 96 wt. %, 97 wt. %, or 98 wt. % of the total amino acid entity components or total components in the composition (e.g., in dry form) in the composition (e.g., in dry form).

In some embodiments, the wt. % of the glutamine amino acid entity is at least: 5 wt. %, 6 wt. %, 7 wt. %, 8 wt. %, or 9 wt. % of the total amino acid entity components or total components in the composition (e.g., in dry form), but not more than 20 wt. %, 22 wt. %, 25 wt. %, 30 wt. %, 35 wt. %, or 40 wt. % of the total amino acid entity components or total components in the composition (e.g., in dry form).

### iv. Amino Acid Molecules to Exclude or Limit from the Composition

In some embodiments, the composition does not comprise a peptide of more than 20 amino acid residues in length (e.g., protein supplement) chosen from or derived from one, two, three, four, five, or more (e.g., all) of egg white protein, soy protein, casein, hemp protein, pea protein, or brown rice protein, or if the peptide is present, the peptide is present at less than: 10 weight (wt.) %, 9 wt. %, 8 wt. %, 7 wt. %, 6 wt. %, 5 wt. %, 4 wt. %, 3 wt. %, 2 wt. %, 1 wt. %, 0.5 wt. %, 0.1 wt. %, 0.05 wt. %, 0.01 wt. %, 0.001 wt. %, or less, of the total wt. of non-amino acid entity protein components or total components in the composition (e.g., in dry form).

In some embodiments, dipeptides or salts thereof or tripeptides or salts thereof are present at less than: 10 wt. %, 9 wt. %, 8 wt. %, 7 wt. %, 6 wt. %, 5 wt. %, 4 wt. %, 3 wt. %, 2 wt. %, 1 wt. %, 0.5 wt. %, 0.1 wt. %, 0.05 wt. %, 0.01 wt. %, 0.001 wt. %, or less of the total wt. of amino acid entity components or total components in the composition (e.g., in dry form). In some embodiments, at least 50%, 60%, 70%, or more of the total grams of amino acid entity components in the composition (e.g., in dry form) are from one, two, three, four, five, or more (e.g., all) of (a)-(f). In some embodiments, at least: 50%, 60%, 70%, or more of the calories from amino acid entity components or total components in the composition (e.g., in dry form) are from one, two, three, four, five, or more (e.g., all) of (a)-(f).

In some embodiments, one, two, three, or more (e.g., all) of methionine, tryptophan, valine, or cysteine is absent from the composition, or if present, are present at less than: 10 wt. %, 9 wt. %, 8 wt. %, 7 wt. %, 6 wt. %, 5 wt. %, 4 wt. %, 3 wt. %, 2 wt. %, 1 wt. %, 0.5 wt. %, 0.1 wt. %, 0.05 wt. %, 0.01 wt. %, 0.001 wt. %, or less, e.g., of the total wt. of the composition (e.g., in dry form). In some embodiments, one, two, three, or more (e.g., all) of methionine, tryptophan, valine, or cysteine, if present, are present in free form. In some embodiments, one, two, three, or more (e.g., all) of methionine, tryptophan, valine, or cysteine, if present, are present in salt form. In some embodiments, valine is absent from the composition, or if present, is present at less than: 10 wt. %, 9 wt. %, 8 wt. %, 7 wt. %, 6 wt. %, 5 wt. %, 4 wt. %, 3 wt. %, 2 wt. %, 1 wt. %, 0.5 wt. %, 0.1 wt. %, 0.05 wt. %, 0.01 wt. %, 0.001 wt. %, or less, e.g., of the total wt. of the composition (e.g., in dry form). In some embodiments, methionine, tryptophan, valine, or cysteine, if present, may be present in an oligopeptide, polypeptide, or protein, with the proviso that the protein is not whey, casein, lactalbumin, or any other protein used as a nutritional supplement, medical food, or similar product, whether present as intact protein or protein hydrolysate.

In some embodiments, phenylalanine is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form). In some embodiments, tyrosine is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form). In some embodiments, proline is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, a carbohydrate (e.g., one, two, three, four, five, six, seven, eight, nine, 10, 11, 12, 13, 14, 15, 16, 17, or 18 of dextrose, maltodextrose, sucrose, dextrin, fructose, galactose, glucose, glycogen, high fructose corn syrup, honey, inositol, invert sugar, lactose, levulose, maltose, molasses, sugarcane, or xylose) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, a vitamin (e.g., one, two, three, four, five, six, or seven of vitamin B1, vitamin B2, vitamin B3, vitamin B6, vitamin B 12, vitamin C, or vitamin D) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, one or both of nitrate or nitrite are absent from the composition, or if present, are present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, 4-hydroxyisoleucine is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, a probiotic (e.g., a Bacillus probiotic) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, phenylacetate is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, gelatin (e.g., a gelatin capsule) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

### Exemplary Therapeutic Agents

In some embodiments, the combination comprises a therapeutic agent chosen from one or both of the following categories: (i) a hepatic fat accumulation inhibitor; or (ii) one or both of an oxidative stress or hepatic inflammation inhibitor.

In certain embodiments, the hepatic fat accumulation inhibitor is chosen from an acetyl-CoA carboxylase inhibitor, an FXR agonist (e.g., a non-bile acid FXR agonist), or a combination thereof. In certain embodiments, the one or both of an oxidative stress or hepatic inflammation inhibitor is chosen from an ASK1 inhibitor.

### Exemplary ASK1 Inhibitors

In an embodiment, the combination includes an apoptosis signal-regulating kinase (ASK1) inhibitor. In some embodiments, use of the combination results in one, two, or more (e.g., all) of a functional change in cells (e.g., hepatocytes), reduction in inflammation, or reduction in fibrosis. In some embodiments, use of the combination results in an improvement in one, two, or more (e.g., all) of increased lipid metabolism, decreased inflammation, or decreased fibrosis. In some embodiments, use of the combination results in one or both of prevention or decreased hepatocyte apoptosis. In some embodiments, use of the combination results in one, two, three, four, five, or more (e.g., all) of a functional change in cells (e.g., hepatocytes), reduction in inflammation, reduction in fibrosis, increased lipid metabolism, decreased inflammation, or prevention or decrease of hepatocyte apoptosis, where the effect of the combination is greater than the therapeutic agent alone or a comparable effect is achieved with a lower dose of therapeutic agent in combination. In some embodiments, the combination is used to improve liver function or treat a liver disease or disorder described herein, e.g., a fatty liver disease or disorder (e.g., NAFLD (e.g., NASH or NAFL) or AFLD (e.g., ASH)).

In some embodiments, the ASK1 inhibitor is a compound disclosed in U.S. Pat. No. 8,742,126 (e.g., a compound of Formula (I) or a salt or derivative thereof), incorporated herein by reference in its entirety. In some embodiments, the ASK1 inhibitor is a compound disclosed in U.S. Pat. No. 8,552,196 (e.g., a compound of Formula (I) or a salt or derivative thereof), incorporated herein by reference in its entirety. In some embodiments, the ASK1 inhibitor is selonsertib (GS-4997 or SYN-1231; CAS 1448428-04-3).

In an embodiment, the ASK1 inhibitor is a compound of the following structure:

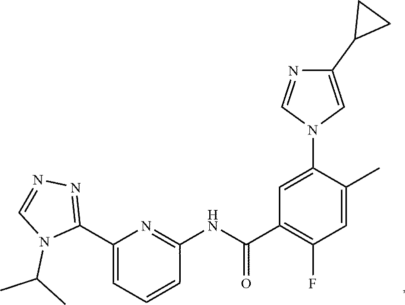

or a salt or derivative thereof.

In an embodiment, the ASK1 inhibitor is 5-(4-cyclopropyl-1H-imidazol-1-yl)-2-fluoro-N-(6-(4-isopropyl-4H-1,2,4-triazol-3-yl)pyridin-2-yl)-4-methylbenzamide, or a salt or derivative thereof.

Other exemplary ASK1 inhibitors are disclosed, e.g., in U.S. Pat. Publication Nos. 2017/217933, 2017/209423, 2017/273952, 2016/166556, or 2015/342943, or in U.S. Pat. Nos. 9,771,328, 9,750,730, 9,586,933, 9,643,956, 9,254,284, or 9,333,197, each of which is incorporated herein by reference in its entirety.

The ASK1 inhibitor (e.g., selonsertib) may be administered to a subject at a dose of 0.01 mg +/- 15% to 500 mg +/- 15% (e.g., referred herein as a monotherapy dose). In some embodiments, the ASK1 inhibitor (e.g., selonsertib) is administered to a subject at a dose of 0.1 mg +/- 15% to 250 mg +/- 15%, e.g., 0.5 mg +/- 15% to 100 mg +/- 15%, 1.5 mg +/- 15% to 50 mg +/- 15%, or 2 mg +/- 15% to 25 mg +/- 15%. In some embodiments, the ASK1 inhibitor (e.g., selonsertib) is administered to a subject at a dose of 2 mg +/- 15%, 6 mg +/- 15%, or 18 mg +/- 15%, e.g., once daily, twice daily, three times daily, four times daily, or five times daily (e.g., once daily). In an embodiment, the selonsertib is administered to a subject at a dose of 2 mg +/-15%, 6 mg +/- 15%, or 18 mg +/- 15%, e.g., once daily.

In certain embodiments, the administered amount or dosage of the ASK1 inhibitor (e.g., selonsertib) is lower (e.g., at least 20%, at least 30%, at least 40%, or at least 50%) than the amount or dosage when used individually, e.g., as a monotherapy (e.g., a monotherapy dose as described herein). In other embodiments, the amount or dosage of the ASK1 inhibitor (e.g., selonsertib) that results in a desired effect (e.g., treatment of a liver disease or disorder) is lower (e.g., at least 20%, at least 30%, at least 40%, or at least 50% lower).

### Exemplary Acetyl-CoA Carboxylase Inhibitors

In an embodiment, the combination includes an acetyl-CoA carboxylase inhibitor. In some embodiments, use of the combination results in one or both of an inhibition of hepatic fatty acid synthesis or reduction in steatosis. In some embodiments, use of the combination results in an improvement in one, two, or more (e.g., all) of increased lipid metabolism, decreased inflammation, or decreased fibrosis. In some embodiments, use of the combination results in one, two, three, four, or more (e.g., all) of inhibition of hepatic fatty acid synthesis, reduction in steatosis, increased lipid metabolism, decreased inflammation, or decreased fibrosis, where the effect of the combination is greater than the therapeutic agent alone or a comparable effect is achieved with a lower dose of therapeutic agent in combination. In some embodiments, the combination is used to improve liver function or treat a liver disease or disorder described herein, e.g., a fatty liver disease or disorder (e.g., NAFLD (e.g., NASH or NAFL) or AFLD (e.g., ASH)).

In some embodiments, the acetyl-CoA carboxylase inhibitor is a compound depicted in U.S. Pat. Publication No. 2018/0021341 (e.g., a compound of Formula (I) or a compound depicted in claim 6 or a salt or derivative thereof), incorporated herein by reference in its entirety. In some embodiments, the acetyl-CoA carboxylase inhibitor is GS 0976 (ND 630, firsocostat, or NDI 010976; CAS 1434635-54-7). In some embodiments, the acetyl-CoA carboxylase inhibitor is PF-05221304.

In an embodiment, the acetyl-CoA carboxylase inhibitor is a compound of the following structure:

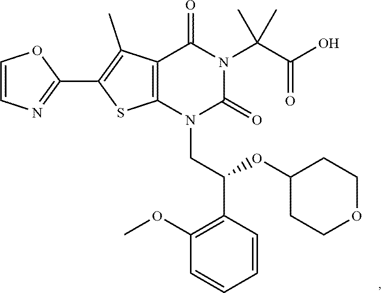

or a salt or derivative thereof.

In an embodiment, the acetyl-CoA carboxylase inhibitor is (R)-2-(1-(2-(2-methoxyphenyl)-2-((tetrahydro-2H-pyran-4-yl)oxy)ethyl)-5-methyl-6-(oxazol-2-yl)-2,4-dioxo-1,4-dihydrothieno[2,3-d]pyrimidin-3(2H)-yl)-2-methylpropanoic acid, or a salt or derivative thereof.

The acetyl-CoA carboxylase inhibitor (e.g., GS 0976) may be administered to a subject at a dose of 0.1 mg +/- 15% to 3000 mg +/- 15% (e.g., referred herein as a monotherapy dose). In some embodiments, the acetyl-CoA carboxylase inhibitor (e.g., GS 0976) is administered to a subject at a dose of 1 mg +/- 15% to 1000 mg +/- 15%, e.g., 2 mg +/- 15% to 500 mg +/- 15% or 5 mg +/- 15% to 100 mg +/- 15%. In some embodiments, the acetyl-CoA carboxylase inhibitor (e.g., GS 0976) is administered to a subject at a dose of 5 mg +/- 15%, or 20 mg +/- 15%, e.g., once daily, twice daily, three times daily, four times daily, or five times daily (e.g., once daily). In an embodiment, the GS 0976 is administered to a subject at a dose of 5 mg +/- 15%, or 20 mg +/- 15%, e.g., once daily.

The acetyl-CoA carboxylase inhibitor (e.g., PF-05221304) may be administered to a subject at a dose of 0.001 mg/kg +/- 15% to 10 mg/kg +/- 15% (e.g., referred herein as a monotherapy dose). In some embodiments, the acetyl-CoA carboxylase inhibitor (e.g., PF-05221304) is administered to a subject at a dose of 0.01 mg/kg +/- 15% to 5 mg/kg +/- 15%, e.g., 0.1 mg/kg +/- 15% to 4 mg/kg +/- 15% or 1 mg/kg +/- 15% to 3 mg/kg +/- 15%. In some embodiments, the acetyl-CoA carboxylase inhibitor (e.g., PF-05221304) is administered to a subject at a dose of 1 mg +/- 15%, 3 mg +/- 15%, 10 mg +/- 15%, 30 mg +/- 15%, 40 mg +/-15%, or 100 mg +/- 15%, e.g., once daily, twice daily, three times daily, four times daily, or five times daily (e.g., once daily or twice daily). In an embodiment, the PF-05221304 is administered to a subject at a dose of 1 mg +/- 15%, 3 mg +/- 15%, 10 mg +/- 15%, 30 mg +/- 15%, 40 mg +/-15%, or 100 mg +/- 15%, e.g., once daily or twice daily.

In certain embodiments, the administered amount or dosage of the acetyl-CoA carboxylase inhibitor (e.g., GS 0976, or PF-05221304) is lower (e.g., at least 20%, at least 30%, at least 40%, or at least 50%) than the amount or dosage when used individually, e.g., as a monotherapy (e.g., a monotherapy dose as described herein). In other embodiments, the amount or dosage of the acetyl-CoA carboxylase inhibitor (e.g., GS 0976, or PF-05221304) that results in a desired effect (e.g., treatment of a liver disease or disorder) is lower (e.g., at least 20%, at least 30%, at least 40%, or at least 50% lower).

### Exemplary FXR Agonists

In an embodiment, the combination includes an farnesoid receptor (FXR) agonist. In some embodiments, use of the combination results in one, two, or more (e.g., all) of an alteration in lipid metabolism, an alteration in bile acid metabolism, or reduction in fibrosis. In some embodiments, use of the combination results in one or both of decreased inflammation or decreased fibrosis. In some embodiments, use of the combination results in a complimentary effect on one or both of lipid metabolism or amino acid metabolism. In some embodiments, use of the combination results in one, two, three, four, five, or more (e.g., all) one or more (e.g., all) of alteration in lipid metabolism, an alteration in bile acid metabolism, reduction in fibrosis, decreased inflammation, decreased fibrosis, or a complimentary effect on one or both of lipid metabolism or amino acid metabolism, where the effect of the combination is greater than the therapeutic agent alone or a comparable effect is achieved with a lower dose of therapeutic agent in combination. In some embodiments, the combination is used to improve liver function or treat a liver disease or disorder described herein, e.g., a fatty liver disease or disorder (e.g., NAFLD (e.g., NASH or NAFL) or AFLD (e.g., ASH)).

In some embodiments, the FXR agonist is a compound depicted in U.S. Pat. No. 9,139,539 (e.g., a compound of Formula (1) or a compound depicted in claim 7 or a salt or derivative thereof), incorporated herein by reference in its entirety. In some embodiments, the FXR agonist is a compound depicted in U.S. Pat. No. 9,150,568 (e.g., a compound of Formula I or a compound depicted in claim 24 or a salt or derivative thereof), incorporated herein by reference in its entirety. In some embodiments, the FXR agonist is GS-9674 (cilofexor or PX0104; CAS 1418274-28-8). In some embodiments, the FXR agonist is LJN-452 (tropifexor or LMB763; CAS 1383816-29-2).

In an embodiment, the FXR agonist is a compound of the following structure:

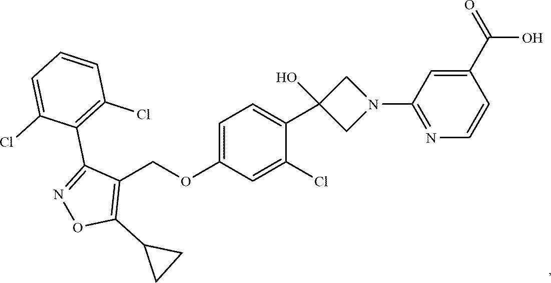

or a salt or derivative thereof.

In an embodiment, the FXR agonist is 2-(3-(2-chloro-4-((5-cyclopropyl-3-(2,6-dichlorophenyl)isoxazol-4-yl)methoxy)phenyl)-3-hydroxyazetidin-1-yl)isonicotinic acid, or a salt or derivative thereof.

In an embodiment, the FXR agonist is a compound of the following structure:

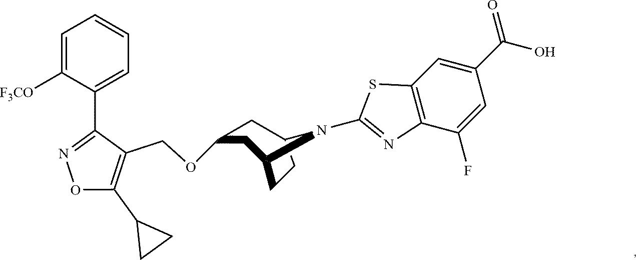

or a salt or derivative thereof.

In an embodiment, the FXR agonist is 2-[(3-endo)-3-[[5-cyclopropyl-3-[2-(trifluoromethoxy)phenyl]-4-isoxazolyl]methoxy]-8-azabicyclo[3.2.1]oct-8-yl]-4-fluoro-6-benzothiazolecarboxylic acid, or a salt or derivative thereof.

In an embodiment, the FXR agonist is a compound of the following structure:

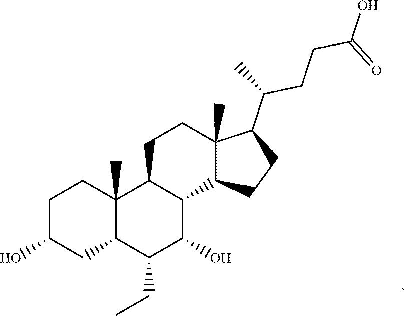

or a salt or a derivative thereof.

In an embodiment, the FXR agonist is obeticholic acid.

Other exemplary FXR agonists are disclosed, e.g., in U.S. Pat. Publication No. 2013/0261108, or U.S. Pat. No. 9,932,332, each of which is incorporated herein by reference in its entirety.

The FXR agonist (e.g., GS-9674) may be administered to a subject at a dose of 1 mg +/-15% to 1000 mg +/- 15% (e.g., referred herein as a monotherapy dose). In some embodiments, the FXR agonist (e.g., GS-9674) is administered to a subject at a dose of 2 mg +/- 15% to 500 mg +/- 15%, e.g., 10 mg +/- 15% to 300 mg +/- 15% or 20 mg +/- 15% to 150 mg +/- 15%. In some embodiments, the FXR agonist (e.g., GS-9674) is administered to a subject at a dose of 30 mg +/- 15%, or 100 mg +/- 15%, e.g., once daily, twice daily, three times daily, four times daily, or five times daily (e.g., once daily). In an embodiment, the GS-9674 is administered to a subject at a dose of 30 mg +/- 15%, or 100 mg +/- 15%, e.g., once daily.

The FXR agonist (e.g., LJN-452) may be administered to a subject at a dose of 1 mg +/-15% to 1000 mg +/- 15% (e.g., referred herein as a monotherapy dose). In some embodiments, the FXR agonist (e.g., LJN-452) is administered to a subject at a dose of 2 mg +/- 15% to 500 mg +/- 15%, e.g., 5 mg +/- 15% to 250 mg +/- 15% or 10 mg +/- 15% to 150 mg +/- 15%.

The FXR agonist (e.g., obeticholic acid) may be administered to a subject at a dose of 0.01 mg/kg +/- 15% to 100 mg/kg +/- 15% (e.g., referred herein as a monotherapy dose). In some embodiments, the FXR agonist (e.g., obeticholic acid) is administered to a subject at a dose of 0.1 mg/kg +/- 15% to 10 mg/kg +/- 15%, e.g., 0.15 mg/kg +/- 15% to 5 mg/kg +/- 15% or 0.2 mg/kg +/- 15% to 2 mg/kg +/- 15%. In an embodiment, the FXR agonist (e.g., obeticholic acid) is administered to a subject at a dose of 5 mg +/- 15%, 10 mg +/- 15%, 20 mg +/- 15%, or 25 mg +/- 15%, e.g., once daily, twice daily, three times daily, four times daily, or five times daily (e.g., once daily). In an embodiment, the obeticholic acid is administered to a subject at a dose of 5 mg +/- 15%, 10 mg +/- 15%, 20 mg +/- 15%, or 25 mg +/- 15%, e.g., once daily.

In certain embodiments, the administered amount or dosage of the FXR agonist (e.g., GS-9674, LJN-452, or obeticholic acid) is lower (e.g., at least 20%, at least 30%, at least 40%, or at least 50%) than the amount or dosage when used individually, e.g., as a monotherapy (e.g., a monotherapy dose as described herein). In other embodiments, the amount or dosage of the FXR agonist (e.g., GS-9674, LJN-452, or obeticholic acid) that results in a desired effect (e.g., treatment of a liver disease or disorder) is lower (e.g., at least 20%, at least 30%, at least 40%, or at least 50% lower).

### Compositions Comprising Amino Acid Entities (e.g., Active Moieties) Useful for Treating Cirrhosis and/or Hepatic Encephalopathy

The composition of the invention as described herein (e.g., an Active Moiety) comprises amino acid entities, e.g., the amino acid entities shown in Table 1.

In certain embodiments, the leucine amino acid entity is chosen from Table 1, e.g., the leucine amino acid entity is chosen from L-leucine, β-hydroxy-β-methylbutyrate (HMB), oxo-leucine (alpha-ketoisocaproate (KIC)), isovaleryl-CoA, n-acetylleucine, or a combination thereof. In certain embodiments, the leucine amino acid entity is chosen from L-leucine, oxo-leucine (KIC), isovaleryl-CoA, n-acetyl-Leucine, or a combination thereof.

In certain embodiments, the isoleucine amino acid entity is chosen from Table 1, e.g., the isoleucine amino acid entity is chosen from L-isoleucine, 2-oxo-3-methyl-valerate (alpha-keto-beta-methylvaleric acid (KMV)), threonine, methylbutyryl-CoA, D-isoleucine, N-acetyl-isoleucine, or a combination thereof.

In certain embodiments, the valine amino acid entity is chosen from Table 1, e.g., the valine amino acid entity is chosen from L-valine, 2-oxo-valerate (alpha-ketoisovalerate (KIV)), isobutyryl-CoA, N-acetyl-valine, or a combination thereof.

In certain embodiments, the ornithine amino acid entity is chosen from Table 1, e.g., the ornithine amino acid entity is chosen from L-ornithine, ornithine α-ketoglutarate, ornithine HCl, L-arginine, glycine, citrulline, or a combination thereof. In certain embodiments, the ornithine amino acid entity is chosen from L-ornithine, ornithine α-ketoglutarate, ornithine HCl, citrulline, or a combination thereof. In certain embodiments, the ornithine amino acid entity is chosen from L-ornithine, ornithine HCl, citrulline, or a combination thereof.

In certain embodiments, the aspartate amino acid entity is chosen from Table 1, e.g., the aspartate amino acid entity is chosen from L-aspartate, fumarate, adenylosuccinate, or a combination thereof.

In certain embodiments, the histidine amino acid entity is chosen from Table 1, e.g., the histidine amino acid entity is chosen from L-histidine, histidinol, histidinal, ribose-5-phosphate, carnosine, histamine, urocanate, and N-acetyl-histidine, or a combination thereof.

In certain embodiments, the lysine amino acid entity is chosen from Table 1, e.g., the lysine amino acid entity is chosen from L-lysine, diaminopimelate, aspartate, trimethylhistidine amino acid entity, carnitine, saccharopine, N-acetyl-Lysine, or a combination thereof.

In certain embodiments, the threonine amino acid entity is chosen from Table 1, e.g., the threonine amino acid entity is chosen from L-threonine, homoserine, O-phosphohomoserine, oxobutyrate, N-acetyl-threonine, or a combination thereof.

In some embodiments, one, two, or three of (a) a leucine amino acid entity, an isoleucine amino acid entity, or a valine amino acid entity is in free amino acid form. In some embodiments, one, two, or three of (a) a leucine amino acid entity, an isoleucine amino acid entity, a valine amino acid entity is in salt amino acid form.

In some embodiments, one or both of (b) an ornithine amino acid entity or an aspartate amino acid entity is in free amino acid form. In some embodiments, one or both of (b) ornithine amino acid entity or an aspartate amino acid entity is in salt amino acid form (e.g., L-ornithine or a salt thereof and L-aspartate or a salt thereof are present in combination as a salt (LOLA)).

In some embodiments, one, two, or three of (c) a histidine amino acid entity, a lysine amino acid entity, or a threonine amino acid entity is in free amino acid form. In some embodiments, one, two, or three of (c) a histidine amino acid entity, a lysine amino acid entity, or a threonine amino acid entity is in salt amino acid form (e.g., L-lysine or a salt thereof is present as L-lysine acetate).

In some embodiments, at least: 35 wt. %, 40 wt. %, 42 wt. %, 45 wt. %, 50 wt. %, 55 wt. %, 60 wt. %, 70 wt. %, 80 wt. %, or more, of the total wt. of the composition (in dry form) is three, four, five, six, seven, eight, nine, ten, or more (e.g., all) amino acid entities in (a)-(d) in free amino acid form. In some embodiments, at least: 15 wt. %, 20 wt. %, 25 wt. %, 35 wt. %, 40 wt. %, or more, of the total wt. of the composition (in dry form) is three, four, five, six, seven, eight, nine, ten, or more (e.g., all) amino acid entities in (a)-(d) in salt form.

In some embodiments, three, four, five, six, seven, eight, nine, ten, or more (e.g., all) amino acid entities in (a)-(d) is provided as part of a dipeptide or tripeptide, e.g., in an amount of at least: 0.01 wt. %, 0.1 wt. %, 0.5 wt. %, 1 wt. %, 5 wt. %, or 10 wt. %, or more of amino acid entities or total components of the composition.

In some embodiments, the composition further comprises L-alanine, L-arginine, L-tryptophan, carnitine, sodium acetate, or a combination thereof. In some embodiments, the composition further comprises a mineral, e.g., zinc. In some embodiments, the composition further comprises a vitamin, e.g., one, two, or three of vitamin A, vitamin D, vitamin E, or a combination thereof. In some embodiments, the composition further comprises an ammonia scavenger, e.g., phenyl acetate, acetyl-L-carnitine, citrulline, sodium benzoate, sodium phenylbutyrate, or a combination thereof.

In some embodiments, the composition can include sulfur AAs (SAAs), such as N-acetylcysteine (NAC). In an embodiment, the SAA (e.g., NAC) has anti-oxidant activity. In an embodiment, the SAA (e.g., NAC) results in decreased reactive oxygen species (ROS) or increased glutathione (GSH) in a subject administered the composition described herein.

In some embodiments, the composition comprises, consists of, or consists essentially of: N-acetylcysteine (NAC) or a salt thereof, or a dipeptide or a salt thereof, comprising NAC; and L-carnitine or a salt thereof, or a dipeptide or a salt thereof, comprising L-carnitine; or acetyl-L-carnitine (ALCAR) or a salt thereof or a dipeptide or salt thereof, or a tripeptide or salt thereof, comprising ALCAR. In some embodiments the total weight (wt.) % of N-acetylcysteine (NAC) or a salt thereof, or a dipeptide or a salt thereof, comprising NAC; and L-carnitine or a salt thereof, or a dipeptide or a salt thereof, comprising L-carnitine; or acetyl-L-carnitine (ALCAR) or a salt thereof or a dipeptide or salt thereof, or a tripeptide or salt thereof, comprising ALCAR is greater than 50% of the total wt. of amino acid entities. In some embodiments, the wt. % of L-carnitine or a salt thereof, or a dipeptide or a salt thereof, comprising L-carnitine; or acetyl-L-carnitine (ALCAR) or a salt thereof or a dipeptide or salt thereof, or a tripeptide or salt thereof, comprising ALCAR is at least 2% and up to 10% of the total wt. of amino acid entities in the composition.

In some embodiments, the composition comprises, consists of, or consists essentially of: a leucine amino acid entity, an isoleucine amino acid entity, valine amino acid entity , an ornithine amino acid entity, an aspartate amino acid entity, a histidine amino acid entity, a threonine amino acid entity , a lysine amino acid entity, and one or two of: i) N-acetylcysteine (NAC) or a salt thereof, or a dipeptide or a salt thereof, comprising NAC; or ii) L-carnitine or a salt thereof, or a dipeptide or a salt thereof, comprising L-carnitine; or acetyl-L-carnitine (ALCAR) or a salt thereof or a dipeptide or salt thereof, or a tripeptide or salt thereof, comprising ALCAR.

In some embodiments, the composition (e.g., the Active Moiety) comprises, consists of, or consists essentially of: a) a leucine amino acid entity; b) an ornithine amino acid entity; c) an essential amino acid (EAA)-entity chosen from a histidine amino acid entity, a lysine amino acid entity, or a threonine amino acid entity or a combination of two or three EAA entities; and d) one or two of: i) N-acetylcysteine (NAC) or a salt thereof, or a dipeptide or a salt thereof, comprising NAC; or ii) L-carnitine or a salt thereof, or a dipeptide or a salt thereof, comprising L-carnitine; or acetyl-L-carnitine (ALCAR) or a salt thereof or a dipeptide or salt thereof, or a tripeptide or salt thereof, comprising ALCAR; wherein at least one amino acid entity (e.g., two, three, four, or five amino acid entities) of (a)-(c) is not provided as a peptide of more than 20 amino acid residues in length.

In some embodiments, the composition (e.g., the Active Moiety) comprises, consists of, or consists essentially of: a) a leucine amino acid entity and a valine amino acid entity; b) an ornithine amino acid entity; c) an essential amino acid (EAA)-entity chosen from a histidine amino acid entity, a lysine amino acid entity, or a threonine amino acid entity or a combination of two or three EAA entities; and d) one or two of: i) N-acetylcysteine (NAC) or a salt thereof, or a dipeptide or a salt thereof, comprising NAC; or ii) L-carnitine or a salt thereof, or a dipeptide or a salt thereof, comprising L-carnitine; or acetyl-L-carnitine (ALCAR) or a salt thereof or a dipeptide or salt thereof, or a tripeptide or salt thereof, comprising ALCAR; wherein at least one amino acid entity (e.g., two, three, four, or five amino acid entities) of (a)-(c) is not provided as a peptide of more than 20 amino acid residues in length. In some embodiments, the composition further comprises an isoleucine amino acid entity. In some embodiments, the composition further comprises an aspartate amino acid entity.

In some embodiments, the composition (e.g., the Active Moiety) comprises, consists of, or consists essentially of: a) a leucine amino acid entity, an isoleucine amino acid entity, and a valine amino acid entity; b) an ornithine amino acid entity; c) an essential amino acid (EAA)-entity chosen from a histidine amino acid entity, a lysine amino acid entity, or a threonine amino acid entity or a combination of two or three EAA entities; and d) one or two of: i) N-acetylcysteine (NAC) or a salt thereof, or a dipeptide or a salt thereof, comprising NAC; or ii) L-carnitine or a salt thereof, or a dipeptide or a salt thereof, comprising L-carnitine; or acetyl-L-carnitine (ALCAR) or a salt thereof or a dipeptide or salt thereof, or a tripeptide or salt thereof, comprising ALCAR; wherein at least one amino acid entity (e.g., two, three, four, five, six, or seven amino acid entities) of (a)-(c) is not provided as a peptide of more than 20 amino acid residues in length. In some embodiments, the composition further comprises an aspartate amino acid entity.

In some embodiments, one, two, three, four, five, six, seven, or eight of the leucine amino acid entity, the isoleucine amino acid entity, the valine amino acid entity, the ornithine amino acid entity, the aspartate amino acid entity, the histidine amino acid entity, the lysine amino acid entity, or the threonine amino acid entity is provided as part of a dipeptide (e.g., a homodipeptide or heterodipeptide) or salt thereof. In some embodiments, the leucine amino acid entity is Ala-Leu. In some embodiments, one, two, three, four, five, six, seven, or eight of the leucine amino acid entity, the isoleucine amino acid entity, the valine amino acid entity, the ornithine amino acid entity, the aspartate amino acid entity, the histidine amino acid entity, the lysine amino acid entity, or the threonine amino acid entity is provided as part of a tripeptide (e.g., a homotripeptide or heterotripeptide) or salt thereof.

In some embodiments, the composition is capable of one, two, three, four, five, six, seven, eight, nine, or all (e.g., more) of: a) increasing a level of branched chain amino acids (BCAAs); b) decreasing a level of aromatic amino acids (AAAs); c) decreasing a level of ammonia; d) increasing a level of protein, e.g., increased protein synthesis; e) increasing activation of mTORC1; f) decreasing a level of myostatin; g) decreasing a level of creatinine; h) increasing a level of albumin; i) decreasing a level of bilirubin; j) restoring a Fischer’s ratio (e.g., increasing the level of BCAAs relative to the level of AAAs); or k) increasing a level of valine relative to a level of phenylalanine.

### i. Amounts

An exemplary amino acid composition is amino acid composition J-1. Amino Acid Composition J-1 comprises leucine, isoleucine, valine, N-acetylcysteine, histidine, lysine, and threonine as its defined amino acid components. Amino Acid Composition J-1 is free of the amino acids tyrosine, phenylalanine and glutamine. Example embodiments of these amino acid components in Amino Acid Composition J-1 are shown in Table 3 (grams per packet or unit dosage, grams per day, and weight ratio).

TABLE 3

Amino Acid Components of Composition J-1

| Amino acid | weight ratio | g/daily | g/packet | Daytime dose (2× / day) | Late evening dose (1× / day) |
|---|---|---|---|---|---|
| Leucine | 4 | 12 | 2.0 | 4 | 4 |
| Isoleucine | 2 | 6 | 1.0 | 2 | 2 |
| Valine | 4 | 12 | 2.0 | 4 | 4 |
| N-acetylcysteine | 1 | 3 | 0.5 | 1 | 1 |
| Histidine | 1 | 3 | 0.5 | 1 | 1 |
| Lysine | 1 | 3 | 0.5 | 1 | 1 |
| Threonine | 1 | 3 | 0.5 | 1 | 1 |
| Total amino acids | — | 42 g | 7 g | 14 g | 14 g |
| (Total BCAA) | — | (30 g) | (5 g) | (8 g) | (8 g) |
| Carbohydrate supplement (for nocturnal dosing) | — | — | — | n/a | (>200 kcal) |

In some embodiments, the composition includes 8 g +/- 20% of leucine or the equivalent amount of a leucine amino acid entity, 4 g +/- 20% of isoleucine or the equivalent amount of an isoleucine amino acid entity, 8 g +/- 20% of valine or the equivalent amount of a valine amino acid entity, 3 g +/- 20% of lysine or the equivalent amount of a lysine amino acid entity, 3 g+/-20% of histidine or the equivalent amount of a histidine amino acid entity, 3 g +/- 20% of threonine or the equivalent amount of a threonine amino acid entity, 7.5 g+/- 20% of ornithine or the equivalent amount of an ornithine amino acid entity, 7.5 g+/- 20% of aspartate or the equivalent amount of an aspartate amino acid entity, and 1.5 g+/- 20%, 2.0 g+/- 20%, or 2.5 g+/-20% NAC or the equivalent amount of a NAC entity (see, e.g., grams/day for composition with NAC in Table 4).

TABLE 4

Exemplary composition further comprising NAC and/or L-carnitine

| | Composition with NAC | Composition with L-Carnitine | Composition with NAC and L-Carnitine |
|---|---|---|---|
| Amino Acid | Grams/Day or Wt. Ratio | Grams/Day or Wt. Ratio | Grams/Day or Wt. Ratio |
| L-Leucine | 8 | 8 | 8 |
| L-Isoleucine | 4 | 4 | 4 |
| L-Valine | 8 | 8 | 8 |
| L-Lysine (L-Lysine Acetate) | 3 (4.2 in salt form) | 3 (4.2 in salt form) | 3 (4.2 in salt form) |
| L-Histidine | 3 | 3 | 3 |
| L-Threonine | 3 | 3 | 3 |
| L-Ornithine | 7.5 | 7.5 | 7.5 |
| L-Aspartate | 7.5 | 7.5 | 7.5 |
| N-Acetylcysteine | 1.5-2.5 (e.g., 1.5, 2.0, 2.5) | 0 | 1.5-2.5 (e.g., 1.5, 2.0, 2.5) |
| Carnitine | 0 | 1.0-2.0 (e.g., 1.0, 1.5, 2.0) | 1.0-2.0 (e.g., 1.0, 1.5, 2.0) |
| Total amino acids | 45.5-46.5 | 45-46 | 46.5-48.5 |

In some embodiments, the composition includes 8 g +/- 20% of leucine or the equivalent amount of a leucine amino acid entity, 4 g +/- 20% of isoleucine or the equivalent amount of an isoleucine amino acid entity, 8 g +/- 20% of valine or the equivalent amount of a valine amino acid entity, 3 g +/- 20% of lysine or the equivalent amount of a lysine amino acid entity, 3 g+/-20% of histidine or the equivalent amount of a histidine amino acid entity, 3 g +/- 20% of threonine or the equivalent amount of a threonine amino acid entity, 7.5 g+/- 20% of ornithine or the equivalent amount of an ornithine amino acid entity, 7.5 g+/- 20% of aspartate or the equivalent amount of an aspartate amino acid entity, and 1.0 g+/- 20%, 1.5 g+/- 20%, or 2.0 g+/-20% carnitine or the equivalent amount of a carnitine entity (see, e.g., grams/day for composition with L-carnitine in Table 4).

In some embodiments, the composition includes 8 g +/- 20% of leucine or the equivalent amount of a leucine amino acid entity, 4 g +/- 20% of isoleucine or the equivalent amount of an isoleucine amino acid entity, 8 g +/- 20% of valine or the equivalent amount of a valine amino acid entity, 3 g +/- 20% of lysine or the equivalent amount of a lysine amino acid entity, 3 g+/-20% of histidine or the equivalent amount of a histidine amino acid entity, 3 g +/- 20% of threonine or the equivalent amount of a threonine amino acid entity, 7.5 g+/- 20% of ornithine or the equivalent amount of an ornithine amino acid entity, 7.5 g+/- 20% of aspartate or the equivalent amount of an aspartate amino acid entity, 1.5 g+/- 20%, 2.0 g+/- 20%, or 2.5 g+/-20% NAC or the equivalent amount of a NAC entity, and 1.0 g+/- 20%, 1.5 g+/- 20%, or 2.0 g+/- 20% carnitine or the equivalent amount of a carnitine entity (see, e.g., grams/day for composition with NAC and L-carnitine in Table 4).

### ii. Ratios

In some embodiments, the wt. ratio of the BCAA entity or BCAA entities : the UCAA entity or UCAA entities : the EAA entity or EAA entities in (c) is about 20+/- 20% : 15 +/- 20%: 9+/- 20%, where the ratios are determined based on an equivalent amount of each amino acid in free form. In some embodiments, the wt. ratio of the BCAA entity or BCAA entities : the UCAA entity or UCAA entities : the EAA entity or EAA entities in (c) is about 20+/- 15% : 15 +/- 15%: 9+/- 15%, where the ratios are determined based on an equivalent amount of each amino acid in free form. In some embodiments, the wt. ratio of the BCAA entity or BCAA entities : the UCAA entity or UCAA entities : the EAA entity or EAA entities in (c) is about 10+/- 20% : 15 +/- 10%: 9+/- 10%, where the ratios are determined based on an equivalent amount of each amino acid in free form. In some embodiments, the wt. ratio of the BCAA entity or BCAA entities : the UCAA entity or UCAA entities : the EAA entity or EAA entities in (c) is about 20+/- 15% : 5 +/- 5%: 9+/- 5%, where the ratios are determined based on an equivalent amount of each amino acid in free form.

In some embodiments, the wt. ratio of the leucine amino acid entity : the ornithine amino acid entity : the EAA in (c) is about 8+/- 20% : 7.5+/- 20% : 3+/- 20% or about 8+/- 20% : 7.5+/-20% : 4.2+/- 20%, where the ratios are determined based on an equivalent amount of each amino acid in free form. In some embodiments, the wt. ratio of the leucine amino acid entity : the ornithine amino acid entity : the EAA in (c)about 8+/- 15% : 7.5+/- 15% : 3+/- 15% or about 8+/- 15% : 7.5+/- 15% : 4.2+/- 15%, where the ratios are determined based on an equivalent amount of each amino acid in free form. In some embodiments, the wt. ratio of the leucine amino acid entity : the ornithine amino acid entity : the EAA in (c) is about 8+/- 10% : 7.5+/-10% : 3+/- 10% or about 8+/- 10% : 7.5+/- 10% : 4.2+/- 10%, where the ratios are determined based on an equivalent amount of each amino acid in free form. In some embodiments, the wt. ratio of the leucine amino acid entity : the ornithine amino acid entity : the EAA in (c) is about 8+/- 5% : 7.5+/- 5% : 3+/- 5% or about 8+/- 5% : 7.5+/- 5% : 4.2+/- 5%, where the ratios are determined based on an equivalent amount of each amino acid in free form.

In some embodiments, the wt. ratio of the leucine amino acid entity : the ornithine amino acid entity : the aspartate amino acid entity : the EAA in (c) is about 8+/- 20% : 7.5+/- 20% : 7.5+/- 20% : 3+/- 20% or about 8+/- 20% : 7.5+/- 20% : 7.5+/- 20% : 4.2+/- 20%, where the ratios are determined based on an equivalent amount of each amino acid in free form. In some embodiments, the wt. ratio of the leucine amino acid entity : the ornithine amino acid entity : the aspartate amino acid entity : the EAA in (c) is about 8+/- 15% : 7.5+/- 15% : 7.5+/- 15% : 3+/-15% or about 8+/- 15% : 7.5+/- 15% : 7.5+/- 15% : 4.2+/- 15%, where the ratios are determined based on an equivalent amount of each amino acid in free form. In some embodiments, the wt. ratio of the leucine amino acid entity : the ornithine amino acid entity : the aspartate amino acid entity : the EAA in (c) is about 8+/- 10% : 7.5+/- 10% : 7.5+/- 10% : 3+/- 10% or about 8+/-10% : 7.5+/- 10% : 7.5+/- 10% : 4.2+/- 10%, where the ratios are determined based on an equivalent amount of each amino acid in free form. In some embodiments, the wt. ratio of the leucine amino acid entity : the ornithine amino acid entity : the aspartate amino acid entity : the EAA in (c) is about 8+/- 5% : 7.5+/- 5% : 7.5+/- 5% : 3+/- 5% or about 8+/- 5% : 7.5+/- 5% : 7.5+/- 5% : 4.2+/- 5%, where the ratios are determined based on an equivalent amount of each amino acid in free form.

In some embodiments, the wt. ratio of the leucine amino acid entity : the isoleucine amino acid entity : the valine amino acid entity : the ornithine amino acid entity : the aspartate amino acid entity : the histidine amino acid entity : the threonine amino acid entity : the lysine amino acid entity is 8+/- 20% : 4+/- 20% : 8 +/- 20% : 7.5+/- 20% : 7.5+/- 20% : 3+/- 20% : 3+/-20% : 3+/- 20%, where the ratios are determined based on an equivalent amount of each amino acid in free form. In some embodiments, the wt. ratio of the leucine amino acid entity : the isoleucine amino acid entity : the valine amino acid entity : the ornithine amino acid entity : the aspartate amino acid entity : the histidine amino acid entity : the threonine amino acid entity : the lysine amino acid entity is 8+/- 15% : 4+/- 15% : 8 +/- 15% : 7.5+/- 15% : 7.5+/- 15% : 3+/- 15% : 3+/- 15% : 3+/- 15%, where the ratios are determined based on an equivalent amount of each amino acid in free form. In some embodiments, the wt. ratio of the leucine amino acid entity : the isoleucine amino acid entity : the valine amino acid entity : the ornithine amino acid entity : the aspartate amino acid entity : the histidine amino acid entity : the threonine amino acid entity : the lysine amino acid entity is 8+/- 10% : 4+/- 10% : 8 +/- 10% : 7.5+/- 10% : 7.5+/- 10% : 3+/-10% : 3+/- 10% : 3+/- 10%. In some embodiments, the wt. ratio of the leucine amino acid entity : the isoleucine amino acid entity : the valine amino acid entity : the ornithine amino acid entity : the aspartate amino acid entity : the histidine amino acid entity : the threonine amino acid entity : the lysine amino acid entity is 8+/- 5% : 4+/- 5% : 8 +/- 5% : 7.5+/- 5% : 7.5+/- 5% : 3+/- 5% : 3+/- 5% : 3+/- 5%.

In some embodiments, the wt. ratio of:
(i) the EAA entity or EAA entities (e.g., one, two, or three of a histidine amino acid entity, a lysine amino acid entity, or a threonine amino acid entity) to
(ii) the BCAA entity or BCAA entities (e.g., one, two, or three of a leucine amino acid entity, an isoleucine amino acid entity, or a valine amino acid entity) in combination with the UCAA entity or UCAA entities (e.g., one or both of the ornithine amino acid entity or the aspartate amino acid entity), is at least 1:4 +/- 15%, or at least 1:3 +/- 15%, and not more than 3:4 +/- 15%, e.g., the wt. ratio of of the EAA entity or EAA entities to the BCAA entity or BCAA entities in combination with the UCAA entity or UCAA entities is 1:2 +/- 15%, where the ratios are determined based on an equivalent amount of each amino acid in free form.

In some embodiments, the wt. ratio of:
(i) the histidine amino acid entity, the lysine amino acid entity, and the threonine amino acid entity in combination to
(ii) the leucine amino acid entity, the isoleucine amino acid entity, the valine amino acid entity, the ornithine amino acid entity, and the aspartate amino acid entity in combination is at least 1:4 +/- 15%, or at least 1:3 +/- 15%, and not more than 3:4 +/- 15%, e.g., the wt. ratio of the histidine amino acid entity, the lysine amino acid entity, and the threonine amino acid entity in combination to the leucine amino acid entity, the isoleucine amino acid entity, the valine amino acid entity, the ornithine amino acid entity, and the aspartate amino acid entity in combination is 1:2 +/- 15%, where the ratios are determined based on an equivalent amount of each amino acid in free form.

In some embodiments, the wt. ratio of the UCAA or the combination of two of the UCAA entities to the combination of three of the BCAA entities is at least 5:20 +/- 15%, or at least 10:20 +/- 15%, and not more than 18:20 +/- 15%, e.g., the wt. ratio of the combination of two of the UCAA entities to the combination of three of the BCAA entities is 15:20 +/- 15%, where the ratios are determined based on an equivalent amount of each amino acid in free form.

In some embodiments, the wt. ratio of the combination of three of the EAA entities to the combination of three of the BCAA entities is at least 5:20 +/- 15%, or at least 7:20 +/- 15%, and not more than 15:20 +/- 15%, e.g., the wt. ratio of the combination of three of the EAA entities to the combination of three of the BCAA entities is 9:20+/- 15%, where the ratios are determined based on an equivalent amount of each amino acid in free form.

In some embodiments, the wt. ratio of the combination of three of the EAA entities to the combination of three of the UCAA entities is at least 4:15 +/- 15%, or at least 6:15 +/- 15%, and not more than 13:15 +/- 15%, e.g., the wt. ratio of the combination of three of the EAA entities to the combination of three of the UCAA entities is 9:15 +/- 15%, where the ratios are determined based on an equivalent amount of each amino acid in free form.

In some embodiments, the wt. ratio of the ornithine amino acid entity to the leucine amino acid entity is at least 3:4 +/- 15%, or at least 17:20 +/- 15%, and not more than 5:4 +/-15%, e.g., the wt. ratio of ornithine amino acid entity to the leucine amino acid entity is 15:16 +/-15%, where the ratios are determined based on an equivalent amount of each amino acid in free form.

In some embodiments, the wt. ratio of the EAA entity in (c) to the leucine amino acid entity is at least 1:8 +/- 15%, or least 1:4 +/- 15%, and not more than 3:4 +/- 15%, e.g., the wt. ratio of the EAA entity in (c) to the leucine amino acid entity is 3:8 +/- 15% or 21:40 +/- 15%, where the ratios are determined based on an equivalent amount of each amino acid in free form.

In some embodiments, the wt. ratio of the EAA entity in (c) to the ornithine amino acid entity is at least 2:15 +/- 15%, or least 4:15 +/- 15%, and not more than 2:3 +/- 15%, e.g., the wt. ratio of the EAA entity in (c) to the ornithine amino acid entity is 2:5 +/- 15% or 14:25 +/- 15%, where the ratios are determined based on an equivalent amount of each amino acid in free form.

In some embodiments, the wt. ratio of the EAA entity in (c) to the leucine amino acid entity and the ornithine amino acid entity in combination is at least 2:31 +/- 15%, or least 4:31 +/- 15%, and not more than 12:31 +/- 15%, e.g., the wt. ratio of the EAA entity in (c) to the leucine amino acid entity and the ornithine amino acid entity in combination is 6:31 +/- 15% or 42:155 +/- 15%, where the ratios are determined based on an equivalent amount of each amino acid in free form.

In some embodiments, the wt. ratio of the aspartate amino acid entity to the leucine amino acid entity is at least 3:4 +/- 15%, or at least 17:20 +/- 15%, and not more than 5:4 +/-15%, e.g., the wt. ratio of aspartate amino acid entity to the leucine amino acid entity is 15:16 +/-15%, where the ratios are determined based on an equivalent amount of each amino acid in free form.

In some embodiments, the wt. ratio of the EAA in (c) to the aspartate amino acid entity is at least 2:15 +/- 15%, or least 4:15 +/- 15%, and not more than 4:5 +/- 15%, e.g., the wt. ratio of the EAA in (c) to the aspartate amino acid entity is 2:5 +/- 15% or 14:25 +/- 15%, where the ratios are determined based on an equivalent amount of each amino acid in free form.

In some embodiments, the wt. ratio of the combination of two or three of the EAAs in (c) to the leucine amino acid entity and the aspartate amino acid entity in combination is at least 4:31 +/- 15%, or 6:31 +/- 15%, and not more than 24:31 +/- 15%, e.g., the wt. ratio of the combination of two or three of the EAAs in (c) to the leucine amino acid entity and the aspartate amino acid entity in combination is 12:31 +/- 15%, 72:155 +/- 15%, or 102:155 +/- 15%, where the ratios are determined based on an equivalent amount of each amino acid in free form.

In some embodiments, the wt. ratio of the aspartate amino acid entity to the ornithine amino acid entity is at least 3:4 +/- 15%, or at least 4:5 +/- 15%, and not more than 2:1 +/- 15%, e.g., the wt. ratio of the aspartate amino acid entity to the leucine amino acid entity is 1:1 +/-15%, where the ratios are determined based on an equivalent amount of each amino acid in free form.

In some embodiments, the wt. ratio of the isoleucine amino acid entity to one or both of the leucine amino acid entity or the valine amino acid entity is at least 2:3+/- 15%, or at least 4:7+/- 15%, and not more than 4:5+/- 15%, e.g., the ratio of the isoleucine amino acid entity to one or both of the leucine amino acid entity or the valine amino acid entity is 1:2 +/- 15%, where the ratios are determined based on an equivalent amount of each amino acid in free form.

In some embodiments, the wt. ratio of the isoleucine amino acid entity to one or both of the aspartate amino acid entity or the ornithine amino acid entity is at least 1:3+/- 15%, or at least 3:8+/- 15%, and not more than 3:5+/- 15%, e.g., the ratio of the leucine amino acid entity to one or both of the aspartate amino acid entity or the ornithine amino acid entity is 8:15 +/-15%, where the ratios are determined based on an equivalent amount of each amino acid in free form.

In some embodiments, the wt. ratio of the isoleucine amino acid entity to the combination of two or three of the EAAs in (c) is at least 1 :5+/- 15%, or at least 1 :4+/- 15%, and not more than 3:4+/- 15%, e.g., the ratio of the isoleucine amino acid entity to the combination of two or three of the EAAs in (c) is about 2:3 or about 5:9 or 20:51 +/- 15%, where the ratios are determined based on an equivalent amount of each amino acid in free form.

In some embodiments, the wt. ratio of the ornithine amino acid entity to the valine amino acid entity is at least 3:4+/- 15%, or at least 17:20+/- 15%, and not more than 5:4+/- 15%, e.g., the wt. ratio of ornithine amino acid entity to the valine amino acid entity is 15:16 +/- 15%, where the ratios are determined based on an equivalent amount of each amino acid in free form.

In some embodiments, the wt. ratio of BCAAs to total amino acid entities is at least 1:4+/- 15%, or at least 1:3+/- 15%, and not more than 2:5+/- 15%, e.g., the wt. ratio of ornithine amino acid entity to the valine amino acid entity is 20:44 +/- 15%, where the ratios are determined based on an equivalent amount of each amino acid in free form.

In some embodiments, the wt. ratio of the leucine amino acid entity : the isoleucine amino acid entity : the valine amino acid entity : the lysine amino acid entity : the histidine amino acid entity : the threonine amino acid entity : the ornithine amino acid entity : the aspartate amino acid entity : NAC entity is 8+/- 20% : 4+/- 20% : 8 +/- 20% : 3+/- 20% : 3+/-20% : 3+/- 20% : 7.5+/- 20% : 7.5+/- 20% : 1.5 +/- 20%, where the ratios are determined based on an equivalent amount of each amino acid in free form. In some embodiments, the wt. ratio of the leucine amino acid entity : the isoleucine amino acid entity : the valine amino acid entity : the lysine amino acid entity : the histidine amino acid entity : the threonine amino acid entity : the ornithine amino acid entity : the aspartate amino acid entity : NAC entity is 8+/- 20% : 4+/- 20% : 8 +/- 20% : 3+/- 20% : 3+/- 20% : 3+/- 20% : 7.5+/- 20% : 7.5+/- 20% : 2.0 +/- 20%, where the ratios are determined based on an equivalent amount of each amino acid in free form. In some embodiments, the wt. ratio of the leucine amino acid entity : the isoleucine amino acid entity : the valine amino acid entity : the lysine amino acid entity : the histidine amino acid entity : the threonine amino acid entity : the ornithine amino acid entity : the aspartate amino acid entity : NAC entity is 8+/- 20% : 4+/- 20% : 8 +/- 20% : 3+/- 20% : 3+/- 20% : 3+/- 20% : 7.5+/- 20% : 7.5+/- 20% : 2.5 +/- 20%, where the ratios are determined based on an equivalent amount of each amino acid in free form. In some embodiments, the wt. ratio of the leucine amino acid entity : the isoleucine amino acid entity : the valine amino acid entity : the lysine amino acid entity : the histidine amino acid entity : the threonine amino acid entity : the ornithine amino acid entity : the aspartate amino acid entity : carnitine entity is 8+/- 20% : 4+/- 20% : 8 +/- 20% : 3+/- 20% : 3+/-20% : 3+/- 20% : 7.5+/- 20% : 7.5+/- 20% : 1.0 +/- 20%, where the ratios are determined based on an equivalent amount of each amino acid in free form. In some embodiments, the wt. ratio of the leucine amino acid entity : the isoleucine amino acid entity : the valine amino acid entity : the lysine amino acid entity : the histidine amino acid entity : the threonine amino acid entity : the ornithine amino acid entity : the aspartate amino acid entity : carnitine entity is 8+/- 20% : 4+/-20% : 8 +/- 20% : 3+/- 20% : 3+/- 20% : 3+/- 20% : 7.5+/- 20% : 7.5+/- 20% : 1.5 +/- 20%, where the ratios are determined based on an equivalent amount of each amino acid in free form. In some embodiments, the wt. ratio of the leucine amino acid entity : the isoleucine amino acid entity : the valine amino acid entity : the lysine amino acid entity : the histidine amino acid entity : the threonine amino acid entity : the ornithine amino acid entity : the aspartate amino acid entity : carnitine entity is 8+/- 20% : 4+/- 20% : 8 +/- 20% : 3+/- 20% : 3+/- 20% : 3+/- 20% : 7.5+/- 20% : 7.5+/- 20% : 2.0 +/- 20%, where the ratios are determined based on an equivalent amount of each amino acid in free form.

In some embodiments, the wt. ratio of the leucine amino acid entity : the isoleucine amino acid entity : the valine amino acid entity : the lysine amino acid entity : the histidine amino acid entity : the threonine amino acid entity : the ornithine amino acid entity : the aspartate amino acid entity : NAC entity : carnitine entity is 8+/- 20% : 4+/- 20% : 8 +/- 20% : 3+/- 20% : 3+/- 20% : 3+/- 20% : 7.5+/- 20% : 7.5+/- 20% : 1.5 +/- 20% : 2.0 +/- 20%, where the ratios are determined based on an equivalent amount of each amino acid in free form. In some embodiments, the wt. ratio of the leucine amino acid entity : the isoleucine amino acid entity : the valine amino acid entity : the lysine amino acid entity : the histidine amino acid entity : the threonine amino acid entity : the ornithine amino acid entity : the aspartate amino acid entity : NAC entity : carnitine entity is 8+/- 20% : 4+/- 20% : 8 +/- 20% : 3+/- 20% : 3+/- 20% : 3+/-20% : 7.5+/- 20% : 7.5+/- 20% : 2.0 +/- 20% : 1.5 +/- 20%, where the ratios are determined based on an equivalent amount of each amino acid in free form. In some embodiments, the wt. ratio of the leucine amino acid entity : the isoleucine amino acid entity : the valine amino acid entity : the lysine amino acid entity : the histidine amino acid entity : the threonine amino acid entity : the ornithine amino acid entity : the aspartate amino acid entity : NAC entity : carnitine entity is 8+/- 20% : 4+/- 20% : 8 +/- 20% : 3+/- 20% : 3+/- 20% : 3+/- 20% : 7.5+/- 20% : 7.5+/-20% : 2.5 +/- 20% : 1.0 +/- 20%, where the ratios are determined based on an equivalent amount of each amino acid in free form.

### iii. Relationships of Amino Acid Entities

In some embodiments, the wt. % of one, two in combination, or three in combination of the BCAA entities is greater than the wt. % of one or two in combination of the UCAA entities, e.g., the wt. % of one, two in combination, or three in combination of the BCAA entities is at least 5% greater than the wt. % of one or two in combination of the UCAA entities; e.g., the wt. % of one, two in combination, or three in combination of the BCAA entities is at least 10%, 15%, 20%, 25%, or 30% greater than the wt. % of one or two in combination of the UCAA entities.

In some embodiments, the wt. % of one, two in combination, or three in combination of the BCAA entities is greater than the wt. % of one, two in combination, or three in combination of the EAA entities in (c); e.g., the wt. % of one, two in combination, or three in combination of the BCAA entities is at least 50% greater than the wt. % of one, two in combination, or three in combination of the EAA entities in (c); e.g., the wt. % of one, two in combination, or three in combination of the BCAA entities is at least 60%, 70%, 80%, 90%, or 100% greater than the wt. % of one, two in combination, or three in combination of the EAA entities in (c).

In some embodiments, the wt. % of one or two in combination of the UCAA entities is greater than the wt. % of one, two in combination, or three in combination of the EAA entities in (c); e.g., the wt. % of one or two in combination of the UCAA entities is at least 25% greater than the wt. % of one, two in combination, or three in combination of the EAA entities in (c); e.g., the wt. % of one or two in combination of the UCAA entities is at least 30%, 45%, 50%, 55%, or 60% greater than the wt. % of one, two in combination, or three in combination of the EAA entities in (c).

In some embodiments, the wt. % of:
(i) the BCAA entity or BCAA entities (e.g., one, two, or three of a leucine amino acid entity, an isoleucine amino acid entity, or a valine amino acid entity) in combination with the UCAA entity or UCAA entities (e.g., one or both of an ornithine amino acid entity or an aspartate amino acid entity) is greater than
(ii) the wt. % of the EAA entity or EAA entities (e.g., one, two, or three of a histidine amino acid entity, a lysine amino acid entity, or a threonine amino acid entity);
e.g., the wt. % of the BCAA entity or BCAA entities in combination with the UCAA entity or UCAA entities is at least 50% greater than the wt. % of the EAA entity or EAA entities; e.g., the wt. % of the BCAA entity or BCAA entities in combination with the UCAA entity or UCAA entities is at least 60%, 70%, 80%, or 90% greater than the wt. % of the EAA entity or EAA entities.

In some embodiments, the wt. % of:
(i) the leucine amino acid entity, the isoleucine amino acid entity, the valine amino acid entity, the ornithine amino acid entity, and the aspartate amino acid entity in combination is greater than:
(ii) the wt. % of the histidine amino acid entity, the lysine amino acid entity, and the threonine amino acid entity in combination;
e.g., the wt. % of:
(i) the leucine amino acid entity, the isoleucine amino acid entity, the valine amino acid entity, the ornithine amino acid entity, and the aspartate amino acid entity in combination is at least 50% greater than:
(ii) the wt. % of the histidine amino acid entity, the lysine amino acid entity, and the threonine amino acid entity in combination;
e.g., the wt. % of:
(i) the leucine amino acid entity, the isoleucine amino acid entity, the valine amino acid entity, the ornithine amino acid entity, and the aspartate amino acid entity in combination is at least 60%, 70%, 80%, or 90% greater than:
(ii) the wt. % of the histidine amino acid entity, the lysine amino acid entity, and the threonine amino acid entity in combination.

In some embodiments, the wt. % of one or both of the leucine amino acid entity or the valine amino acid entity is greater than the wt. % of one or both of the ornithine amino acid entity or the aspartate amino acid entity, e.g., the wt. % of one or both of the leucine amino acid entity or the valine amino acid entity is at least 2% greater than the wt. % of one or both of the ornithine amino acid entity or the aspartate amino acid entity, e.g., the wt. % of one or both of the leucine amino acid entity or the valine amino acid entity is at least 3%, 4%, 5%, or 6% greater than the wt. % of one or both of the ornithine amino acid entity or the aspartate amino acid entity.

In some embodiments, the wt. % of one or both of the leucine amino acid entity or the valine amino acid entity is greater than the wt. % of the EAA entity or the combination of two EAA entities in (c), e.g., the wt. % of one or both of the leucine amino acid entity or the valine amino acid entity is at least 10% greater than the wt. % of the EAA entity or the combination of two EAA entities in (c), e.g., the wt. % of one or both of the leucine amino acid entity or the valine amino acid entity is at least 12%, 15%, 20%, 22%, or 25% greater than the wt. % of the EAA entity or the combination of two EAA entities in (c).

In some embodiments, the wt. % of one or both of the ornithine amino acid entity and the aspartate amino acid entity is greater than the wt. % of the EAA entity or the combination of two EAA entities in (c), e.g., the wt. % of one or both of the ornithine amino acid entity and the aspartate amino acid entity is at least 4% greater than the wt. % of the EAA entity or the combination of two EAA entities in (c), e.g., the wt. % of one or both of the ornithine amino acid entity and the aspartate amino acid entity is at least 5%, 10%, 15%, 20%, or 25% greater than the wt. % of the EAA entity or the combination of two EAA entities in (c).

In some embodiments, the wt. % of one or both of the aspartate amino acid entity or the ornithine amino acid entity is greater than the isoleucine amino acid entity, e.g., the wt. % of one or both of the aspartate amino acid entity or the ornithine amino acid entity is at least 65% greater than the wt. % of the isoleucine amino acid entity, e.g., the wt. % of one or both of the aspartate amino acid entity or the ornithine amino acid entity is at least 70%, 75%, 80%, or 85% greater than the wt. % of the isoleucine amino acid entity.

In some embodiments, the wt. % of the leucine amino acid entity or the valine amino acid entity and the ornithine amino acid entity or the aspartate amino acid entity in combination in (a) and (b) is greater than the wt. % of the EAA entity or a combination of two or three of the EAA entities in (c), e.g., the wt. % of the leucine amino acid entity or the valine amino acid entity and the ornithine amino acid entity or the aspartate amino acid entity in combination is at least 20% greater than the wt. % of the EAA entity or the combination of two or three of the EAA entities in (c), e.g., the wt. % of the leucine amino acid entity or the valine amino acid entity and the ornithine amino acid entity or the aspartate amino acid entity in combination is at least 25%, 30%, 35%, 40%, or 50% greater than the wt. % of the EAA entity, or a combination of two or three of the EAA entities in (c).

In some embodiments, the wt. % of one or both of the leucine amino acid entity or the valine amino acid entity is greater than the wt. % of one or both of the aspartate amino acid entity or the ornithine amino acid entity, e.g., the wt. % of one or both of the leucine amino acid entity or the valine amino acid entity is at least 2% greater than the wt. % of the aspartate amino acid entity or the ornithine amino acid entity, e.g., the wt. % of one or both of the leucine amino acid entity or the valine amino acid entity is at least 3%, 4%, 5%, or 6% greater than the wt. % of the aspartate amino acid entity or the ornithine amino acid entity.

In some embodiments, the wt. % of one or both of the aspartate amino acid entity or the ornithine amino acid entity is greater than the wt. % of one or two of the EAA entities in (c), e.g., the wt. % of one or both of the aspartate amino acid entity or the ornithine amino acid entity is at least 15% greater than the wt. % of one or two of the EAA entities in (c), e.g., the wt. % of one or both of the aspartate amino acid entity or the ornithine amino acid entity is at least 20%, 25%, 30%, or 35% greater than the wt. % of one or two of the EAA entities in (c).

In some embodiments, the wt. % of the leucine amino acid entity and the aspartate amino acid entity in combination is greater than the wt. % of the EAA, or the combination of two or three of the EAAs in (c), e.g., the wt. % of the leucine amino acid entity and the aspartate amino acid entity in combination is at least 20% greater than the wt. % of the EAA, or the combination of two or three of the EAAs in (c), e.g., the wt. % of the leucine amino acid entity and the aspartate amino acid entity in combination is at least 25%, 30%, 35%, 40%, or 50% greater than the wt. % of the EAA, or the combination of two or three of the EAAs in (c);

In some embodiments, the wt. % of the leucine amino acid entity, the isoleucine amino acid entity, and the valine amino acid entity in combination is at least 20%, at least 30%, or at least 40 % of the composition, but not more than 70% of the composition. In some embodiments, the wt. % of the ornithine amino acid entity and the aspartate amino acid entity in combination is at least 15%, at least 25%, or at least 35 % of the composition, but not more than 60% of the composition.

In some embodiments, the wt. % of one or both of the leucine amino acid entity or valine amino acid entity is greater than the isoleucine amino acid entity, e.g., the wt. % of one or both of the leucine amino acid entity or valine amino acid entity is at least 25% greater than the wt. % of the isoleucine amino acid entity, e.g., the wt. % of one or both of the leucine amino acid entity orvaline amino acid entity is at least 30%, 35%, 40%, or 45% greater than the wt. % of the isoleucine amino acid entity. In some embodiments, the wt. % of the leucine amino acid entity is equal to wt. % the valine amino acid entity in the composition.

In some embodiments, the wt. % of the combination of two or three of the EAAs in (c) is greater than the isoleucine amino acid entity, e.g., the wt. % of the combination of two or three of the EAAs in (c) is at least 25% greater than the wt. % of the isoleucine amino acid entity, e.g., the wt. % of the combination of two or three of the EAAs in (c) is at least 30%, 35%, 45%, or 50% greater than the wt. % of the isoleucine amino acid entity.

In some embodiments, the BCAA entity or BCAA entities (e.g., one, two, or three of a leucine amino acid entity, an isoleucine amino acid entity, or a valine amino acid entity) in combination with the UCAA entity or UCAA entities (e.g., one or both of an ornithine amino acid entity or an aspartate amino acid entity) is present at an amount of at least 50% +/- 15%, e.g.,at least 50% +/- 15% to 66% +/- 15%, of the total wt. of amino acid entities.

In some embodiments, the EAA entity or EAA entities (e.g., one, two, or three of a histidine amino acid entity, a lysine amino acid entity, or a threonine amino acid entity) is present at an amount of at most 20% +/- 15%, e.g., at most 20% +/- 15% to 33% +/- 15%, of the total wt. of amino acid entities.

In some embodiments, the leucine amino acid entity, the isoleucine amino acid entity, the valine amino acid entity, the ornithine amino acid entity, and the aspartate amino acid entity in combination is present at an amount of at least 50% +/- 15%, e.g.,at least 50% +/- 15% to 66% +/- 15%, of the total wt. of amino acid entities.

In some embodiments, the histidine amino acid entity, the lysine amino acid entity, and the threonine amino acid entity is present at an amount of at most 20% +/- 15%, e.g., at most 20% +/- 15% to 33% +/- 15%, of the total wt. of amino acid entities.

In some embodiments, one or both of the leucine amino acid entity or the valine amino acid entity is present at 10% +/- 15% to 30% +/- 15% of the total wt. of amino acid entities, e.g., 18.2 %+/- 15%. In some embodiments, the valine amino acid entity is present at 12% +/- 15% to 30% +/- 15% of the total wt. of amino acid entities, e.g., 18.2 %+/- 15%. In some embodiments, the leucine amino acid entity is present at 10% +/- 15% to 25% +/- 15% of the total wt. of amino acid entities, e.g., 18.2 %+/- 15%.

In some embodiments, the isoleucine amino acid entity is present at 5% +/- 15% to 20%+/- 15% of the total wt. of amino acid entities, e.g., 9.1 % +/- 15%. In some embodiments, one or both of the ornithine amino acid entity or the aspartate amino acid entity is each present at 10% +/- 15% to 30% +/- 15% of the total wt. of amino acid entities, e.g., 17.1 % +/- 15% (e.g., the combination of ornithine amino acid entity and the aspartate amino acid entity are present at 17.1% +/- 15% of the total wt. of amino acid entities). In some embodiments, one, two, or three of the the histidine amino acid entity, the threonine amino acid entity, or the lysine amino acid entity are each present at 2% +/- 15% to 15% +/- 15% of the total wt. of amino acid entities, e.g., 6.8% +/- 15%.

### iv. Molecules to Exclude or Limit from the Composition

In some embodiments, the composition does not comprise a peptide of more than 20 amino acid residues in length (e.g., protein supplement) chosen from or derived from one, two, three, four, five, six, seven, eight, nine, 10, 11, 12, 13, 14, 15, or more (e.g., all) of egg white protein, soy protein, milk protein, casein, caseinate, hemp protein, pea protein, wheat protein, oat protein, spirulina, microprotein, lentil protein, quinoa protein, lentil protein, beef protein, or brown rice protein, or if the peptide is present, the peptide is present at less than: 10 weight (wt.) 5 wt. %, 1 wt. %, 0.1 wt. %, 0.05 wt. %, 0.01 wt. %,of the total wt. of amino acid entities or total components in the composition (in dry form).

In some embodiments, the composition comprises a combination of 3 to 19, 3 to 18, 3 to 16, 3 to 15, or 3 to 10 different amino acid entities, e.g., the combination comprises at least: 42 wt. %, 75 wt. %, or 90 wt. % of the total wt. % of amino acid entities or total components in the composition (in dry form).

In some embodiments, dipeptides or salts thereof or tripeptides or salts thereof are present at less than: 10 wt. %, 0.5 wt. %, 0.1 wt. %, 0.05 wt. %, 0.01 wt. %, 0.001 wt. %, or less of the total wt. of amino acid entities or total components in the composition (in dry form).

In some embodiments, at least 50%, 60%, 70%, or more of the total grams of amino acid entities or total components in the composition (in dry form) are from one, two, three, four, five, or more (e.g., all) of (a)-(c).

In some embodiments, at least: 50%, 60%, 70%, or more of the calories from amino acid entities or total components in the composition (in dry form) are from three, four, five, six, seven, or eight of the amino acid entities in (a)-(c).

In some embodiments, one, two, or three of the EAA entities is not an aromatic amino acid (AAA), or if the AAA is present in the composition, the AAA is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form). In some embodiments, the AAA is one or both of phenylalanine or tyrosine. In some embodiments, phenylalanine is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form). In some embodiments, tyrosine is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, glutamine is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, methionine is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form). In some embodiments, proline is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form). In some embodiments, tryptophan is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form). In some embodiments, one, two, or three of methionine, proline, or tryptophan is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, arginine is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form). In some embodiments, glycine is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form). In some embodiments, arginine and glycine are absent from the composition, or if present, are present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, a carbohydrate (e.g., one, two, three, four, five, six, seven, eight, nine, 10, 11, 12, 13, 14, 15, 16, 17, or 18 of dextrose, maltodextrose, sucrose, dextrin, fructose, galactose, glucose, glycogen, high fructose corn syrup, honey, inositol, invert sugar, lactose, levulose, maltose, molasses, sugarcane, or xylose) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, a vitamin (e.g., one, two, three, four, five, six, or seven of vitamin B1, vitamin B2, vitamin B3, vitamin B6, vitamin B 12, vitamin C, or vitamin D) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, one or both of nitrate or nitrite are absent from the composition, or if present, are present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, 4-hydroxyisoleucine is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, a probiotic (e.g., a Bacillus probiotic) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, phenylacetate is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, gelatin (e.g., a gelatin capsule) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

### Use of the Combination, e.g., Methods of Treatment

The disclosure provides a method for improving liver function, comprising administering to a subject in need thereof an effective amount of a combination disclosed herein. The combination can be administered according to a dosage regimen described herein to improve liver function in a subject (e.g., a human). The disclosure provides a method for treating or preventing a liver disease or disorder in a subject, comprising administering to a subject in need thereof an effective amount of a combination disclosed herein. The combination can be administered according to a dosage regimen described herein to treat a liver disease or disorder in a subject (e.g. a human).

In some embodiments, the subject has been diagnosed with a liver disease or disorder. In some embodiments, the subject has not been diagnosed with a liver disease or disorder. In some embodiments, the subject is a human. In some embodiments, the subject has not received prior treatment with one or both of the composition comprising amino acid entities (e.g., an Active Moiety) or one or more of the therapeutic agents described herein (e.g., a naive subject).

In some embodiments, the combination described herein is for use as a medicament in improving liver function in a subject (e.g., a subject with a liver disease or disorder). In some embodiments, the combination is for use as a medicament in treating (e.g., reversing, reducing, ameliorating, or preventing) a disease (e.g., a liver disease or disorder) in a subject. In some embodiments, the combination described herein is for use in the manufacture of a medicament for improving liver function in a subject (e.g., a subject with a liver disease or disorder). In some embodiments, the combination is for use in the manufacture of a medicament for treating (e.g., reversing, reducing, ameliorating, or preventing) a disease (e.g., a liver disease or disorder) in a subj ect.

A subject that may be treated with the combination described herein includes a subject having a fatty liver disease or disorder. In some embodiments, the fatty liver disease or disorder is chosen from: non-alcoholic fatty liver disease (NAFLD) or alcoholic fatty liver disease (AFLD). In certain embodiments, the NAFLD is chosen from: non-alcoholic steatohepatitis (NASH) or non-alcoholic fatty liver (NAFL). In certain embodiments, the subject (e.g., a child or an adolescent) has pediatric NAFLD. In certain embodiments, the AFLD is alcoholic steatohepatitis (ASH).

In some embodiments, the subject has one or both of fibrosis or steatosis. In certain embodiments, the subject (e.g., a subject with NASH) has cirrhosis. In some embodiments, the subject has hepatocarcinoma. In certain embodiments, the subject has one or both of an increased risk of liver failure or an increased risk of death. In some embodiments, the subject has one, two, three, or more (e.g., all) of diabetes (e.g., type 2 diabetes), metabolic syndrome, a relatively high BMI, or obesity. In some embodiments, the subject has one, two, or more (e.g., all) of gut leakiness, gut dysbiosis, or gut microbiome disturbance.

In some embodiments, the subject exhibits a symptom of a liver disease or disorder, e.g., a metabolic symptom. In some embodiments, a subject exhibits a metabolic symptom of liver disease chosen from one, two, three, four, five, six, seven, eight, nine, ten, eleven, or more (e.g., all) of: decreased fat metabolism, hepatocyte apoptosis, hepatocyte ballooning, inflammation of adipose tissue, inflammation of hepatic tissue, fibrosis, liver injury, steatosis, oxidative stress (e.g., one, two, or more (e.g., all) of increased levels of reactive oxygen species (ROS), decreased mitochondrial function, or decreased levels of glutathionine (GSH)), decreased gut barrier function, decreased insulin secretion, or decreased glucose tolerance (e.g., relative to a healthy subject without a liver disease).

In some embodiments, administration of the combination results in an improvement in a metabolic symptom of liver disease in a subject chosen from one, two, three, four, five, six, seven, eight, nine, ten, eleven, twelve, thirteen, fourteen, fifteen, or more (e.g., all) of: increased free fatty acid and lipid metabolism (e.g., in the liver); white adipose tissue (WAT) browning; a reduction in liver fat; decreased hepatocyte apoptosis; decreased hepatocyte ballooning; decreased inflammation of adipose tissue; decreased inflammation of hepatic tissue; a reduction or inhibition of fibrosis; healing of liver injury; decreased steatosis; decreased reactive oxygen species (ROS); improved mitochondrial function; increased levels of glutathione (GSH); improved gut barrier function; increased insulin secretion; or improved glucose tolerance.

In some embodiments, administration of the combination described herein to a subject reduces the level or activity of a pro-inflammatory cytokine (e.g., one, two, three, or more (e.g., all) of TNFα, IL-1, IL-6, or IFNγ), e.g., relative to a normal subject without a fatty liver disease. In some embodiments, administration of the combination described herein to a subject reduces the level or activity of a pro-inflammatory mediator (e.g., NF-kB), e.g., relative to a normal subject without a fatty liver disease. In some embodiments, administration of the combination described herein to a subject increases the level or activity of a anti-inflammatory cytokine (e.g., one, two, three, or more (e.g., all) of IL-10, IL-4, IL-13, and IL-5), e.g., relative to a normal subject without a fatty liver disease. In some embodiments, administration of the combination reduces liver enzyme levels (e.g., one or both of ALT or AST) in one or both of blood or plasma from a subject (e.g., a subject with fatty liver disease), e.g., relative to the subject prior to administration of the combination.

The disclosure provides a method for treating a subject with a diabetic condition, comprising administering to a subject in need thereof an effective amount of a combination disclosed herein, thereby treating the subject. In some embodiments, the subject has diabetic peripheral neuropathy. In certain embodiments, the subject exhibits a symptom of a diabetic condition (e.g., diabetic peripheral neuropathy) chosen from one, two, three, four, five, six, seven, eight, nine, ten, eleven, twelve, thirteen, fourteen, or more (e.g., all) of: trouble with balance; numbness of extremities; tingling of extremities; dysesthesia; diarrhea; erectile dysfunction; loss of bladder control; facial, mouth, or eyelid drooping; vision change; dizziness; muscle weakness; difficulty swallowing; speech impairment; fasciculation; or burning or electric pain. In certain embodiments, administration of the combination to the subject results in an improvement in a symptom of a diabetic condition (e.g., diabetic peripheral neuropathy) chosen from one, two, three, four, five, six, seven, eight, nine, ten, eleven, twelve, thirteen, fourteen, or more (e.g., all) trouble with balance; numbness of extremities; tingling of extremities; dysesthesia; diarrhea; erectile dysfunction; loss of bladder control; facial, mouth, or eyelid drooping; vision change; dizziness; muscle weakness; difficulty swallowing; speech impairment; fasciculation; or burning or electric pain.

### Administration of the Combination

The invention features the administration of a composition comprising amino acid entities (e.g., an Active Moiety) and one or more therapeutic agents in combination (e.g., in one or more compositions or dosage forms). The combinations disclosed herein can be administered together in a single composition or administered separately in two or more different compositions, e.g., compositions or dosage forms as described herein.

Administration of the one or more therapeutic agents and composition comprising amino acid entities (e.g., an Active Moiety) in combination can be in any order. In some embodiments, the composition comprising amino acid entities is administered prior to the one or more therapeutic agents. In some embodiments, the composition comprising amino acid entities is administered subsequent to the one or more therapeutic agents. In some embodiments, the composition comprising amino acid entities is administered concurrently with the one or more therapeutic agents. In some embodiments, the one or more therapeutic agents is administered prior to the composition comprising amino acid entities. In some embodiments, the one or more therapeutic agents is administered subsequent to the composition comprising amino acid entities. In some embodiments, the one or more therapeutic agents is administered concurrently with the composition comprising amino acid entities. In certain embodiments, the composition comprising amino acid entities is administered at the same time on the same day as the therapeutic agent in a therapeutic regimen. In certain embodiments, the composition comprising amino acid entities is administered at a different time of day on the same day as the therapeutic agent in a therapeutic regimen in a therapeutic regimen. In certain embodiments, the composition comprising amino acid entities is administered on a different day than the therapeutic agent in a therapeutic regimen.

When administered in combination, one or both of the composition comprising amino acid entities (e.g., an Active Moiety), or the one or more therapeutic agents can be administered in an amount or dose that is higher, lower, or the same than the amount or dosage of each agent used individually, e.g., as a monotherapy. In some embodiments, one or both of the composition comprising amino acid entities, or the one or more therapeutic agents is administered at a therapeutic or lower-than therapeutic dose. In certain embodiments, the administered amount or dosage of one or both of the composition comprising amino acid entities, or the one or more therapeutic agents is lower (e.g., at least 20%, at least 30%, at least 40%, or at least 50%) than the amount or dosage of each used individually, e.g., as a monotherapy. In other embodiments, the amount or dosage of the composition comprising amino acid entities or the one or more therapeutic agents that results in a desired effect (e.g., treatment of a liver disease or disorder) is lower (e.g., at least 20%, at least 30%, at least 40%, or at least 50% lower).

The composition comprising amino acid entities (e.g., an Active Moiety) and the one or more therapeutic agents may be provided to a subject (e.g., a subject with a liver disease or disorder) in either a single or multiple dosage regimen. Administration may be one or multiple times daily, weekly (or at some other multiple day interval) or on an intermittent schedule, with that cycle repeated a given number of times (e.g., 2, 3, 4, 5, 6,7, 8, 9, 10, or more cycles) or indefinitely. In some embodiments, one or both of the composition comprising amino acid entities or the one or more therapeutic agents is administered twice daily, three times daily, four times daily, five times daily, six times daily, seven times daily, or more, e.g., at the same time or a different times of the day. In certain embodiments, one or both of the composition comprising amino acid entities or the one or more therapeutic agents is administered one, two, or three times daily. One or both of the composition comprising amino acid entities or the one or more therapeutic agents can be administered every 2 hours, every 3 hours, every 4 hours, every 5 hours, every 6 hours, every 7 hours, every 8 hours, every 9 hours, or every 10 hours.

In some embodiments, one or both of the composition comprising amino acid entities (e.g., an Active Moiety) or the one or more therapeutic agents may be administered to a subject (e.g., a subject with a liver disease or disorder) for a treatment period of, e.g., two weeks, three weeks, four weeks, five weeks, six weeks, seven weeks, eight weeks, nine weeks, 10 weeks, 11 weeks, 12 weeks, 13 weeks, 14 weeks, 15 weeks, 16 weeks, or longer. In some embodiments, one or both of the composition comprising amino acid entities or the one or more therapeutic agents is administered for at least 2 days, 3 days, 4 days, 5 days, 6 days, 7 days, or 2 weeks. In some embodiments, one or both of the composition comprising amino acid entities or the one or more therapeutic agents is administered chronically (e.g., more than 30 days, e.g., 31 days, 40 days, 50 days, 60 days, 3 months, 6 months, 9 months, one year, two years, or three years).

In some embodiments, one or both of the composition comprising amino acid entities (e.g., an Active Moiety) or the one or more therapeutic agents is administered prior to a meal. In other embodiments, one or both of the composition comprising amino acid entities or the one or more therapeutic agents is administered concurrent with a meal. In other embodiments, one or both of the composition comprising amino acid entities or the one or more therapeutic agents is administered following a meal.

In some embodiments, the composition comprising amino acid entities (e.g., an Active Moiety) is packaged in one stick pack, two stick packs, three stick packs, four stick packs, five stick packs, six stick packs, or more stick packs. An exemplary composition comprises three stick packs, e.g., each stick pack comprising 33.3% +/- 15% of the quantity of each amino acid entity included in the composition described herein. In certain embodiments, three stick packs are administered two times daily. In a specific embodiment, exemplified herein, three stick packs make up a single dose. An exemplary composition comprises one stick pack or one sachet comprising a dose of the composition comprising amino acids.

In some embodiments, the composition comprising amino acid entities (e.g., an Active Moiety) is administered at a dose of 2 g+/- 20% g daily to 80 g+/- 20% g daily (e.g., 40 g +/-20% total amino acid entities daily). In certain embodiments, the composition is administered at a dose of 30 g +/- 20% to 60 g +/- 20% total amino acid entities three times daily, e.g., 40 g +/-20% total amino acid entities daily. In some embodiments, the composition is administered at a dose of about 2 g +/- 20% to 60 g +/- 20% total amino acid entities, e.g., once daily, twice daily, three times daily, four times daily, five times daily, or six times daily (e.g., twice daily). In some embodiments, the composition is administered at a dose of 2 g +/- 20% to 10 g +/- 20%, 10 g +/-20% to 30 g +/- 20%, or 30 g +/- 20% to 60 g +/- 20% total amino acid entities, e.g., once daily, twice daily, or three times daily (e.g., twice per day). In certain embodiments, the composition is administered at a dose of 10 g +/- 20% to 30 g +/- 20% total amino acid entities twice daily, e.g., 20 g +/- 20% total amino acid entities twice daily.

### Methods of Treatment

The disclosure provides a method for improving one, two, three, or more (e.g., all) of liver function, hyperammonemia, muscle mass, or muscle function, comprising administering to a subject in need thereof an effective amount of a composition disclosed herein (e.g., an Active Moiety). The composition can be administered according to a dosage regimen described herein to improve one, two, three, or more (e.g., all) of liver function, hyperammonemia, muscle mass, or muscle function in a subject (e.g., a human).

The disclosure provides a method for treating or preventing a liver disease or disorder with one or both of hyperammonemia or muscle wasting (e.g., cirrhosis, e.g., cirrhotic sarcopenia, End Stage Liver Disease, hepatic insufficiency, hepatic encephalopathy, or a combination thereof), comprising administering to a subject in need thereof an effective amount of a composition disclosed herein (e.g., an Active Moiety). The composition can be administered according to a dosage regimen described herein to treat a liver disease or disorder with one or both of hyperammonemia or muscle wasting in a subject (e.g. a human).

In some embodiments, the subject has been diagnosed with a liver disease or disorder with one or both of hyperammonemia or muscle wasting (e.g., cirrhosis, e.g., cirrhotic sarcopenia, End Stage Liver Disease, hepatic insufficiency, hepatic encephalopathy, or a combination thereof). In some embodiments, the subject has not been diagnosed with a liver disease or disorder with one or both of hyperammonemia or muscle wasting. In some embodiments, the subject is a human. In some embodiments, the subject has not received prior treatment with the composition described herein (e.g., a naive subject).

In some embodiments, the composition described herein (e.g., the Active Moiety) is for use as a medicament in improving one, two, three, or more (e.g., all) of liver function, hyperammonemia, muscle mass, or muscle function in a subject (e.g., a subject with a liver disease or disorder with one or both of hyperammonemia or muscle wasting). In some embodiments, the composition is for use as a medicament in treating (e.g., reversing, reducing, ameliorating, or preventing) a liver disease or disorder with one or both of hyperammonemia or muscle wasting in a subject.

In some embodiments, the composition described herein (e.g., the Active Moiety) is for use in the manufacture of a medicament for improving one, two, three, or more (e.g., all) of liver function, hyperammonemia, muscle mass, or muscle function in a subject (e.g., a subject with a liver disease or disorder with one or both of hyperammonemia or muscle wasting). In some embodiments, the composition (e.g., the Active Moiety) is for use in the manufacture of a medicament for treating (e.g., reversing, reducing, ameliorating, or preventing) a liver disease or disorder with one or both of hyperammonemia or muscle wasting in a subject.

In some embodiments of any of the aspects or embodiments disclosed herein, the subject has muscle wasting. In some embodiments of any of the aspects or embodiments disclosed herein, the subject has hyperammonemia.

A subject that may be treated with the composition described herein (e.g., the Active Moiety) includes a subject having cirrhosis. In some embodiments, a subject with cirrhosis has cirrhotic sarcopenia, End Stage Liver Disease, hepatic insufficiency, hepatic encephalopathy, or a combination thereof. In some embodiments, the subject has cirrhotic sarcopenia. In some embodiments, the subject has End Stage Liver Disease. In some embodiments, the subject has hepatic insufficiency. In some embodiments, the subject has hepatic encephalopathy.

In some embodiments, the subject has a metabolic symptom chosen from one, two, three, four, five, six, seven, or more (e.g., all) of increased ammonia levels (e.g., hyperammonemia), decreased levels of branched chain amino acids (BCAAs), increased levels of aromatic AAs (AAAs), hypercatabolism, decreased protein synthesis (e.g., a decreased fractional synthesis rate (FSR), e.g., in one or both of muscle or liver tissue), increased reactive oxygen species (ROS), decreased anabolism, or increased autophagy (e.g., relative to a healthy subject without a liver disease or disorder). In some embodiments, a level of one, two, or more (e.g., all) of ammonia, BCAAs, or AAs are measured in a plasma sample from the subject. In some embodiments, overnight fasting exacerbates catabolism in the subject, e.g., prior to treatment with a composition described herein (e.g., a composition including a carbohydrate supplement). In some embodiments, the method further includes monitoring the subject for an improvement in the metabolic symptom.

In some embodiments, a level (e.g., in a plasma sample) of one, two, or more (e.g., all) of L-valine, L-leucine, or L-isoleucine is decreased in the subject, e.g., prior to treatment with a composition described herein (e.g., relative to a healthy subject without a liver disease or disorder). In an embodiment, a level of L-valine is decreased in muscle tissue of the subject prior to treatment with a composition described herein. In an embodiment, a level of L-valine is associated with mortality in the subject. In an embodiment, L-leucine is oxidized for ammonia detoxification (e.g., muscle ammonia) in the subject.

In some embodiments, a level (e.g., in a plasma sample) of one, two, or more (e.g., all) of L-histidine, L-lysine, or L-threonine is decreased in the subject, e.g., prior to treatment with a composition described herein (e.g., relative to a healthy subject without a liver disease or disorder). In some embodiments, a decreased level of one, two, or more (e.g., all) of L-histidine, L-lysine, or L-threonine results in a decrease in protein synthesis (e.g., one or both of liver and muscle protein) in the subject.

In some embodiments, a level (e.g., in a plasma sample) of one, two, three, or more (e.g., all) of tyrosine, phenylalanine, tryptophan, or glutamine is increased in the subject, e.g., prior to treatment with a composition described herein (e.g., relative to a healthy subject without a liver disease or disorder). In some embodiments, the level of one or both of tyrosine and phenylalanine is indicative of mortality in the subject. In some embodiments, the level of glutamine is increased as a result of one or both of muscle ammonia detoxification and ammoniagenesis in the subject.

In some embodiments, the subject has a physical symptom chosen from one, two, three, four, five, six, seven, eight, or more (e.g., all) of muscle atrophy, reduced myofiber area, decreased respiratory exchange, energy deficits, decreased skeletal muscle mass, decreased quality of life, increased frequency of hospitalization, decreased success of liver transplantation, or decreased survival. In some embodiments, the method further includes monitoring the subject for an improvement in the physical symptom.

In some embodiments, a functional measure is decreased in the subject (e.g., relative to a healthy subject without a liver disease or disorder). In some embodiments, one, two, three, or more (e.g., all) of a grip strength assessment measure, chair stand assessment measure, or balance assessment measure is decreased in the subject. In some embodiments, the subject has an increased Childs-Pugh score (e.g., relative to a healthy subject without a liver disease or disorder). In some embodiments, the method further includes monitoring the subject for an improvement in one or both of the functional measure or the Childs-Pugh score.

In some embodiments, the method further includes monitoring the subject for an improvement in a symptom selected from one, two, three, four, five, six, seven, eight, nine, 10, 11, 12, 13, 14, 15, 16, 17, 18, or more (e.g., all) of hyperammonemia, ascites or complications associated with ascites, variceal bleeding, infection, hepatic encephalopathy, ammonia toxicity, hepatic insufficiency, decreased urea synthesis, inflammation of hepatic tissue, fibrosis, cirrhosis, muscle wasting, muscle catabolism, muscle atrophy, hypoalbuminemia, hypercatabolism, malnutrition, frailty, or coagulopathy.

### Improvement in Subjects

In some embodiments, the subject exhibits a restored plasma amino acid profile (e.g., an increased level of BCAAs and a decreased level of AAAs) after administration of the composition. In some embodiments, the composition is capable of increasing the Fischer’s ratio (FR) (e.g., the ratio of a level of BCAAs to a level of AAAs) in a subject, e.g., a human subject with mild to moderate hepatic insufficiency.

In some embodiments, the composition is capable of increasing the valine to phenylalanine ratio (VPR) in a subject, e.g., a human subject with mild to moderate hepatic insufficiency.

Administration of the composition can result in an improvement in body composition of a subject, e.g., the body composition of the subject is changed to a more lean phenotype (e.g., relative to a control subject). In some embodiments, the composition is capable of increasing the lean mass in a subject, e.g., a human subject with mild to moderate hepatic insufficiency.

Administration of the composition can result in an improvement in a Liver Frailty Index (LFI) of a subject. In some embodiments, the composition is capable of decreasing the LFI of a subject, e.g., a human subject with mild to moderate hepatic insufficiency.

Administration of the composition can result in an improvement (e.g., an increase) in an isoleucine concentration of a subject (e.g., a subject with cirrhosis). In some embodiments, the composition is capable of increasing the isoleucine concentration (e.g., in a plasma sample) of a subject, e.g., a subject with cirrhosis.

Administration of the composition can result in an improvement (e.g., an increase) in a leucine concentration of a subject (e.g., a subject with cirrhosis). In some embodiments, the composition is capable of increasing the leucine concentration (e.g., in a plasma sample) of a subject, e.g., a subject with cirrhosis.

Administration of the composition can result in an improvement (e.g., an increase) in a valine concentration of a subject (e.g., a subject with cirrhosis). In some embodiments, the composition is capable of increasing the valine concentration (e.g., in a plasma sample) of a subject, e.g., a subject with cirrhosis.

Administration of the composition can result in improved amino acid metabolism in a subject, e.g., a subject with cirrhosis. In some embodiments, administration of the composition comprising one or both of an ornithine amino acid entity or an aspartate amino acid entity results in improved (e.g., maintained) concentration of one, two, or three of a leucine amino acid entity, an isoleucine amino acid entity, or a valine amino acid entity in a subject, e.g., a subject with cirrhosis, e.g., in a bile duct ligation model, as described in Example 2.

Administration of the composition can result in a decreased level of tyrosine in a subject, e.g., a subject with cirrhosis. In some embodiments, an increased level of tyrosine (e.g., relative to a healthy subject without cirrhosis) is indicative of one or both of disease severity or mortality in the subject. In some embodiments, administration of the composition e.g., for a time period of 20 days, results in a decreased level of tyrosine in a subject, e.g., a subject with cirrhosis, e.g., in a bile duct ligation model, e.g., as a result of increased protein synthesis, as described in Example 2.

Administration of the composition can result in a increased Fischer’s ratio (e.g., the ratio of leucine, valine, and isoleucine to tyrosine and phenylalanine) in a subject, e.g., a subject with cirrhosis. In some embodiments, an increased level of one or both of tyrosine or phenylalanine (e.g., relative to a healthy subject without cirrhosis) is indicative of mortality in the subject.

Administration of the composition e.g., for a time period of 20 days, can result in an improved level (e.g., a decreased or maintained level) of one or both of aspartate or glutamate in a subject, e.g., a subject with cirrhosis. In some embodiments, an increased level of one or both of aspartate or glutamate (e.g., relative to a healthy subject without cirrhosis) is indicative of one or both of decreased amino acid metabolism or decreased amino acid homeostasis in the subject. In some embodiments, administration of the composition e.g., for a time period of 20 days, results in a maintained level of aspartate in a subject, e.g., a subject with cirrhosis, e.g., in a bile duct ligation model, e.g., as described in Example 2. In some embodiments, administration of the composition e.g., for a time period of 20 days, results in a decreased level of glutamate in a subject, e.g., a subject with cirrhosis, e.g., in a bile duct ligation model, e.g., as described in Example 2.

In some embodiments, administration of a composition including BCAAs (e.g., one, two, or more (e.g., all) of leucine, valine, or isoleucine) to a subject results in one, two, or more (e.g., all) of stimulated protein synthesis, detoxification of ammonia (e.g., in muscle tissue), or a restored Fischer [symbol] ratio in the subject. In some embodiments, administration of a composition including EAAs (e.g., one, two, or more (e.g., all) of histidine, lysine, and threonine) to a subject results in an increase in protein synthesis (e.g., in one or both of muscle or liver tissue) in the subject. In some embodiments, administration of a composition including UCAAs (e.g., one or two of ornithine and aspartate) to a subject results in one or both of decreased ammonia or a stimulated Urea cycle in the subject.

In some embodiments, administration of the composition results in an improvement in a symptom chosen from one, two, three, four, five, six, seven, eight, nine, 10, 11, 12, 13, 14, 15, 16, 17, 18, or more (e.g., all) of hyperammonemia, ascites or complications associated with ascites, variceal bleeding, infection, hepatic encephalopathy, ammonia toxicity, hepatic insufficiency, decreased urea synthesis, inflammation of hepatic tissue, fibrosis, cirrhosis, muscle wasting, muscle catabolism, muscle atrophy, hypoalbuminemia, hypercatabolism, malnutrition, frailty, or coagulopathy in a subject.

In some embodiments, administration of the composition promotes one or both of muscle-dependent ammonia detoxification or protein synthesis to result in one or both of decreased ammonia levels and increased muscle mass in the subject.

In some embodiments, administration of the composition results in the subject exhibiting one, two, three, or more (e.g., all) of decreased ammonia levels (e.g., hyperammonemia), increased levels of branched chain amino acids (BCAAs), decreased levels of aromatic AAs (AAAs), decreased hypercatabolism, or decreased autophagy (e.g., relative to the subject prior to administration of the composition).

In some embodiments, administration of the composition results in the subject exhibiting one, two, three, four, five, six, seven, eight, or more (e.g., all) of decreased muscle atrophy, increased myofiber area, increased respiratory exchange, increased energy, increased skeletal muscle mass, increased quality of life, decreased frequency of hospitalization, increased success of liver transplantation, or increased survival (e.g., relative to the subject prior to administration of the composition).

In some embodiments, administration of the composition results in an improvement in one or both of body weight or body composition of the subject, e.g., the body composition of the subject is changed to a more lean phenotype (e.g., relative to the subject prior to administration of the composition). In some embodiments, administration of the composition results in the subject exhibiting an increase in one, two, three, or more (e.g., all) of a grip strength assessment measure, chair stand assessment measure, or balance assessment measure (e.g., relative to the subject prior to administration of the composition). In some embodiments, administration of the composition results in the subject exhibiting an decrease in a Childs-Pugh score (e.g., relative to the subject prior to administration of the composition).

In some embodiments, administration of the composition results in the subject exhibiting one, two, three, four, five, six, seven, or more (e.g., all) of decreased ammonia levels (e.g., hyperammonemia), increased levels of BCAAs, decreased levels of AAAs, decreased catabolism, increased protein synthesis (e.g., an increased FSR, e.g., in one or both of muscle or liver tissue), decreased ROS, decreased catabolism, increased anabolism, or decreased autophagy (e.g., relative to a healthy subject without a liver disease or disorder).

### Dosage Regimens

The composition (e.g., the Active Moiety) can be administered according to a dosage regimen described herein to improve one, two, three, or more (e.g., all) of liver function, hyperammonemia, muscle mass, or muscle function in a subject, e.g., a subject with one or both of a liver disease or disorder or muscle wasting. In some embodiments, EAAs (e.g., one, two, or three of a histidine, histidine amino acid entity, and threonine) are included in the composition at a dose to achieve stoichiometry with the level of AAAs (e.g., one or both of tyrosine and phenylalanine) in a subject. In some embodiments, the dosing of the composition (e.g., in grams per day) results in one or both of the incorporation of free amino acids into muscle protein or increased anabolism in a subject.

The composition can be administered to a subject for a treatment period of, e.g., two weeks, three weeks, four weeks, five weeks, six weeks, seven weeks, eight weeks, nine weeks, 10 weeks, 11 weeks, 12 weeks, 13 weeks, 14 weeks, 15 weeks, 16 weeks, or longer at a dose of 5 g+/- 20% g daily to 100 g+/- 20% g daily, e.g., 10 g+/- 20% g daily to 75 g+/- 20% g daily. In some embodiments, the composition is administered at a dosage of 10 g+/- 20% g daily, 15 g+/-20% g daily, 20 g+/- 20% g daily, 25 +/- 20% g daily, 30 +/- 20% g daily, 35 +/- 20% g daily, 40 +/- 20% g daily, 41 +/- 20% g daily, 42 +/- 20% g daily, 43 +/- 20% g daily, 44 +/- 20% g daily, 45 +/- 20% g daily, 46 +/- 20% g daily, 47 +/- 20% g daily, 48 +/- 20% g daily, 49 +/- 20% g daily, 50 +/- 20% g daily, 55 +/- 20% g daily, or 60 +/- 20% g daily. In certain embodiments, the composition is administered at a dosage of 44 +/- 20% g daily, 45 +/- 20% g daily, 45.5 +/-20% g daily, 46 +/- 20% g daily, 46.5 +/- 20% g daily, or 48.5 +/- 20% g daily.

In some embodiments, the composition is administered with a meal. In some embodiments, the composition is administered between meals, e.g., before or after a meal. In some embodiments, the composition is administered at least once during the day and at least once in the late evening or before bedtime.

In some embodiments, the composition can be provided to a subject (e.g., a subject with a liver disease or disorder with one or both of hyperammonemia or muscle wasting), in either a single or multiple dosage regimens. In some embodiments, doses can be administered, e.g., twice daily, three times daily, four times daily, five times daily, six times daily, seven times daily, or more. In some embodiments, the composition can be administered chronically, e.g., more than 30 days, e.g., 31 days, 40 days, 50 days, 60 days, 3 months, 6 months, 9 months, one year, two years, or three years).

In some embodiments, the composition is administered every hour, every 2 hours, every 3 hours, every 4 hours, every 5 hours, every 6 hours, every 7 hours, every 8 hours, every 9 hours, every 10 hours, every 11 hours, every 11 hours, every 12 hours, every 13 hours, every 14 hours, or every 16 hours while the patient is awake. In an embodiment, one dose of the composition is administered in the late evening.

In some embodiments, the composition comprises three stick packs, e.g., each stick pack comprising 33.3% +/- 20% of the quantity of each amino acid entity included in the composition described herein. In certain embodiments, three stick packs are administered three times daily.

In some embodiments, the composition is administered at a dose of 2 g +/- 20% to 60 g +/- 20% total amino acid entities, e.g., once daily, twice daily, three times daily, four times daily, five times daily, or six times daily (e.g., three times daily). In some embodiments, the composition is administered at a dose of 2 g +/- 20% to 10 g +/- 20%, 10 g +/- 20% to 40 g +/-20%, or 40 g +/- 20% to 60 g +/- 20% total amino acid entities, e.g., once daily, twice daily, or three times daily (e.g., three times daily). In certain embodiments, the composition is administered at a dose of 10 g +/- 20% to 40 g +/- 20% total amino acid entities twice daily, e.g., 10 g +/- 20%, 15 g +/- 20%, 20 g +/- 20%, 25 g +/- 20%, 30 g +/- 20%, 35 g +/- 20%, or 40 g +/-20% total amino acid entities three times daily (e.g., 15 g +/- 20%).

In some embodiments, the composition is administered to a subject with a carbohydrate supplement, e.g., when administered in the night, late evening, or before bedtime (Table 5). In some embodiments, the composition is administered prior to, simultaneously, or subsequent to the carbohydrate supplement. In some embodiments, the composition, when administered in the late evening or before bedtime, further includes at least 50 kcal, at least 100 kcal, or at least 200 kcal of carbohydrate supplement for nocturnal dosing. In some embodiments, the carbohydrate supplement is administered at a dose of 30 g +/- 20% to 90 g +/- 20% (e.g. 55 g +/- 20%) in the late evening with the composition. In some embodiments, the carbohydrate supplement includes a polysaccharide (e.g., maltodextrin (e.g., 50 +/- 20% g of maltodextrin)) and a fermentable fiber or prebiotic (e.g., one or both of beta-glucan (e.g., 2.5 +/- 20% g of beta-glucan) or resistant starch (e.g., 2.5 +/- 20% g of resistance starch)). In some embodiments, the carbohydrate supplement is provided in a powder or liquid form and mixed with the composition for administration (e.g., at night) to a subject.

In some embodiments, administration of the composition with the carbohydrate supplement supports caloric load for overnight anabolic metabolism in a subject. In some embodiments, administration of the composition with the carbohydrate supplement modulates intestinal microbiota, e.g., to minimize nitrogen metabolism and ammonia production. In some embodiments, administration of the composition with additional constituents directs more nitrogen into protein and/or less nitrogen into waste. In some embodiments, administration of the composition with fibers (e.g. nonfermentable fibers), starches (e.g., resistant starches), and/or glycans (e.g., specific or mixtures of glycans), e.g., to minimize fecal nitrogen deposition, increases the efficacy of the composition.

TABLE 5

Exemplary carbohydrate supplement for administration with the composition

| Component | Grams | Est kcal/g | Est kcal | Exemplary Function in Composition |
|---|---|---|---|---|
| Maltodextrin | 50 | 4 | 200 | Polyaccharide as a major source of glucose and caloric content. |
| Beta-glucan | 2.5 | 4 | 10 | Fermentable fiber/prebiotic to slow absorption of glucose (from maltodextrin), provide additional caloric content, and promote restorative changes in gut microbiota. |
| Resistant Starch | 2.5 | 2 | 5 | Fermentable fiber/prebiotic to slow absorption of glucose (from maltodextrin), provide additional caloric content, and promote restorative changes in gut microbiota. |
| Total carbs/day | 55 | | | |
| Estimated cal/day | | | 215 | |

### Formulations and Kits

Formulations, e.g., dosage formulations, and kits, e.g., therapeutic kits, that include the composition comprising amino acid entities and/or one or more therapeutic agents, and instructions for use, are also disclosed. The composition comprising amino acid entities and the one or more therapeutic agents can be administered via the same administration route or via different administration routes.

The combinations and/or compositions of this disclosure may be in a variety of forms. These include, for example, liquid, semi-solid, and solid dosage forms, such as liquid solutions (e.g., injectable and infusible solutions), dispersions or suspensions, liposomes, and suppositories. In some embodiments, the combination and/or composition (e.g., one or more therapeutic agents) is in the form of injectable or infusible solutions. In other embodiments, administration is parenteral (e.g., intravenous, subcutaneous, intraperitoneal, or intramuscular). In certain embodiments, administration is by an intravenous infusion or injection. In certain embodiments, administration is by intramuscular or subcutaneous injection.

The combination (e.g., the composition comprising amino acid entities (e.g., an Active Moiety) and/or one or more therapeutic agents) of the present disclosure may be formulated in a form suitable for oral use. For example, the combination and/or composition may be formulated in dry form as a powder, e.g., in a sachet, vial, stick pack, or dispersible powder or granules. In other embodiments, the combination and/or composition is formulated in liquid form, e.g., as an aqueous or oily suspension, emulsion, syrup, gel pack, or elixir. In some embodiments, the combination and/or composition formulated in dry form can be dissolved in an appropriate solvent to provide the combination and/or composition formulated in liquid form. The combination and/or composition may be accompanied by instructions for adding the dry combination to liquid. The combination and/or composition of the present disclosure may be formulated in a form suitable for enteral administration (e.g., via tube feeding).

The one or more therapeutic agents can be formulated as a solution, microemulsion, dispersion, liposome, or other ordered structure. Sterile injectable solutions can be prepared by incorporating the active compound (e.g., the one or more therapeutic agents) in the required amount in an appropriate solvent with one or a combination of ingredients enumerated herein, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, e.g., by the use of a coating such as lecithin, by the maintenance of the required particle size in the case of dispersion and by the use of surfactants. Prolonged absorption of injectable compositions can be brought about by including an agent that delays absorption, e.g., monostearate salts and/or gelatin.

In some embodiments, the combination and/or composition meets a standard for sterility, e.g., a reference standard for sterility. In some embodiments, the standard for sterility is more sterile than the standard of sterility for food. In some embodiments, the standard for sterility is less sterile than the standard of sterility required for parenteral administration.

Also within the scope of the invention is a kit comprising a combination and/or composition described herein. The kit can include one or more other elements including, but not limited to: instructions for use; other reagents (e.g., a label, an agent useful for chelating, or otherwise coupling, a therapeutic agent to a label); devices or other materials for preparing the combination for administration; pharmaceutically acceptable carriers; and devices or other materials for administration to a subject. In some embodiments, the composition comprising amino acid entities (e.g., an Active Moiety) and the one or more therapeutic agents are supplied in the same kit (e.g., as a separated dosage form in the same packaging). In other embodiments, the composition comprising amino acid entities and the one or more therapeutic agents are supplied in separate kits.

### Excipients

The combination and/or composition of the present disclosure may be compounded or formulated with one or more excipients. Non-limiting examples of suitable excipients include a tastant, a flavorant, a buffering agent, a preservative, a stabilizer, a binder, a compaction agent, a lubricant, a dispersion enhancer, a disintegration agent, a flavoring agent, a sweetener, and a coloring agent.

In some embodiments, the excipient comprises a buffering agent. Non-limiting examples of suitable buffering agents include citric acid, sodium citrate, magnesium carbonate, magnesium bicarbonate, calcium carbonate, and calcium bicarbonate. In some embodiments, the excipient comprises a preservative. Non-limiting examples of suitable preservatives include antioxidants, such as α-tocopherol and ascorbate, and antimicrobials, such as parabens, chlorobutanol, and phenol.

In some embodiments, the combination and/or composition comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, and combinations thereof.

In some embodiments, the combination and/or composition comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

In some embodiments, the combination and/or composition comprises a dispersion enhancer as an excipient. Non-limiting examples of suitable dispersants include starch, alginic acid, polyvinylpyrrolidones, guar gum, kaolin, xanthan gum, bentonite, purified wood cellulose, sodium starch glycolate, isoamorphous silicate, and microcrystalline cellulose as high HLB emulsifier surfactants.

In some embodiments, the combination and/or composition comprises a disintegrant as an excipient. In some embodiments, the disintegrant is a non-effervescent disintegrant. Non-limiting examples of suitable non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth. In some embodiments, the disintegrant is an effervescent disintegrant. Non-limiting examples of suitable effervescent disintegrants include sodium bicarbonate in combination with citric acid, and sodium bicarbonate in combination with tartaric acid.

In some embodiments, the excipient comprises a flavoring agent. Flavoring agents can be chosen from synthetic flavor oils and flavoring aromatics; natural oils; extracts from plants, leaves, flowers, and fruits; and combinations thereof. In some embodiments, the flavoring agent is selected from cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

In some embodiments, the excipient comprises a sweetener. Non-limiting examples of suitable sweeteners include glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, xylitol, and the like. Also contemplated are hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

In some embodiments, the combination and/or composition comprises a coloring agent. Non-limiting examples of suitable color agents include food, drug and cosmetic colors (FD&C), drug and cosmetic colors (D&C), and external drug and cosmetic colors (Ext. D&C). The coloring agents can be used as dyes or their corresponding lakes.

In some embodiments, the combination and/or composition comprises a bitterness modifying agent. The bitterness modifying agent may reduce the bitterness or the perceived bitterness of the combination. In some embodiments, the bitterness modifying agent is a bitterness covering agent, e.g., that increases a non-bitter flavor so that the taste perceived is less bitter. Bitterness covering agents include sweeteners. Examples of bitterness modifying agents, e.g., bitterness covering agents, include fenchone, borneol or isoborneol, menthol, and aspartame.

Particular excipients may include one or more of: citric acid, lecithin, (e.g., Alcolec F 100), sweeteners (e.g. sucralose, sucralose micronized NF, acesulfame potassium (e.g., Ace-K)), a dispersion enhancer (e.g., xanthan gum (e.g. Ticaxan Rapid-3)), flavorings (e.g., vanilla custard #4306, Nat Orange WONF #1326, lime 865.0032U, and lemon 862.2169U), a bitterness masking agent (e.g., 936.2160U), and natural or artificial colorings (e.g., FD&C Yellow 6). Exemplary ingredient contents for each stick pack are shown in Table 6.

TABLE 6

Exemplary contents in each stick pack

| | | |
|---|---|---|
| INGREDIENT | GRADE | FUNCTION |
| Amino Acids | USP | Active Pharmaceutical Ingredient (API) |
| Citric Acid | USP | pH, Flavor |
| Acesulfarne K | NF | Sweetness (rapid onset) |
| Sucralose | NF | Sweetness (slow onset) |
| Lecithin (Alecolec F100) | FCC | Wetting Agent |
| Xanthan Gum | FCC | Stabilizer/Thickener |
| Vanilla Custard (Art) | GRAS | Taste/Aroma |
| Orange (Natural and WONF) | GRAS | 1° flavor |
| Lime (Natural and WONF) | GRAS | 2° flavor |
| Lemon (Natural and artificial) | GRAS | 2° flavor |
| Taste Modifier | GRAS | Bitterness masking |
| FD&C Yellow No. 6 | USP | Color |

In another embodiment, excipients are limited to citric acid, a sweetener (e.g., sucralose), xanthan gum, an aroma agent (e.g., vanilla custard #4036), a flavoring agent (e.g., Nat orange WONF #1362), and a coloring agent (e.g., FD&C Yellow 6), e.g., the excipient specifically excludes lecithin (Table 7).

TABLE 7

Exemplary contents in each stick pack

| | | |
|---|---|---|
| INGREDIENT | GRADE | FUNCTION |
| Amino Acids | USP | Active Pharmaceutical Ingredient (API) |
| Citric Acid | USP | pH, Flavor |
| Sucralose | NF | Sweetness (slow onset) |
| Xanthan Gum | FCC | Stabilizer/Thickener |
| Vanilla Custard (Art) | GRAS | Aroma |
| Orange (Nat+ WONF) | GRAS | 1° flavor |
| FD&C Yellow No. 6 | USP | Color |

### Excipient Formulation Organoleptic Properties

In some embodiments, methods described herein further comprise evaluating an oral administration quality of the combination and/or composition. Oral administration qualities include but are not limited to flavor, taste, aroma, texture, mouth feel, and color. As used herein, flavor as a quality comprises taste and aroma. In some embodiments, flavor as a quality further comprises texture and/or mouth feel. Flavor is a composite characteristic that describes the sensations associated with a substance in a subject’s mouth. In some embodiments, methods described herein further comprise evaluating a flavor of the combination and/or composition. In some embodiments, the general flavor of the combination and/or composition is evaluated. In some embodiments, particular aspects of flavor are evaluated (e.g., bitterness, sweetness, sourness, saltiness, umami-ness, savoriness, metallic notes, or chemical burn). As used herein, taste as a quality comprises the sensations associated with a substance as experienced purely by the tongue/oral cavity, e.g., in the absence of olfactory/retronasal olfaction contribution. As used herein, aroma as a quality comprises the sensations associated with a substance as experienced via olfaction, e.g., including retronasal olfaction.

As used herein, texture as a quality comprises a property of a substance (e.g., a combination and/or composition) that describes its granularity and consistency. For example, a substance may have a texture that is crumbly, tough, viscous, clumpy, etc. In some embodiments, texture is typically applied to solids, e.g., powders or foods. As used herein, mouthfeel as a quality comprises the touch sensations associated with a substance as experienced by the mouth.

In some embodiments, the combination and/or composition is evaluated in dry form, e.g., as a powder. In some embodiments, the combination and/or composition is evaluated in liquid form, e.g., as an aqueous or oily suspension, emulsion, syrup, gel pack, or elixir. In some embodiments, the combination and/or composition is evaluated in the form of a dietary composition, e.g., chosen from a medical food, a functional food, a supplement, or a nutraceutical.

Many methods of evaluating oral administration qualities are known to those skilled in the art and may be used to evaluate oral administration qualities in the methods described herein.

In some embodiments, evaluating an oral administration quality comprises evaluating the flavor, e.g., taste and/or aroma, of a combination. In some embodiments, evaluating flavor comprises evaluating one, two, three, four, or all of the level of bitterness, sweetness, sourness, saltiness, umami-ness, savoriness, metallic notes, or chemical burn of a combination. Bitterness can be evaluated by an animal preference test, human sensory evaluation (e.g., a group of human tasters evaluating a number of substances, e.g., scoring them relative to the combination), by electronic tongue measurement, by in silico predictive methods (e.g., using software or searchable databases that, based on the structural/chemical features of the components of the combination, predicts the bitterness of the combination), or other methods known in the art.

Excipients, e.g., oral administration components, can modify the oral administration qualities of the combination. In some embodiments, the methods described herein further comprise, responsive to an evaluation of an oral administration quality, the addition or further addition of an excipient, e.g., oral administration component. In some embodiments, the excipient, e.g., oral administration component, added or further added masks or lessens the bitterness of the combination.

### Production of Combinations and/or Compositions

The present disclosure features a method of manufacturing or making a composition (e.g., an Active Moiety) of the foregoing invention. Amino acid entities used to make a combination and/or composition of the invention may be agglomerated and/or instantized to aid in dispersal and/or solubilization. The combinations and/or compositions may be made using amino acid entities from the following sources, or other sources may used: e.g., FUSI-BCAA™ Instantized Blend (L-Leucine, L-Isoleucine and L-Valine in 2:1:1 weight ratio), instantized L-Leucine, and other acids may be obtained from Ajinomoto Co., Inc. Pharma. grade amino acid entity raw materials may be used in the manufacture of pharmaceutical amino acid entity products. Food (or supplement) grade amino acid entity raw materials may be used in the manufacture of dietary amino acid entity products.

To produce the compositions comprising amino acid entities (e.g., for the combinations of the instant disclosure), the following general steps may be used: the starting materials (individual amino acid entities and excipients) may be blended in a blending unit, followed by verification of blend uniformity and amino acid entity content, and filling of the blended powder into stick packs or other unit dosage form. The content of stick packs or other unit dosage forms may be dispersed in water at time of use for oral administration. Food supplement and medical nutrition combinations of the invention may be in a form suitable for oral administration.

When combining raw materials, e.g., pharmaceutical grade amino acid entities and/or excipients, into a combination and/or composition described herein, contaminants may be present in the combination and/or composition. A combination and/or composition meets a standard for level of contamination when the combination and/or composition does not substantially comprise (e.g., comprises less than 10, 9, 8, 7, 6, 5, 4, 3, 2, 1, 0.1, 0.01, or 0.001% (w/w)) a contaminant. In some embodiments, a combination and/or composition described in a method herein does not comprise a contaminant. Contaminants include any substance that is not deliberately present in the combination and/or composition (for example, pharmaceutical grade amino acid entities and excipients, e.g., oral administration components, may be deliberately present) or any substance that has a negative effect on a product quality parameter of the combination (e.g., side effects in a subject, decreased potency, decreased stability/shelf life, discoloration, odor, bad taste, bad texture/mouthfeel, or increased segregation of components of the combination). In some embodiments, contaminants include microbes, endotoxins, metals, or a combination and/or composition thereof. In some embodiments, the level of contamination, e.g., by metals, lecithin, choline, endotoxin, microbes, or other contaminants (e.g., contaminants from raw materials) of each portion of a combination and/or composition is below the level permitted in food.

### Dietary Combinations and/or Compositions

The combinations and/or composition disclosed herein can comprise dietary compositions, e.g., dietary compositions comprising amino acid entities as described herein. The composition comprising amino acid entities (e.g., Active Moiety) can be formulated and used as a dietary composition, e.g., chosen from a medical food, a functional food, or a supplement. In such an embodiment, the raw materials and final product should meet the standards of a food product. In some embodiments, the dietary composition is for use in a method, comprising administering the composition comprising amino acid entities (e.g., Active Moiety) in combination with a therapeutic agent described herein to a subject. The composition can be for use in a dietary composition for the purpose of improving liver function or a liver disease or disorder, e.g., in combination with a therapeutic agent described herein.

In some embodiments, the composition comprising amino acid entities (e.g., an Active Moiety) is in the form of a nutritional supplement, a dietary formulation, a functional food, a medical food, a food, or a beverage comprising a composition described herein. In some embodiments, the nutritional supplement, the dietary formulation, the functional food, the medical food, the food, or the beverage comprising a composition comprising amino acid entities described herein for use in the management of a liver disease or disorder (e.g., in a subject with a liver disease or disorder).

The present disclosure features a method of improving a liver disease or disorder comprising administering to a subject an effective amount of a composition comprising amino acid entities described herein (e.g., an Active Moiety), e.g., in combination with a therapeutic agent described herein. The present disclosure features a method of providing nutritional support or supplementation to a subject with a liver disease or disorder (e.g., a subject with a liver disease or disorder), comprising administering to the subject an effective amount of a composition comprising amino acid entities described herein.

The present disclosure features a method of providing nutritional support or supplementation that aids in the management of a liver disease or disorder (e.g., a liver disease or disorder), comprising administering to a subject in need thereof an effective amount of a composition comprising amino acid entities described herein (e.g., an Active Moiety). In some embodiments, the subject has or has been diagnosed with a liver disease or disorder. In other embodiments, the subject does not have a liver disease or disorder. The composition comprising amino acid entities (e.g., an Active Moiety) can be used in methods of dietary management of a subject (e.g., a subject without a liver disease or disorder).

In some embodiments, the subject has a fatty liver disease or disorder. In some embodiments, the fatty liver disease or disorder is chosen from: non-alcoholic fatty liver disease (NAFLD) or alcoholic fatty liver disease (AFLD). In certain embodiments, the NAFLD is chosen from NASH or non-alcoholic fatty liver NAFL. In certain embodiments, the subject has pediatric NAFLD. In certain embodiments, the AFLD is ASH. In some embodiments, the subject has one or both of fibrosis and steatosis. In certain embodiments, the subject (e.g., a subject with NASH) has cirrhosis. In some embodiments, the subject has hepatocarcinoma. In certain embodiments, the subject has one or both of an increased risk of liver failure or an increased risk of death.

In some embodiments, the subject has one, two, three, or more (e.g., all) of diabetes (e.g., type 2 diabetes), metabolic syndrome, a relatively high BMI, or obesity. In some embodiments, the composition comprising amino acid entities promotes weight loss in the subject. In some embodiments, the subject has one, two, or more (e.g., all) of gut leakiness, gut dysbiosis, or gut microbiome disturbance. In some embodiments, the subject has diabetic peripheral neuropathy.

In some embodiments, the dietary composition is for use in a method, comprising administering the composition to a subject. The composition can be for use in a dietary composition for the purpose of improving one, two, three, or more (e.g., all) of liver function, hyperammonemia, muscle mass, or muscle function in a subject.

In some embodiments, the composition is in the form of a nutritional supplement, a dietary formulation, a functional food, a medical food, a food, or a beverage comprising a composition described herein. In some embodiments, the nutritional supplement, the dietary formulation, the functional food, the medical food, the food, or the beverage comprising a composition described herein for use in the management of a liver disease or disorder with one or both of hyperammonemia or muscle wasting (e.g., cirrhosis, e.g., cirrhotic sarcopenia, End Stage Liver Disease, hepatic insufficiency, hepatic encephalopathy, or a combination thereof) in a subject.

In some embodiments, the dietary composition is administered to a subject with a carbohydrate supplement, e.g., when administered in the night, late evening, or before bedtime. In some embodiments, the dietary composition is administered prior to, simultaneously, or subsequent to the carbohydrate supplement. In some embodiments, the dietary composition, when administered in the late evening or before bedtime, further includes at least 50 kcal, at least 100 kcal, or at least 200 kcal of carbohydrate supplement for nocturnal dosing. In some embodiments, the carbohydrate supplement is administered at a dose of 30 g +/- 20% to 90 g +/-20% (e.g. 55 g +/- 20%) in the late evening with the dietary composition. In some embodiments, the carbohydrate supplement includes a polysaccharide (e.g., maltodextrin (e.g., 50 +/- 20% g of maltodextrin)) and a fermentable fiber or prebiotic (e.g., one or both of beta-glucan (e.g., 2.5 +/-20% g of beta-glucan) or resistant starch (e.g., 2.5 +/- 20% g of resistance starch)). In some embodiments, the carbohydrate supplement is provided in a powder or liquid form and mixed with the dietary composition for administration (e.g., at night) to a subject.

In some embodiments, administration of the dietary composition with the carbohydrate supplement supports caloric load for overnight anabolic metabolism in a subject. In some embodiments, administration of the dietary composition with the carbohydrate supplement modulates intestinal microbiota, e.g., to minimize nitrogen metabolism and ammonia production. In some embodiments, administration of the dietary composition with additional constituents directs more nitrogen into protein and/or less nitrogen into waste. In some embodiments, administration of the dietary composition with fibers (e.g. nonfermentable fibers), starches (e.g., resistant starches), and/or glycans (e.g., specific or mixtures of glycans), e.g., to minimize fecal nitrogen deposition, increases the efficacy of the dietary composition.

The present disclosure features a method of of improving one, two, three, four, five, six, seven, eight, nine, 10, 11, 12, 13, 14, 15, 16, 17, 18, or more (e.g., all) of hyperammonemia, ascites or complications associated with ascites, variceal bleeding, infection, hepatic encephalopathy, ammonia toxicity, hepatic insufficiency, decreased urea synthesis, inflammation of hepatic tissue, fibrosis, cirrhosis, muscle wasting, muscle catabolism, muscle atrophy, hypoalbuminemia, hypercatabolism, malnutrition, frailty, or coagulopathy, comprising administering to a subject an effective amount of a dietary composition described herein.

The present disclosure features a method of providing nutritional support or supplementation to a subject with a liver disease or disorder with one or both of hyperammonemia or muscle wasting, comprising administering to the subject an effective amount of a composition described herein. In some embodiments, the subject has cirrhosis. In some embodiments, the subject has cirrhotic sarcopenia. In some embodiments, the subject has hepatic insufficiency. In some embodiments, the subject has End Stage Liver Disease. In some embodiments, the subject has hepatic encephalopathy.

The present disclosure features a method of providing nutritional support or supplementation that aids in the management of a liver disease or disorder with one or both of hyperammonemia or muscle wasting, comprising administering to a subject in need thereof an effective amount of a composition described herein.

The compositions can be used in methods of dietary management of a subject (e.g., a subject without a liver disease or disorder with one or both of hyperammonemia or muscle wasting). In some embodiments, the subject does not have a liver disease or disorder with one or both of hyperammonemia or muscle wasting.

### Biomarkers

Any of the methods disclosed herein can include evaluating or monitoring the effectiveness of administering a combination of the invention as described herein to a subject with a liver disease or disorder (e.g., NAFLD (e.g., NASH or NAFL) or AFLD (e.g., ASH)). In some embodiments, the value of effectiveness of the combination in treating a subject with a liver disease (e.g., NAFLD (e.g., NASH or NAFL) or AFLD (e.g., ASH)) comprises a measure of one, two, three, four, five, six, seven, eight, nine, 10, 11, 12, 13, 14, 15,16, 17, 18, 19, 20, 21, 22, or more (e.g., all) of: a) alanine aminotransferase (ALT); b) aspartate aminotransferase (AST); c) adiponectin; d) N-terminal fragment of type III collagen (proC3); e) caspase-cleaved keratin 18 fragments (M30 and M65); f) IL-1β; g) C-reactive protein; h) PIIINP; i) a tissue inhibitor of metalloproteinase (TIMP); e.g., TIMP1 or TIMP2; j) MCP-1; k) FGF-21; 1) Colla1; m) Acta2; n) a matrix metalloproteinase (MMP), e.g., MMP-13, MMP-2, MMP-9, MT1-MMP, MMP-3, or MMP-10; o) ACOX1; p) IL-10; q) NF-kB; r) TNF-α; s) hydroxyproline; t) IL-2; u) MIP-1; v) α-SMA; or w) TGF-β.

In some embodiments, the subject exhibits increased levels of one, two, three, four, five, six, seven, eight, nine, 10, 11, 12, 13, 14, 15,16, 17, 18, 19, 20, or more (e.g., all) of: ALT; AST; proC3; caspase-cleaved keratin 18 fragments (M30 and M65); IL-1β; C-reactive protein; PIIINP; a TIMP (e.g., TIMP1 or TIMP2); MCP-1; FGF-21; Colla1; Acta2; a MMP (e.g., MMP-13, MMP-2, MMP-9, MT1-MMP, MMP-3, or MMP-10); ACOX1; NF-kB; TNF-α; hydroxyproline; IL-2; MIP-1; α-SMA; or TGF-β, e.g., relative to a healthy subject without a liver disease or disorder. In some embodiments, the subject exhibits one or both of decreased levels of IL-10 or adiponectin, e.g., relative to a healthy subject without a liver disease or disorder.

In some embodiments, administration of the combination (e.g., at a dosage regimen described herein) to the subject reduces the level or activity of one, two, three, four, five, six, seven, eight, nine, 10, 11, 12, 13, 14, 15,16, 17, 18, 19, 20, or more (e.g., all) of: ALT; AST; proC3; caspase-cleaved keratin 18 fragments (M30 and M65); IL-1β; C-reactive protein; PIIINP; a TIMP (e.g., TIMP1 or TIMP2); MCP-1; FGF-21; Colla1; Acta2; a MMP (e.g., MMP-13, MMP-2, MMP-9, MT1-MMP, MMP-3, or MMP-10); ACOX1; NF-kB; TNF-α; hydroxyproline; IL-2; MIP-1; α-SMA; or TGF-β. In some embodiments, administration of the combination to the subject increases the level or activity of one or both of IL-10 or adiponectin.

Any of the methods disclosed herein can include evaluating or monitoring the effectiveness of administering a composition including amino acid entities to a subject, e.g., a subject having a liver disease or disorder with one or both of hyperammonemia or muscle wasting (e.g., cirrhosis, e.g., cirrhotic sarcopenia, End Stage Liver Disease, hepatic insufficiency, hepatic encephalopathy, or a combination thereof).

In embodiments, the value of effectiveness to the composition in treating a subject comprises a measure of one, two, three, four, five, six, seven, eight, nine, 10, 11, or more (e.g., all) of the following: a) the ratio of BCAAs to AAAs (e.g., the Fischer’s ratio), b) a level of ammonia, c) a level of valine relative to a level of phenylalanine, d) a level of albumin (e.g. meal-induced albumin), e) a level of myostatin, f) a level or activity of mTOR, g) a level of creatinine, h) a level of bilirubin, i) a level of urinary 3-methylhistidine, j) a level of AMPK, k) a level of Gcn2, or 1) a level of protein synthesis.

In some embodiments of any of the methods disclosed herein, the measure of one or more of a)-1) is obtained from a sample acquired from the subject. In some embodiments, the sample is chosen from a blood sample (e.g., a plasma sample), a liver sample, or a muscle sample.

In some embodiments, the subject is evaluated prior to receiving, during, or after receiving, a composition including defined amino acid components.

In some embodiments, the level of BCAAs and AAAs are determined using a Fischer’s Ratio.

In some embodiments, administration of the composition results in one, two, three, four, five, six, seven, eight, nine, 10, 11, or more (e.g., all) of: a) increased level of BCAAs to AAAs (e.g., increased Fischer’s ratio), b) decreased level of ammonia, c) an increased level of valine relative to a level of phenylalanine, d) an increased level of albumin (e.g. meal-induced albumin), e) a decreased level of myostatin, f) an increased level of activity of mTOR, g) a decreased level of creatinine, h) a decreased level of bilirubin, i) a decreased level of urinary 3-methylhistidine, j) a decreased level of AMPK, k) a decreased level of Gcn2, or 1) an increased level of protein synthesis.

In some embodiments, administration of the composition results in an increase in amino acid entities (e.g., one, two, there, four, five, or six of L-valine, L-leucine, L-isoleucine, L-histidine, L-lysine, or L-threonine) in one, two, or more (e.g., all) of blood, plasma, or serum of the subject, e.g., in a blood, plasma, or serum sample from the subject.

In some embodiments, administration of the composition results in an improvement in one, two, three, four, five, six, seven, eight, nine, 10, 11, or more (e.g., all) of a)-1) after a treatment period of 24 hours (e.g., after 48 hours or 72 hours).

### EXAMPLES

The Examples below are set forth to aid in the understanding of the inventions, but is not intended to, and should not be construed to, limit its scope in any way.

### Example 1. Hepatocyte Model for Steatosis and Inflammation

Hepatocyte lipotoxicity appears to be a central driver of hepatic cellular injury via oxidative stress and endoplasmic reticulum (ER) stress. The ability of amino acids combined to therapeutics agents (Rx) to influence steatosis (lipid accumulation) and inflammation in hepatocytes was assessed using human primary hepatocytes purchased from two sources: Lonza and Sekisui Xenotech.

### Cell Seeding and Maintenance

Primary hepatocytes from two healthy human donor were seeded on day 0 at density of 60000 cells in 96 well optical microplates (Thermofisher) in hepatocyte plating media (William’s E medium (Gibco) supplemented with 10% heat-inactivated FBS (Atlanta Bio), 2mM Glutamax (Gibco), and 0.2% Primocin (InVivoGen) and incubated for 6 hours at 37° C., 5% CO₂. After 6 hours, cells were washed twice and incubated overnight at 37° C., 5% CO₂ in hepatocytes plating media. On day 1, cells were washed twice and incubated for 24h in Hepatocytes defined medium (Corning) supplemented with 2mM Glutamax (Gibco), and 1x Penicillin/Streptomycin in the same conditions described above.

### Amino Acids Pre-Treatment

On day 2, cells were washed twice with DPBS 1X (Gibco) and maintained in amino acid-free WEM (US Biologicals) containing a defined custom amino acid concentration based on the mean physiological concentrations in blood. The values are published in the Human Metabolome Database (Wishart DS, Tzur D, Knox C, et al., HMDB: the Human Metabolome Database. Nucleic Acids Res. 2007 Jan; 35(Database issue):D521-6. 17202168; which is hereby incorporated by reference in its entirety). This custom media is supplemented with either serially diluted therapeutic agents (Rx) (Selonsertib, Firsocostat, Cilofexor and Tropifexor) or with a defined amino acid compositions LIRQSNacCar, LIRQ[2.5]S[10]NacCar and NacCar at 20x concentration above the plasma level or with a combination of both (Rx-AA composition). For baseline control the same custom media is supplemented with similar volume of PBS1x or DMSO (0.1%). Cells were maintained in this defined pretreatment media for 24 hours at 37° C., 5% CO₂.

### Co-Treatment With Free Fatty Acids and Different Amino Acids Combination

After pre-treatment, cells were co-treated with saturated fatty acids (sFA) at 250 uM with a ratio of 2:1 (Oleate:Palmitate) and TNF-α (Thermofisher) at 1 ng/ml (sFA+TNF-α) and supplemented with the amino acids combination (LIRQSNacCar, LIRQ[2.5]S[10]NacCar, NacCar) and Rx individually or in combinations for 24 hours at 37° C., 5%CO₂. After 24 hours incubation, media was removed for chemokine analysis (MCP1) and the same co treatment was reapplied for an additional 48 hours for a total of 72 hours.

### Intracellular Lipid Accumulation Analysis After 72h by Fluorescence Microscopy

After 72 hours, cells were washed twice with PBS 1x (Gibco), fixed with 4% Paraformaldehyde, and washed twice with PBS 1x (100 ul). After fixation, lipids were stained with HCS LipidTOX Red Neutral (Thermofisher Scientific) diluted 1000x and nuclei were stained with Hoechst 3342 (Life Technologies) diluted to 4ug/ml. The LipidTOX™ neutral lipid stain has an extremely high affinity for neutral lipid droplets that was detected by fluorescence microscopy using a high content imager (Molecular Devices). Lipid was normalized to the specific per well cell density determined by nuclei count. Images were analyzed and total lipid area was calculated using MetaXpress 6 software.

### Cytokine Analysis After 24h by ELISA

Human CCL2 (MCP-1) was measured by ELISA (Human CCK2/MCP-1 DuoSet ELISA, R&D Systems) at 1/20 dilution in 1X Reagent Diluent (Reagent Ancillary Kit 2, R&D Systems). Lipid was normalized to the specific per well cell density determined by nuclei count.

### Results 1 (Donor 1 and 2)

### Lipid Accumulation and Steatosis Phenotypes

Tables E1 and E2 shows the level of total lipid area subtracted from the baseline (sFA+TNFa) and normalized to nuclei count in primary human hepatocytes cells from two healthy donors, 1 and 2 respectively. Treatment with LIRQSNacCar or LIRQ[2.5]S[10]NacCar at 20X reduced lipid level compared to baseline (Table E1 & E2, column 5). Treatment with the therapeutic agents (Rx) shows a slight reduction in lipid level when treated with Firsocostat and Selonsertib, and no changes to a slight increase in lipid level when treated with Cilofexor or Tropifexor, respectively (Table E1 & E2, column 8). In addition, the impact on lipid of the combination of each of LIRQSNacCar and LIRQ[2.5]S[10]NacCar at 20X with 4 different doses of the Rx (Selonsertib, Firsocostat, Cilofexor and Tropifexor) was measured. For each Rx-amino acids composition combination, we calculated a synergy score (Table E1 & E2, column 12) by subtracting the expected effect of the given components of the combination from the observed effect of the same combination components. The expected effect of a combination was defined as the sum of the effect of individual Rx and amino acids composition. Synergy score of 0 indicates no interaction (expected = observed). A negative or positive synergy score indicates that the observed effect is smaller or larger than the expected effect, respectively. A synergy score that is different than zero suggests an interaction between the amino acid composition and the tested Rx. As reducing lipid is the desired phenotype, a negative synergy score corresponds to a stronger reduction of lipid in the Rx-AA composition combination compared to the individual treatments. A low synergy score was observed with Firsocostat treatment in combination with either LIRQSNacCar or LIRQ[2.5]S[10]NacCar in both donors at all drug concentrations, suggesting that the combination Firsocostat-AA composition reduces lipid at a higher level than Firsocostat, LIRQSNacCar or LIRQ[2.5]S[10]NacCar alone. Selonsertib treatment in combination with either LIRQSNacCar or LIRQ[2.5]S[10]NacCar show a synergy score that is lower than 0 at different doses of the drug in both donors, suggesting that the combination Selonsertib-AA composition reduces lipid better than Selonsertib, LIRQSNacCar or LIRQ[2.5]S[10]NacCar alone. Cilofexor in combination with LIRQ[2.5]S[10]NacCar shows a synergy score that is lower than 0 at all the drug doses in donor 1 and only at 2 drug doses in donor 2. However, when Cilofexor is combined with LIRQSNacCar, it shows a synergy score lower than 0 at two doses of the drugs in both donors. Treatment with Tropifexor in combination with either LIRQSNacCar or LIRQ[2.5]S[10]NacCar composition shows a synergy score that is higher than 0 at all the drug doses tested in donor 1. However, in donor 2, treatment of Tropifexor in combination with LIRQ[2.5]S[10]NacCar shows a synergy score that is lower than 0 with 3 drug doses and with 1 drug dose when in combination with LIRQSNacCar.

TABLE E1

| Row | Donor | phenotype | Amino Acid | AA Conc. (X)* | AA+Supp. effect | Rx | Rx Conc. (µM) | Rx effect |
|---|---|---|---|---|---|---|---|---|
| | | | Supplement (Supp.) | Nac/Car (mM) | | | | |
| 1 | 1 | Lipid | LIRQ[2.5]S[10]; NacCar | 20X; 5mM | -1.425 | Selonsertib | 0.063 | -0.060 |
| 2 | 1 | Lipid | LIRQ[2.5]S[10]; NacCar | 20X; 5mM | -1.425 | Selonsertib | 0.250 | -0.045 |
| 3 | 1 | Lipid | LIRQ[2.5]S[10]; NacCar | 20X; 5mM | -1.425 | Selonsertib | 1.000 | -0.154 |
| 4 | 1 | Lipid | LIRQ[2.5]S[10]; NacCar | 20X; 5mM | -1.425 | Selonsertib | 4.000 | -0.152 |
| 5 | 1 | Lipid | LIRQS; NacCar | 20X; 5mM | -0.412 | Selonsertib | 0.063 | -0.060 |
| 6 | 1 | Lipid | LIRQS; NacCar | 20X; 5mM | -0.412 | Selonsertib | 0.250 | -0.045 |
| 7 | 1 | Lipid | LIRQS; NacCar | 20X; 5mM | -0.412 | Selonsertib | 1.000 | -0.154 |
| 8 | 1 | Lipid | LIRQS; NacCar | 20X; 5mM | -0.412 | Selonsertib | 4.000 | -0.152 |
| 9 | 1 | Lipid | LIRQ[2.5]S[10]; NacCar | 20X; 5mM | -1.425 | Firsocostat | 0.063 | -0.210 |
| 10 | 1 | Lipid | LIRQ[2.5]S[10]; NacCar | 20X; 5mM | -1.425 | Firsocostat | 0.250 | -0.284 |
| 11 | 1 | Lipid | LIRQ[2.5]S[10]; NacCar | 20X; 5mM | -1.425 | Firsocostat | 1.000 | -0.412 |
| 12 | 1 | Lipid | LIRQ[2.5]S[10]; NacCar | 20X; 5mM | -1.425 | Firsocostat | 4.000 | -0.317 |
| 13 | 1 | Lipid | LIRQS; NacCar | 20X; 5mM | -0.412 | Firsocostat | 0.063 | -0.210 |
| 14 | 1 | Lipid | LIRQS; NacCar | 20X; 5mM | -0.412 | Firsocostat | 0.250 | -0.284 |
| 15 | 1 | Lipid | LIRQS; NacCar | 20X; 5mM | -0.412 | Firsocostat | 1.000 | -0.412 |
| 16 | 1 | Lipid | LIRQS; NacCar | 20X; 5mM | -0.412 | Firsocostat | 4.000 | -0.317 |
| 17 | 1 | Lipid | LIRQ[2.5]S[10]; NacCar | 20X; 5mM | -1.314 | Cilofexor | 0.031 | 0.019 |
| 18 | 1 | Lipid | LIRQ[2.5]S[10]; NacCar | 20X; 5mM | -1.314 | Cilofexor | 0.125 | 0.072 |
| 19 | 1 | Lipid | LIRQ[2.5]S[10]; NacCar | 20X; 5mM | -1.314 | Cilofexor | 0.500 | 0.027 |
| 20 | 1 | Lipid | LIRQ[2.5]S[10]; NacCar | 20X; 5mM | -1.314 | Cilofexor | 2.000 | 0.195 |
| 21 | 1 | Lipid | LIRQS; NacCar | 20X; 5mM | -0.349 | Cilofexor | 0.031 | 0.019 |
| 22 | 1 | Lipid | LIRQS; NacCar | 20X; 5mM | -0.349 | Cilofexor | 0.125 | 0.072 |
| 23 | 1 | Lipid | LIRQS; NacCar | 20X; 5mM | -0.349 | Cilofexor | 0.500 | 0.027 |
| 24 | 1 | Lipid | LIRQS; NacCar | 20X; 5mM | -0.349 | Cilofexor | 2.000 | 0.195 |
| 25 | 1 | Lipid | LIRQ[2.5]S[10]; NacCar | 20X; 5mM | -1.314 | Tropifexor | 0.063 | 0.169 |
| 26 | 1 | Lipid | LIRQ[2.5]S[10]; NacCar | 20X; 5mM | -1.314 | Tropifexor | 0.250 | 0.143 |
| 27 | 1 | Lipid | LIRQ[2.5]S[10]; NacCar | 20X; 5mM | -1.314 | Tropifexor | 1.000 | 0.185 |
| 28 | 1 | Lipid | LIRQ[2.5]S[10]; NacCar | 20X; 5mM | -1.314 | Tropifexor | 4.000 | 0.214 |
| 29 | 1 | Lipid | LIRQS; NacCar | 20X; 5mM | -0.349 | Tropifexor | 0.063 | 0.169 |
| 30 | 1 | Lipid | LIRQS; NacCar | 20X; 5mM | -0.349 | Tropifexor | 0.250 | 0.143 |
| 31 | 1 | Lipid | LIRQS; NacCar | 20X; 5mM | -0.349 | Tropifexor | 1.000 | 0.185 |
| 32 | 1 | Lipid | LIRQS; NacCar | 20X; 5mM | -0.349 | Tropifexor | 4.000 | 0.214 |
| Row | Combination [AA+Supp.+Rx] | | | Combination effect | | Sum [AA+Supp.+Rx] effect | | Synergy Score (Combo. Effect — Sum [AA+supp.+Rx] effect) |
| 1 | Selonsertib+LIRQ[2.5]S[10]; NacCar | | | -1.569 | | -1.485 | | -0.084 |
| 2 | Selonsertib+LIRQ[2.5]S[10]; NacCar | | | -1.827 | | -1.470 | | -0.357 |
| 3 | Selonsertib+LIRQ[2.5]S[10]; NacCar | | | -1.917 | | -1.579 | | -0.338 |
| 4 | Selonsertib+LIRQ[2.5]S[10]; NacCar | | | -1.734 | | -1.577 | | -0.157 |
| 5 | Selonsertib+LIRQS; NacCar | | | -0.525 | | -0.472 | | -0.053 |
| 6 | Selonsertib+LIRQS; NacCar | | | -0.549 | | -0.457 | | -0.092 |
| 7 | Selonsertib+LIRQS; NacCar | | | -0.650 | | -0.565 | | -0.084 |
| 8 | Selonsertib+LIRQS; NacCar | | | -0.509 | | -0.564 | | 0.055 |
| 9 | Firsocostat+LIRQ[2.5]S[10]; NacCar | | | -1.983 | | -1.635 | | -0.348 |
| 10 | Firsocostat+LIRQ[2.5]S[10]; NacCar | | | -2.079 | | -1.709 | | -0.370 |
| 11 | Firsocostat+LIRQ[2.5]S[10]; NacCar | | | -2.312 | | -1.837 | | -0.474 |
| 12 | Firsocostat+LIRQ[2.5]S[10]; NacCar | | | -2.299 | | -1.742 | | -0.557 |
| 13 | Firsocostat+LIRQS; NacCar | | | -1.010 | | -0.621 | | -0.389 |
| 14 | Firsocostat+LIRQS; NacCar | | | -1.397 | | -0.696 | | -0.702 |
| 15 | Firsocostat+LIRQS; NacCar | | | -1.716 | | -0.824 | | -0.893 |
| 16 | Firsocostat+LIRQS; NacCar | | | -1.808 | | -0.729 | | -1.079 |
| 17 | Cilofexor+LIRQ[2.5]S[10]; NacCar | | | -1.648 | | -1.295 | | -0.353 |
| 18 | Cilofexor+LIRQ[2.5]S[10]; NacCar | | | -1.489 | | -1.242 | | -0.247 |
| 19 | Cilofexor+LIRQ[2.5]S[10]; NacCar | | | -1.518 | | -1.288 | | -0.230 |
| 20 | Cilofexor+LIRQ[2.5]S[10]; NacCar | | | -1.200 | | -1.120 | | -0.080 |
| 21 | Cilofexor+LIRQS; NacCar | | | -0.459 | | -0.329 | | -0.130 |
| 22 | Cilofexor+LIRQS; NacCar | | | -0.476 | | -0.277 | | -0.199 |
| 23 | Cilofexor+LIRQS; NacCar | | | -0.276 | | -0.322 | | 0.046 |
| 24 | Cilofexor+LIRQS; NacCar | | | -0.064 | | -0.154 | | 0.090 |
| 25 | Tropifexor+LIRQ[2.5]S[10]; NacCar | | | -0.990 | | -1.145 | | 0.156 |
| 26 | Tropifexor+LIRQ[2.5]S[10]; NacCar | | | -0.975 | | -1.171 | | 0.197 |
| 27 | Tropifexor+LIRQ[2.5]S[10]; NacCar | | | -1.093 | | -1.129 | | 0.037 |
| 28 | Tropifexor+LIRQ[2.5]S[10]; NacCar | | | -0.953 | | -1.101 | | 0.147 |
| 29 | Tropifexor+LIRQS; NacCar | | | 0.061 | | -0.180 | | 0.241 |
| 30 | Tropifexor+LIRQS; NacCar | | | 0.069 | | -0.205 | | 0.275 |
| 31 | Tropifexor+LIRQS; NacCar | | | 0.041 | | -0.164 | | 0.204 |
| 32 | Tropifexor+LIRQS; NacCar | | | 0.091 | | -0.135 | | 0.226 |

TABLE E2

| Row | Donor | phenotype | Amino Acid | AA Conc. (X)* | AA+Supp. effect | Rx | Rx Conc. (µM) | Rx effect |
|---|---|---|---|---|---|---|---|---|
| | | | Supplement | Nac/Car (mM) | | | | |
| 1 | 2 | Lipid | LIRQ[2.5]S[10]; NacCar | 20X; 5mM | -1.010 | Selonsertib | 0.063 | -0.096 |
| 2 | 2 | Lipid | LIRQ[2.5]S[10]; NacCar | 20X; 5mM | -1.010 | Selonsertib | 0.250 | -0.059 |
| 3 | 2 | Lipid | LIRQ[2.5]S[10]; NacCar | 20X; 5mM | -1.010 | Selonsertib | 1.000 | -0.164 |
| 4 | 2 | Lipid | LIRQ[2.5]S[10]; NacCar | 20X; 5mM | -1.010 | Selonsertib | 4.000 | -0.307 |
| 5 | 2 | Lipid | LIRQS; NacCar | 20X; 5mM | -1.011 | Selonsertib | 0.063 | -0.096 |
| 6 | 2 | Lipid | LIRQS; NacCar | 20X; 5mM | -1.011 | Selonsertib | 0.250 | -0.059 |
| 7 | 2 | Lipid | LIRQS; NacCar | 20X; 5mM | -1.011 | Selonsertib | 1.000 | -0.164 |
| 8 | 2 | Lipid | LIRQS; NacCar | 20X; 5mM | -1.011 | Selonsertib | 4.000 | -0.307 |
| 9 | 2 | Lipid | LIRQ[2.5]S[10]; NacCar | 20X; 5mM | -1.010 | Firsocostat | 0.063 | -0.246 |
| 10 | 2 | Lipid | LIRQ[2.5]S[10]; NacCar | 20X; 5mM | -1.010 | Firsocostat | 0.250 | -0.290 |
| 11 | 2 | Lipid | LIRQ[2.5]S[10]; NacCar | 20X; 5mM | -1.010 | Firsocostat | 1.000 | -0.449 |
| 12 | 2 | Lipid | LIRQ[2.5]S[10]; NacCar | 20X; 5mM | -1.010 | Firsocostat | 4.000 | -0.485 |
| 13 | 2 | Lipid | LIRQS; NacCar | 20X; 5mM | -1.011 | Firsocostat | 0.063 | -0.246 |
| 14 | 2 | Lipid | LIRQS; NacCar | 20X; 5mM | -1.011 | Firsocostat | 0.250 | -0.290 |
| 15 | 2 | Lipid | LIRQS; NacCar | 20X; 5mM | -1.011 | Firsocostat | 1.000 | -0.449 |
| 16 | 2 | Lipid | LIRQS; NacCar | 20X; 5mM | -1.011 | Firsocostat | 4.000 | -0.485 |
| 17 | 2 | Lipid | LIRQ[2.5]S[10]; NacCar | 20X; 5mM | -0.988 | Cilofexor | 0.031 | -0.049 |
| 18 | 2 | Lipid | LIRQ[2.5]S[10]; NacCar | 20X; 5mM | -0.988 | Cilofexor | 0.125 | 0.031 |
| 19 | 2 | Lipid | LIRQ[2.5]S[10]; NacCar | 20X; 5mM | -0.988 | Cilofexor | 0.500 | 0.104 |
| 20 | 2 | Lipid | LIRQ[2.5]S[10]; NacCar | 20X; 5mM | -0.988 | Cilofexor | 2.000 | 0.050 |
| 21 | 2 | Lipid | LIRQS; NacCar | 20X; 5mM | -0.831 | Cilofexor | 0.031 | -0.049 |
| 22 | 2 | Lipid | LIRQS; NacCar | 20X; 5mM | -0.831 | Cilofexor | 0.125 | 0.031 |
| 23 | 2 | Lipid | LIRQS; NacCar | 20X; 5mM | -0.831 | Cilofexor | 0.500 | 0.104 |
| 24 | 2 | Lipid | LIRQS; NacCar | 20X; 5mM | -0.831 | Cilofexor | 2.000 | 0.050 |
| 25 | 2 | Lipid | LIRQ[2.5]S[10]; NacCar | 20X; 5mM | -0.988 | Tropifexor | 0.063 | 0.132 |
| 26 | 2 | Lipid | LIRQ[2.5]S[10]; NacCar | 20X; 5mM | -0.988 | Tropifexor | 0.250 | 0.161 |
| 27 | 2 | Lipid | LIRQ[2.5]S[10]; NacCar | 20X; 5mM | -0.988 | Tropifexor | 1.000 | 0.083 |
| 28 | 2 | Lipid | LIRQ[2.5]S[10]; NacCar | 20X; 5mM | -0.988 | Tropifexor | 4.000 | 0.049 |
| 29 | 2 | Lipid | LIRQS; NacCar | 20X; 5mM | -0.831 | Tropifexor | 0.063 | 0.132 |
| 30 | 2 | Lipid | LIRQS; NacCar | 20X; 5mM | -0.831 | Tropifexor | 0.250 | 0.161 |
| 31 | 2 | Lipid | LIRQS; NacCar | 20X; 5mM | -0.831 | Tropifexor | 1.000 | 0.083 |
| 32 | 2 | Lipid | LIRQS; NacCar | 20X; 5mM | -0.831 | Tropifexor | 4.000 | 0.049 |

| | | | | |
|---|---|---|---|---|
| Row | Combination [AA+Supp.+Rx] | Combination effect | Sum [AA+Supp.+Rx] effect | Synergy Score (Combo. Effect — Sum [AA+Supp.+Rx] effect) |
| 1 | Selonsertib+LIRQ[2.5]S[10]; NacCar | -1.170 | -1.105 | -0.065 |
| 2 | Selonsertib+LIRQ[2.5]S[10]; NacCar | -1.311 | -1.069 | -0.242 |
| 3 | Selonsertib+LIRQ[2.5]S[10]; NacCar | -1.646 | -1.173 | -0.473 |
| 4 | Selonsertib+LIRQ[2.5]S[10]; NacCar | -2.013 | -1.316 | -0.696 |
| 5 | Selonsertib+LIRQS; NacCar | -1.085 | -1.106 | 0.021 |
| 6 | Selonsertib+LIRQS; NacCar | -1.245 | -1.069 | -0.176 |
| 7 | Selonsertib+LIRQS; NacCar | -1.465 | -1.174 | -0.291 |
| 8 | Selonsertib+LIRQS; NacCar | -1.555 | -1.317 | -0.238 |
| 9 | Firsocostat+LIRQ[2.5]S[10]; NacCar | -1.956 | -1.255 | -0.700 |
| 10 | Firsocostat+LIRQ[2.5]S[10]; NacCar | -2.365 | -1.300 | -1.065 |
| 11 | Firsocostat+LIRQ[2.5]S[10]; NacCar | -2.944 | -1.459 | -1.485 |
| 12 | Firsocostat+LIRQ[2.5]S[10]; NacCar | -3.046 | -1.495 | -1.551 |
| 13 | Firsocostat+LIRQS; NacCar | -1.765 | -1.256 | -0.509 |
| 14 | Firsocostat+LIRQS; NacCar | -2.312 | -1.301 | -1.012 |
| 15 | Firsocostat+LIRQS; NacCar | -2.769 | -1.460 | -1.309 |
| 16 | Firsocostat+LIRQS; NacCar | -2.840 | -1.496 | -1.344 |
| 17 | Cilofexor+LIRQ[2.5]S[10]; NacCar | -0.890 | -1.037 | 0.148 |
| 18 | Cilofexor+LIRQ[2.5]S[10]; NacCar | -0.976 | -0.957 | -0.019 |
| 19 | Cilofexor+LIRQ[2.5]S[10]; NacCar | -1.021 | -0.884 | -0.136 |
| 20 | Cilofexor+LIRQ[2.5]S[10]; NacCar | -0.901 | -0.938 | 0.037 |
| 21 | Cilofexor+LIRQS; NacCar | -0.856 | -0.879 | 0.024 |
| 22 | Cilofexor+LIRQS; NacCar | -0.762 | -0.799 | 0.037 |
| 23 | Cilofexor+LIRQS; NacCar | -0.883 | -0.726 | -0.157 |
| 24 | Cilofexor+LIRQS; NacCar | -0.855 | -0.780 | -0.075 |
| 25 | Tropifexor+LIRQ[2.5]S[10]; NacCar | -0.768 | -0.857 | 0.088 |
| 26 | Tropifexor+LIRQ[2.5]S[10]; NacCar | -0.987 | -0.828 | -0.159 |
| 27 | Tropifexor+LIRQ[2.5]S[10]; NacCar | -1.006 | -0.906 | -0.100 |
| 28 | Tropifexor+LIRQ[2.5]S[10]; NacCar | -1.051 | -0.939 | -0.112 |
| 29 | Tropifexor+LIRQS; NacCar | -0.604 | -0.699 | 0.095 |
| 30 | Tropifexor+LIRQS; NacCar | -0.686 | -0.670 | -0.016 |
| 31 | Tropifexor+LIRQS; NacCar | -0.661 | -0.748 | 0.087 |
| 32 | Tropifexor+LIRQS; NacCar | -0.583 | -0.781 | 0.198 |

*X corresponds to the amino acid concentration values relative to the mean physiological concentrations in blood HMDB. The values are published in the Human Metabolome Database (HMDB). Q[2.5] and S[10] in the composition LIR[Q2.5]S[10]; NacCar were used respectively at 2.5X and 10X relative to 1X HMDB.

### MCP1/CCL2 Secretion

Tables E3 and E4 show the level of MCP1/CCL2 secretion subtracted from the baseline (sFA+TNF-α) and normalized to nuclei in primary human hepatocytes cells from two healthy donors 1 and 2 respectively. Treatment with LIRQSNacCar and LIRQ[2.5]S[10]NacCar at 20X reduce MCP1 level compared to baseline (Table E3 & E4, column 5). Treatment with Selonsertib and Firsocostat shows a slight reduction in MCP1 secretion or a slight increase when treated with Cilofexor and Tropifexor compared to the baseline (Table E3 & E4, column 8).

Similar to lipid, reducing MCP1 secretion is the desired phenotype, a negative synergy score corresponds to a stronger reduction of MCP1 in the Rx-AA composition combination compared to the individual treatments. Treatment with LIRQSNacCar or with LIRQ[2.5]S[10]NacCar in combination with either Firsocostat, Selonsertib or Cilofexor show a synergy score that is lower than 0 at different doses of the drug suggesting that the combination Rx-AA composition reduces MCP1 secretion at a higher level than the Rx or the AA composition alone. Treatment with Tropifexor in combination with LIRQSNacCar or with LIRQ[2.5]S[10]NacCar show a synergy score higher than 0 at all drug doses in donor 1 and almost all the drug doses in donor 2 suggesting that the combination Tropifexor-AA composition induces MCP1 secretion at a higher level than Tropifexor or LIRQSNacCar or LIRQ[2.5]S[10]NacCar alone.

TABLE E3

| Row | Donor | phenotype | Amino Acid | AA Conc. (X)* | AA+Supp. effect | Rx | Rx Conc. (µM) | Rx effect |
|---|---|---|---|---|---|---|---|---|
| | | | Supplement | Nac/Car (mM) | | | | |
| 1 | 1 | MCP1 | LIRQ[2.5]S[10]; NacCar | 20X; 5mM | -2.427 | Selonsertib | 0.063 | -0.167 |
| 2 | 1 | MCP1 | LIRQ[2.5]S[10]; NacCar | 20X; 5mM | -2.427 | Selonsertib | 0.250 | -0.126 |
| 3 | 1 | MCP1 | LIRQ[2.5]S[10]; NacCar | 20X; 5mM | -2.427 | Selonsertib | 1.000 | -0.503 |
| 4 | 1 | MCP1 | LIRQ[2.5]S[10]; NacCar | 20X; 5mM | -2.427 | Selonsertib | 4.000 | -0.552 |
| 5 | 1 | MCP1 | LIRQS; NacCar | 20X; 5mM | -3.111 | Selonsertib | 0.063 | -0.167 |
| 6 | 1 | MCP1 | LIRQS; NacCar | 20X; 5mM | -3.111 | Selonsertib | 0.250 | -0.126 |
| 7 | 1 | MCP1 | LIRQS; NacCar | 20X; 5mM | -3.111 | Selonsertib | 1.000 | -0.503 |
| 8 | 1 | MCP1 | LIRQS; NacCar | 20X; 5mM | -3.111 | Selonsertib | 4.000 | -0.552 |
| 9 | 1 | MCP1 | LIRQ[2.5]S[10]; NacCar | 20X; 5mM | -2.427 | Firsocostat | 0.063 | -0.233 |
| 10 | 1 | MCP1 | LIRQ[2.5]S[10]; NacCar | 20X; 5mM | -2.427 | Firsocostat | 0.250 | -0.460 |
| 11 | 1 | MCP1 | LIRQ[2.5]S[10]; NacCar | 20X; 5mM | -2.427 | Firsocostat | 1.000 | -0.827 |
| 12 | 1 | MCP1 | LIRQ[2.5]S[10]; NacCar | 20X; 5mM | -2.427 | Firsocostat | 4.000 | -0.479 |
| 13 | 1 | MCP1 | LIRQS; NacCar | 20X; 5mM | -3.111 | Firsocostat | 0.063 | -0.233 |
| 14 | 1 | MCP1 | LIRQS; NacCar | 20X; 5mM | -3.111 | Firsocostat | 0.250 | -0.460 |
| 15 | 1 | MCP1 | LIRQS; NacCar | 20X; 5mM | -3.111 | Firsocostat | 1.000 | -0.827 |
| 16 | 1 | MCP1 | LIRQS; NacCar | 20X; 5mM | -3.111 | Firsocostat | 4.000 | -0.479 |
| 17 | 1 | MCP1 | LIRQ[2.5]S[10]; NacCar | 20X; 5mM | -2.628 | Cilofexor | 0.031 | -0.009 |
| 18 | 1 | MCP1 | LIRQ[2.5]S[10]; NacCar | 20X; 5mM | -2.628 | Cilofexor | 0.125 | 0.582 |
| 19 | 1 | MCP1 | LIRQ[2.5]S[10]; NacCar | 20X; 5mM | -2.628 | Cilofexor | 0.500 | 1.210 |
| 20 | 1 | MCP1 | LIRQ[2.5]S[10]; NacCar | 20X; 5mM | -2.628 | Cilofexor | 2.000 | 1.898 |
| 21 | 1 | MCP1 | LIRQS; NacCar | 20X; 5mM | -2.630 | Cilofexor | 0.031 | -0.009 |
| 22 | 1 | MCP1 | LIRQS; NacCar | 20X; 5mM | -2.630 | Cilofexor | 0.125 | 0.582 |
| 23 | 1 | MCP1 | LIRQS; NacCar | 20X; 5mM | -2.630 | Cilofexor | 0.500 | 1.210 |
| 24 | 1 | MCP1 | LIRQS; NacCar | 20X; 5mM | -2.630 | Cilofexor | 2.000 | 1.898 |
| 25 | 1 | MCP1 | LIRQ[2.5]S[10]; NacCar | 20X; 5mM | -2.628 | Tropifexor | 0.063 | 0.956 |
| 26 | 1 | MCP1 | LIRQ[2.5]S[10]; NacCar | 20X; 5mM | -2.628 | Tropifexor | 0.250 | 0.762 |
| 27 | 1 | MCP1 | LIRQ[2.5]S[10]; NacCar | 20X; 5mM | -2.628 | Tropifexor | 1.000 | 0.877 |
| 28 | 1 | MCP1 | LIRQ[2.5]S[10]; NacCar | 20X; 5mM | -2.628 | Tropifexor | 4.000 | 0.658 |
| 29 | 1 | MCP1 | LIRQS; NacCar | 20X; 5mM | -2.630 | Tropifexor | 0.063 | 0.956 |
| 30 | 1 | MCP1 | LIRQS; NacCar | 20X; 5mM | -2.630 | Tropifexor | 0.250 | 0.762 |
| 31 | 1 | MCP1 | LIRQS; NacCar | 20X; 5mM | -2.630 | Tropifexor | 1.000 | 0.877 |
| 32 | 1 | MCP1 | LIRQS; NacCar | 20X; 5mM | -2.630 | Tropifexor | 4.000 | 0.658 |

| | | | | |
|---|---|---|---|---|
| Row | Combination [AA+Supp.+Rx] | Combination effect | Sum [AA+Supp.+Rx] effect | Synergy Score (Combo. Effect — Sum [AA+Supp.+Rx] effect) |
| 1 | Selonsertib+LIRQ[2.5]S[10]; NacCar | -2.642 | -2.594 | -0.048 |
| 2 | Selonsertib+LIRQ[2.5]S[10]; NacCar | -3.092 | -2.553 | -0.538 |
| 3 | Selonsertib+LIRQ[2.5]S[10]; NacCar | -3.237 | -2.930 | -0.307 |
| 4 | Selonsertib+LIRQ[2.5]S[10]; NacCar | -3.215 | -2.979 | -0.236 |
| 5 | Selonsertib+LIRQS; NacCar | -3.287 | -3.278 | -0.009 |
| 6 | Selonsertib+LIRQS; NacCar | -3.554 | -3.238 | -0.317 |
| 7 | Selonsertib+LIRQS; NacCar | -3.517 | -3.614 | 0.097 |
| 8 | Selonsertib+LIRQS; NacCar | -3.494 | -3.663 | 0.169 |
| 9 | Firsocostat+LIRQ[2.5]S[10]; NacCar | -3.003 | -2.660 | -0.342 |
| 10 | Firsocostat+LIRQ[2.5]S[10]; NacCar | -3.431 | -2.887 | -0.544 |
| 11 | Firsocostat+LIRQ[2.5]S[10]; NacCar | -3.212 | -3.254 | 0.042 |
| 12 | Firsocostat+LIRQ[2.5]S[10]; NacCar | -3.097 | -2.906 | -0.191 |
| 13 | Firsocostat+LIRQS; NacCar | -3.561 | -3.344 | -0.217 |
| 14 | Firsocostat+LIRQS; NacCar | -3.815 | -3.571 | -0.244 |
| 15 | Firsocostat+LIRQS; NacCar | -3.827 | -3.938 | 0.111 |
| 16 | Firsocostat+LIRQS; NacCar | -3.697 | -3.590 | -0.107 |
| 17 | Cilofexor+LIRQ[2.5]S[10]; NacCar | -2.399 | -2.637 | 0.238 |
| 18 | Cilofexor+LIRQ[2.5]S[10]; NacCar | -2.478 | -2.046 | -0.433 |
| 19 | Cilofexor+LIRQ[2.5]S[10]; NacCar | -1.744 | -1.418 | -0.326 |
| 20 | Cilofexor+LIRQ[2.5]S[10]; NacCar | -0.929 | -0.730 | -0.199 |
| 21 | Cilofexor+LIRQS; NacCar | -3.213 | -2.639 | -0.574 |
| 22 | Cilofexor+LIRQS; NacCar | -3.760 | -2.047 | -1.712 |
| 23 | Cilofexor+LIRQS; NacCar | -2.459 | -1.419 | -1.040 |
| 24 | Cilofexor+LIRQS; NacCar | -1.363 | -0.732 | -0.631 |
| 25 | Tropifexor+LIRQ[2.5]S[10]; NacCar | -0.886 | -1.672 | 0.785 |
| 26 | Tropifexor+LIRQ[2.5]S[10]; NacCar | -1.207 | -1.866 | 0.659 |
| 27 | Tropifexor+LIRQ[2.5]S[10]; NacCar | -0.982 | -1.750 | 0.768 |
| 28 | Tropifexor+LIRQ[2.5]S[10]; NacCar | -0.873 | -1.970 | 1.097 |
| 29 | Tropifexor+LI RQS; NacCar | -1.594 | -1.673 | 0.080 |
| 30 | Tropifexor+LI RQS; NacCar | -1.601 | -1.867 | 0.267 |
| 31 | Tropifexor+LI RQS; NacCar | -1.443 | -1.752 | 0.309 |
| 32 | Tropifexor+LI RQS; NacCar | -1.617 | -1.971 | 0.355 |

TABLE E4

| Row | Donor | phenotype | Amino Acid | AA Conc. (X)* | AA+Supp. effect | Rx | Rx Conc. (µM) | Rx effect |
|---|---|---|---|---|---|---|---|---|
| | | | Supplement | Nac/Car (mM) | | | | |
| 1 | 2 | MCP1 | LIRQ[2.5]S[10]; NacCar | 20X; 5mM | -0.533 | Selonsertib | 0.063 | -0.306 |
| 2 | 2 | MCP1 | LIRQ[2.5]S[10]; NacCar | 20X; 5mM | -0.533 | Selonsertib | 0.250 | -0.083 |
| 3 | 2 | MCP1 | LIRQ[2.5]S[10]; NacCar | 20X; 5mM | -0.533 | Selonsertib | 1.000 | -0.152 |
| 4 | 2 | MCP1 | LIRQ[2.5]S[10]; NacCar | 20X; 5mM | -0.533 | Selonsertib | 4.000 | -0.462 |
| 5 | 2 | MCP1 | LIRQS; NacCar | 20X; 5mM | -1.838 | Selonsertib | 0.063 | -0.306 |
| 6 | 2 | MCP1 | LIRQS; NacCar | 20X; 5mM | -1.838 | Selonsertib | 0.250 | -0.083 |
| 7 | 2 | MCP1 | LIRQS; NacCar | 20X; 5mM | -1.838 | Selonsertib | 1.000 | -0.152 |
| 8 | 2 | MCP1 | LIRQS; NacCar | 20X; 5mM | -1.838 | Selonsertib | 4.000 | -0.462 |
| 9 | 2 | MCP1 | LIRQ[2.5]S[10]; NacCar | 20X; 5mM | -0.533 | Firsocostat | 0.063 | -0.319 |
| 10 | 2 | MCP1 | LIRQ[2.5]S[10]; NacCar | 20X; 5mM | -0.533 | Firsocostat | 0.250 | -0.360 |
| 11 | 2 | MCP1 | LIRQ[2.5]S[10]; NacCar | 20X; 5mM | -0.533 | Firsocostat | 1.000 | -0.614 |
| 12 | 2 | MCP1 | LIRQ[2.5]S[10]; NacCar | 20X; 5mM | -0.533 | Firsocostat | 4.000 | -0.775 |
| 13 | 2 | MCP1 | LIRQS; NacCar | 20X; 5mM | -1.838 | Firsocostat | 0.063 | -0.319 |
| 14 | 2 | MCP1 | LIRQS; NacCar | 20X; 5mM | -1.838 | Firsocostat | 0.250 | -0.360 |
| 15 | 2 | MCP1 | LIRQS; NacCar | 20X; 5mM | -1.838 | Firsocostat | 1.000 | -0.614 |
| 16 | 2 | MCP1 | LIRQS; NacCar | 20X; 5mM | -1.838 | Firsocostat | 4.000 | -0.775 |
| 17 | 2 | MCP1 | LIRQ[2.5]S[10]; NacCar | 20X; 5mM | 0.054 | Cilofexor | 0.031 | -0.028 |
| 18 | 2 | MCP1 | LIRQ[2.5]S[10]; NacCar | 20X; 5mM | 0.054 | Cilofexor | 0.125 | 0.28 |
| 19 | 2 | MCP1 | LIRQ[2.5]S[10]; NacCar | 20X; 5mM | 0.054 | Cilofexor | 0.500 | 0.47 |
| 20 | 2 | MCP1 | LIRQ[2.5]S[10]; NacCar | 20X; 5mM | 0.054 | Cilofexor | 2.000 | 0.69 |
| 21 | 2 | MCP1 | LIRS; NacCar | 20X 5mM | -1.165 | Cilofexor | 0.031 | -0.02 |
| 22 | 2 | MCP1 | LIRQS; NacCar | 20X; 5mM | -1.165 | Cilofexor | 0.125 | 0.28 |
| 23 | 2 | MCP1 | LIRQS; NacCar | 20X; 5mM | -1.165 | Cilofexor | 0.500 | 0.47 |
| 24 | 2 | MCP1 | LIRQS; NacCar | 20X; 5mM | -1.165 | Cilofexor | 2.000 | 0.695 |
| 25 | 2 | MCP1 | LIRQ[2.5]S[10]; NacCar | 20X; 5mM | 0.054 | Tropifexor | 0.063 | -0.160 |
| 26 | 2 | MCP1 | LIRQ[2.5]S[10]; NacCar | 20X; 5mM | 0.054 | Tropifexor | 0.250 | -0.250 |
| 27 | 2 | MCP1 | LIRQ[2.5]S[10]; NacCar | 20X; 5mM | 0.054 | Tropifexor | 1.000 | -0.43 |
| 28 | 2 | MCP1 | LIRQ[2.5]S[10]; NacCar | 20X; 5mM | 0.054 | Tropifexor | 4.000 | -0.65 |
| 29 | 2 | MCP1 | LIRQS; NacCar | 20X; 5mM | -1.165 | Tropifexor | 0.063 | -0.16 |
| 30 | 2 | MCP1 | LIRQS; NacCar | 20X; 5mM | -1.165 | Tropifexor | 0.250 | -0.256 |
| 31 | 2 | MCP1 | LIRQS; NacCar | 20X; 5mM | -1.165 | Tropifexor | 1.000 | -0.433 |
| 32 | 2 | MCP1 | LIRQS; NacCar | 20X; 5mM | -1.165 | Tropifexor | 4.000 | -0.659 |

| | | | | |
|---|---|---|---|---|
| Row | Combination [AA+Supp.+Rx] | Combination effect | Sum [AA+Supp.+Rx] effect | Synery Score (Combo. Effect — Sum [AA+Supp.+Rx] effect) |
| 1 | Selonsertib+LIRQ[2.5]S[10]; NacCar | -0.806 | -0.839 | 0.033 |
| 2 | Selonsertib+LIRQ[2.5]S[10]; NacCar | -0.850 | -0.616 | -0.234 |
| 3 | Selonsertib+LIRQ[2.5]S[10]; NacCar | -0.665 | -0.685 | 0.020 |
| 4 | Selonsertib+LIRQ[2.5]S[10]; NacCar | -1.127 | -0.995 | -0.132 |
| 5 | Selonsertib+LIRQS; NacCar | -1.911 | -2.144 | 0.233 |
| 6 | Selonsertib+LIRQS; NacCar | -2.023 | -1.922 | -0.101 |
| 7 | Selonsertib+LIRQS; NacCar | -2.001 | -1.990 | -0.010 |
| 8 | Selonsertib+LIRQS; NacCar | -2.227 | -2.300 | 0.073 |
| 9 | Firsocostat+LIRQ[2.5]S[10]; NacCar | -0.833 | -0.853 | 0.019 |
| 10 | Firsocostat+LIRQ[2.5]S[10]; NacCar | -0.871 | -0.893 | 0.022 |
| 11 | Firsocostat+LIRQ[2.5]S[10]; NacCar | -1.482 | -1.147 | -0.335 |
| 12 | Firsocostat+LIRQ[2.5]S[10]; NacCar | -1.654 | -1.308 | -0.34 |
| 13 | Firsocostat+LIRQS; NacCar | -2.113 | -2.158 | 0.04 |
| 14 | Firsocostat+LIRQS; NacCar | -2.169 | -2.198 | 0.020 |
| 15 | Firsocostat+LIRQS; NacCar | -2.527 | -2.452 | -0.07 |
| 16 | Firsocostat+LIRQS; NacCar | -2.858 | -2.613 | -0.24 |
| 17 | Cilofexor+LIRQ[2.5]S[10]; NacCar | -0.609 | 0.026 | -0.63 |
| 18 | Cilofexor+LIRQ[2.5]S[10]; NacCar | -0.532 | 0.343 | -0.875 |
| 19 | Cilofexor+LIRQ[2.5]S[10]; NacCar | -0.408 | 0.532 | -0.94 |
| 20 | Cilofexor+LIRQ[2.5]S[10]; NacCar | 0.055 | 0.750 | -0.69 |
| 21 | Cilofexor+LIRQS; NacCar | -1.969 | -1.193 | -0.77 |
| 22 | Cilofexor+LIRQS; NacCar | -2.070 | -0.876 | -1.19 |
| 23 | Cilofexor+LIRQS; NacCar | -1.539 | -0.687 | -0.85 |
| 24 | Cilofexor+LIRQS; NacCar | -0.550 | -0.470 | -0.081 |
| 25 | Tropifexor+LIRQ[2.5]S[10]; NacCar | -0.052 | -0.110 | 0.057 |
| 26 | Tropifexor+LIRQ[2.5]S[10]; NacCar | -0.853 | -0.202 | -0.651 |
| 27 | Tropifexor+LIRQ[2.5]S[10]; NacCar | -0.847 | -0.378 | -0.469 |
| 28 | Tropifexor+LIRQ[2.5]S[10]; NacCar | -1.062 | -0.605 | -0.458 |
| 29 | Tropifexor+LIRQS; NacCar | -1.038 | -1.329 | 0.291 |
| 30 | Tropifexor+LIRQS; NacCar | -1.346 | -1.421 | 0.074 |
| 31 | Tropifexor+LIRQS; NacCar | -1.107 | -1.597 | 0.490 |
| 32 | Tropifexor+LIRQS; NacCar | -1.463 | -1.824 | 0.361 |

### Results 2 (Donor 2)

### Lipid Accumulation and Steatosis Phenotypes

Table E5 shows the level of total lipid area subtracted from the baseline (sFA+TNFa) and normalized to nuclei in primary human hepatocytes cells from one healthy donor (donor 2). Treatment with LIRQ[2.5]S[10]NacCar or NacCar reduced lipid level compared to baseline (Table E5, column 5). Treatment with the therapeutic agents (Rx) shows a slight reduction in lipid level when treated with Firsocostat and Selonsertib or no changes in lipid level when treated with Cilofexor (Table E1 & E2, column 8). In addition, the impact on lipid of the combination of each of the compositions LIRQ[2.5]S[10]NacCar or NacCar with 4 different doses of the Rx (Selonsertib, Firsocostat and Cilofexor) was measured. The effect of the combination Rx- LIRQ[2.5]S[10]NacCar or Rx-NacCar on lipid was evaluated by calculating a synergy score. As previously described in Result 1, a negative synergy score corresponds to a stronger reduction of lipid level in the combination compared to the singles. Treatment with either Firsocostat, Selonsertib or Cilofexor in combination with either LIRQ[2.5]S[10]NacCar or NacCar show a negative synergy score suggesting that the combination Rx-LIRQ[2.5]S[10]NacCar, or Rx-NacCar reduces lipid at a higher level than the Rx (Firsocostat, Selonsertib, Cilofexor), LIRQ[2.5]S[10]NacCar or NacCar alone.

TABLE E5

| Row | Donor | phenotype | Amino Acid | AA Conc. (X)* | AA+Supp. effect | Rx | Rx Conc. (µM) | Rx effect |
|---|---|---|---|---|---|---|---|---|
| | | | Supplement (Supp.) | Nac/Car (mM) | | | | |
| 1 | 2 | Lipid | LIRQ[2.5]S[10]; NacCar | 20X; 5 | -0.864 | Selonsertib | 0.0625 | -0.024 |
| 2 | 2 | Lipid | LIRQ[2.5]S[10]; NacCar | 20X; 5 | -0.864 | Selonsertib | 0.25 | -0.067 |
| 3 | 2 | Lipid | LIRQ[2.5]S[10]; NacCar | 20X; 5 | -0.864 | Selonsertib | 1 | -0.156 |
| 4 | 2 | Lipid | LIRQ[2.5]S[10]; NacCar | 20X; 5 | -0.864 | Selonsertib | 4 | -0.178 |
| 5 | 2 | Lipid | NacCar | 5 | -1.600 | Selonsertib | 0.0625 | -0.024 |
| 6 | 2 | Lipid | NacCar | 5 | -1.600 | Selonsertib | 0.25 | -0.067 |
| 7 | 2 | Lipid | NacCar | 5 | -1.600 | Selonsertib | 1 | -0.156 |
| 8 | 2 | Lipid | NacCar | 5 | -1.600 | Selonsertib | 4 | -0.178 |
| 9 | 2 | Lipid | LIRQ[2.5]S[10]; NacCar | 20X; 5 | -0.864 | Firsocostat | 0.0625 | -0.147 |
| 10 | 2 | Lipid | LIRQ[2.5]S[10]; NacCar | 20X; 5 | -0.864 | Firsocostat | 0.25 | -0.293 |
| 11 | 2 | Lipid | LIRQ[2.5]S[10]; NacCar | 20X; 5 | -0.864 | Firsocostat | 1 | -0.404 |
| 12 | 2 | Lipid | LIRQ[2.5]S[10]; NacCar | 20X; 5 | -0.864 | Firsocostat | 4 | -0.458 |
| 13 | 2 | Lipid | NacCar | 5 | -1.600 | Firsocostat | 0.0625 | -0.147 |
| 14 | 2 | Lipid | NacCar | 5 | -1.600 | Firsocostat | 0.25 | -0.293 |
| 15 | 2 | Lipid | NacCar | 5 | -1.600 | Firsocostat | 1 | -0.404 |
| 16 | 2 | Lipid | NacCar | 5 | -1.600 | Firsocostat | 4 | -0.458 |
| 17 | 2 | Lipid | LIRQ[2.5]S[10]; NacCar | 20X; 5 | -0.864 | Cilofexor | 0.0313 | -0.110 |
| 18 | 2 | Lipid | LIRQ[2.5]S[10]; NacCar | 20X; 5 | -0.864 | Cilofexor | 0.125 | 0.001 |
| 19 | 2 | Lipid | LIRQ[2.5]S[10]; NacCar | 20X; 5 | -0.864 | Cilofexor | 0.5 | 0.088 |
| 20 | 2 | Lipid | LIRQ[2.5]S[10]; NacCar | 20X; 5 | -0.864 | Cilofexor | 2 | 0.080 |
| 21 | 2 | Lipid | NacCar | 5 | -1.600 | Cilofexor | 0.0313 | -0.110 |
| 22 | 2 | Lipid | NacCar | 5 | -1.600 | Cilofexor | 0.125 | 0.001 |
| 23 | 2 | Lipid | NacCar | 5 | -1.600 | Cilofexor | 0.5 | 0.088 |
| 24 | 2 | Lipid | NacCar | 5 | -1.600 | Cilofexor | 2 | 0.080 |
| Row | Combination [AA+Supp.+Rx] | | | Combination effect | | Sum [AA+Supp.+Rx] effect | | Synergy Score (Combo. Effect — Sum [AA+Supp.+Rx] effect) |
| 1 | Selonsertib+LIRQ[2.5]S[10]; NacCar | | | -0.925 | | -0.888 | | -0.037 |
| 2 | Selonsertib+LIRQ[2.5]S[10]; NacCar | | | -1.146 | | -0.931 | | -0.215 |
| 3 | Selonsertib+LIRQ[2.5]S[10]; NacCar | | | -1.519 | | -1.020 | | -0.499 |
| 4 | Selonsertib+LIRQ[2.5]S[10]; NacCar | | | -1.732 | | -1.042 | | -0.690 |
| 5 | Selonsertib+NacCar | | | -1.801 | | -1.624 | | -0.177 |
| 6 | Selonsertib+NacCar | | | -1.918 | | -1.667 | | -0.251 |
| 7 | Selonsertib+NacCar | | | -1.961 | | -1.756 | | -0.205 |
| 8 | Selonsertib+NacCar | | | -2.275 | | -1.778 | | -0.497 |
| 9 | Firsocostat+LIRQ[2.5]S[10]; NacCar | | | -2.090 | | -1.011 | | -1.079 |
| 10 | Firsocostat+LIRQ[2.5]S[10]; NacCar | | | -2.387 | | -1.157 | | -1.230 |
| 11 | Firsocostat+LIRQ[2.5]S[10]; NacCar | | | -2.923 | | -1.268 | | -1.655 |
| 12 | Firsocostat+LIRQ[2.5]S[10]; NacCar | | | -2.650 | | -1.322 | | -1.327 |
| 13 | Firsocostat+NacCar | | | -2.351 | | -1.747 | | -0.604 |
| 14 | Firsocostat+NacCar | | | -2.778 | | -1.893 | | -0.885 |
| 15 | Firsocostat+NacCar | | | -3.041 | | -2.004 | | -1.038 |
| 16 | Firsocostat+NacCar | | | -2.951 | | -2.058 | | -0.893 |
| 17 | Cilofexor+LIRQ[2.5]S[10]; NacCar | | | -1.036 | | -0.974 | | -0.062 |
| 18 | Cilofexor+LIRQ[2.5]S[10]; NacCar | | | -1.123 | | -0.863 | | -0.260 |
| 19 | Cilofexor+LIRQ[2.5]S[10]; NacCar | | | -0.935 | | -0.776 | | -0.159 |
| 20 | Cilofexor+LIRQ[2.5]S[10]; NacCar | | | -1.142 | | -0.784 | | -0.357 |
| 21 | Cilofexor+NacCar | | | -1.672 | | -1.710 | | 0.038 |
| 22 | Cilofexor+NacCar | | | -1.460 | | -1.599 | | 0.139 |
| 23 | Cilofexor+NacCar | | | -1.466 | | -1.512 | | 0.046 |
| 24 | Cilofexor+NacCar | | | -1.606 | | -1.520 | | -0.085 |

### MCP1/CCL2 Secretion

Table E6 shows the level of MCP1/CCL2 secretion subtracted from the baseline (sFA+TNF-α) and normalized to nuclei in primary human hepatocytes cells from one healthy donor (Donor 2).

Treatment with LIRQ[2.5]S[10]NacCar or NacCar highly reduced MCP1 level compared to the baseline (Table E6, column 5). Treatment with Firsocostat or Selonsertib shows no change or a slight reduction in MCP1 secretion level compared to the baseline. Treatment with Cilofexor shows no change or a slight increase in MCP1 secretion level (Table E6, column 8). Similar to lipid, reducing MCP1 is the desired phenotype. Thus, a negative synergy score corresponds to a stronger reduction of MCP1 in the Rx-AA composition combination compared to the individual treatments.

Treatment with either Firsocostat, Selonsertib or Cilofexor in combination with NacCar show a negative synergy score suggesting that the combination Rx-NacCar reduces MCP1 at a higher level than the Rx (Firsocostat, Selonsertib, Cilofexor) or NacCar alone. Treatment with the same Rx in combination with LIRQ[2.5]S[10]NacCar show a negative synergy score with Firsocostat at two doses tested and with Cilofexor at three doses tested.

TABLE E6

| Row | Donor | phenotype | Amino Acid | AA Conc. (X)* | AA+Supp. effect | Rx | Rx Conc. (µM) | Rx effect |
|---|---|---|---|---|---|---|---|---|
| | | | Supplement (Supp.) | Nac/Car (mM) | | | | |
| 1 | 2 | MCP1 | LIRQ[2.5]S[10]; NacCar | 20X; 5 | -0.536 | Selonsertib | 0.0625 | 0.004 |
| 2 | 2 | MCP1 | LIRQ[2.5]S[10]; NacCar | 20X; 5 | -0.536 | Selonsertib | 0.25 | -0.350 |
| 3 | 2 | MCP1 | LIRQ[2.5]S[10]; NacCar | 20X; 5 | -0.536 | Selonsertib | 1 | -0.411 |
| 4 | 2 | MCP1 | LIRQ[2.5]S[10]; NacCar | 20X; 5 | -0.536 | Selonsertib | 4 | -0.463 |
| 5 | 2 | MCP1 | NacCar-5 | 5 | -0.701 | Selonsertib | 0.0625 | 0.004 |
| 6 | 2 | MCP1 | NacCar-5 | 5 | -0.701 | Selonsertib | 0.25 | -0.350 |
| 7 | 2 | MCP1 | NacCar-5 | 5 | -0.701 | Selonsertib | 1 | -0.411 |
| 8 | 2 | MCP1 | NacCar-5 | 5 | -0.701 | Selonsertib | 4 | -0.463 |
| 9 | 2 | MCP1 | LIRQ[2.5]S[10]; NacCar | 20X; 5 | -0.536 | Firsocostat | 0.0625 | -0.147 |
| 10 | 2 | MCP1 | LIRQ[2.5]S[10]; NacCar | 20X; 5 | -0.536 | Firsocostat | 0.25 | -0.542 |
| 11 | 2 | MCP1 | LIRQ[2.5]S[10]; NacCar | 20X; 5 | -0.536 | Firsocostat | 1 | -0.682 |
| 12 | 2 | MCP1 | LIRQ[2.5]S[10]; NacCar | 20X; 5 | -0.536 | Firsocostat | 4 | -0.510 |
| 13 | 2 | MCP1 | NacCar-5 | 5 | -0.701 | Firsocostat | 0.0625 | -0.147 |
| 14 | 2 | MCP1 | NacCar-5 | 5 | -0.701 | Firsocostat | 0.25 | -0.542 |
| 15 | 2 | MCP1 | NacCar-5 | 5 | -0.701 | Firsocostat | 1 | -0.682 |
| 16 | 2 | MCP1 | NacCar-5 | 5 | -0.701 | Firsocostat | 4 | -0.510 |
| 17 | 2 | MCP1 | LIRQ[2.5]S[10]; NacCar | 20X; 5 | -0.536 | Cilofexor | 0.0313 | -0.265 |
| 18 | 2 | MCP1 | LIRQ[2.5]S[10]; NacCar | 20X; 5 | -0.536 | Cilofexor | 0.125 | 0.229 |
| 19 | 2 | MCP1 | LIRQ[2.5]S[10]; NacCar | 20X; 5 | -0.536 | Cilofexor | 0.5 | 0.525 |
| 20 | 2 | MCP1 | LIRQ[2.5]S[10]; NacCar | 20X; 5 | -0.536 | Cilofexor | 2 | 0.301 |
| 21 | 2 | MCP1 | NacCar-5 | 5 | -0.701 | Cilofexor | 0.0313 | -0.265 |
| 22 | 2 | MCP1 | NacCar-5 | 5 | -0.701 | Cilofexor | 0.125 | 0.229 |
| 23 | 2 | MCP1 | NacCar-5 | 5 | -0.701 | Cilofexor | 0.5 | 0.525 |
| 24 | 2 | MCP1 | NacCar-5 | 5 | -0.701 | Cilofexor | 2 | 0.301 |
| Row | Combination [AA+Supp.+Rx] | | | Combination effect | | Sum [AA+Supp.+Rx] effect | | Synergy Score (Combo. Effect — Sum [AA+Supp.+Rx] effect) |
| 1 | Selonsertib+LIRQ[2.5]S[10]; NacCar | | | -0.499 | | -0.532 | | 0.033 |
| 2 | Selonsertib+LIRQ[2.5]S[10]; NacCar | | | -0.542 | | -0.886 | | 0.344 |
| 3 | Selonsertib+LIRQ[2.5]S[10]; NacCar | | | -0.790 | | -0.947 | | 0.157 |
| 4 | Selonsertib+LIRQ[2.5]S[10]; NacCar | | | -0.917 | | -0.999 | | 0.081 |
| 5 | Selonsertib+NacCar | | | -1.344 | | -0.698 | | -0.647 |
| 6 | Selonsertib+NacCar | | | -1.377 | | -1.051 | | -0.326 |
| 7 | Selonsertib+NacCar | | | -1.088 | | -1.112 | | 0.025 |
| 8 | Selonsertib+NacCar | | | -1.523 | | -1.164 | | -0.359 |
| 9 | Firsocostat+LIRQ[2.5]S[10]; NacCar | | | -0.985 | | -0.683 | | -0.302 |
| 10 | Firsocostat+LIRQ[2.5]S[10]; NacCar | | | -0.968 | | -1.078 | | 0.110 |
| 11 | Firsocostat+LIRQ[2.5]S[10]; NacCar | | | -1.192 | | -1.218 | | 0.026 |
| 12 | Firsocostat+LIRQ[2.5]S[10]; NacCar | | | -1.767 | | -1.046 | | -0.721 |
| 13 | Firsocostat+NacCar | | | -1.206 | | -0.849 | | -0.357 |
| 14 | Firsocostat+NacCar | | | -1.509 | | -1.243 | | -0.266 |
| 15 | Firsocostat+NacCar | | | -1.940 | | -1.384 | | -0.557 |
| 16 | Firsocostat+NacCar | | | -2.173 | | -1.211 | | -0.962 |
| 17 | Cilofexor+LIRQ[2.5]S[10]; NacCar | | | -0.731 | | -0.801 | | 0.069 |
| 18 | Cilofexor+LIRQ[2.5]S[10]; NacCar | | | -0.971 | | -0.307 | | -0.663 |
| 19 | Cilofexor+LIRQ[2.5]S[10]; NacCar | | | -0.489 | | -0.011 | | -0.478 |
| 20 | Cilofexor+LIRQ[2.5]S[10]; NacCar | | | -0.466 | | -0.235 | | -0.230 |
| 21 | Cilofexor+NacCar | | | -0.936 | | -0.966 | | 0.030 |
| 22 | Cilofexor+NacCar | | | -0.713 | | -0.472 | | -0.240 |
| 23 | Cilofexor+NacCar | | | -0.539 | | -0.176 | | -0.363 |
| 24 | Cilofexor+NacCar | | | -0.584 | | -0.401 | | -0.184 |

### Example 2: Treatment of Hepatic Insufficiency Subjects with an Amino Acid Composition

In some embodiments, the amino acid compositions of the present invention (e.g., a composition comprising L-leucine, L-isoleucine, L-valine, L-lysine acetate, L-histidine, L-threonine, L-ornithine, L-aspartate, and one or both of N-acetylcysteine or L-carnitine) will improve structural, functional, biomarkers and outcomes compared to placebo and other treatments (e.g., a composition comprising L-leucine, L-isoleucine, L-valine, L-lysine acetate, L-histidine, L-threonine, L-ornithine, and L-aspartate). The compositions are administered to human subjects with hepatic insufficiency, for a suitable duration (e.g. for 2 weeks to 2 years). Structural assessments include measurements of muscle mass, muscle volume cross sectional area, muscle fiber cross sectional area, midarm circumference, skeletal muscle index (e.g, height normalized cross sectional area of L3 abdominal muscle cm²/m²), lean body mass, muscle fat, myokines, portal hypertension, liver fibrosis (e.g., fibroscan), glucose tolerance, insulin sensitivity, cytokines/chemokines, and gut microbiota content or metabolism.

In some embodiments, the amino acid compositions described herein will improve (in some cases better than previous treatments) markers of physical function including MELD or Child’s Pugh score, liver frailty index, composite measures grip strength, balance, chair stand performance, short performance physical battery, exercise endurance, 6-minute walk distance, number connection tests (or equivalent minimal HE neuropsychometric assessment such as Stroop or PHES), West Haven Criteria for Overt HE, or similar assessment. In some embodiments, the amino acid compositions of the present invention will improve biomarkers including but not limited to albumin, IGF-1, GDF family members, Myostatin and other TGFb family signals, ammonia, plasma amino acid profile, inflammatory cytokines like TNF, IL-6, and liver function tests (e.g., AST, ALT), and markers of fibrosis (Pro-C3). In some embodiments, the amino acid compositions of the present invention will improve outcomes for subjects living with liver disease as measured by patient reports (e.g., Chronic Liver Disease Questionnaire), hospitalization frequency or duration, decompensation frequency, bouts of hepatic encephalopathy or recurrence between events, infection frequency and duration, or progression to hepatocellular carcinoma.

### Example 3: Bile Duct Ligation Experiments

Bile duct ligated (BDL) rats are used to model effects of the amino acid compositions on pharmacokinetic and pharmacodynamic properties, including but not limited to markers of amino acid homeostasis, disease pathophysiology, disease histology, and functional consequences, in animals with liver cirrhosis.

Relevance: End-stage liver disease results in a complex pathophysiology arising from liver failure that has systemic consequences across all organs. As the liver is a critical organ for maintaining amino acid homeostasis, liver failure has profound dysregulation of plasma amino acid concentrations which are associated with disease severity and mortality. As the largest reservoir of protein in the body, skeletal muscle is a critical source of amino acids and profound wasting is observed in patients with liver cirrhosis, and skeletal muscle mass predicts mortality in cirrhotic patients. Bile duct ligation is a well-accepted model of cholestasis-induced liver disease that manifests with hyperammonemia and dysregulation of plasma amino acids. Muscle mass and function worsen over time in the BDL rat. As such, the BDL rat is a useful pre-clinical model to understand the complex pathophysiology arising from liver failure and examine consequences of various interventions on multi-systemic effects and markers of disease

Methods: 6-12 week old rats are subjected to a surgical procedure where a section of the bile duct is isolated, ligated, and cauterized. Starting one to two weeks post-surgery, the animals are treated twice daily by oral gavage with amino acid compositions including the amino acid compositions of the present invention (e.g., amino acid composition treatments) for up to one month.

Pharmacokinetic properties of the amino acid compositions are assessed in both the fed and fasted state at the beginning, middle, and end of the treatment period. Pharmacokinetic analysis is performed by collecting blood from the jugular vein in heparin tubes prior to and 0.25 hr, 0.5 hr, 1 hr, 1.5 hr, 2 hr, 3 hr, and 4 hr after administration of the amino acid composition. Plasma amino acid concentrations are assessed by LC-MS or an equivalent method. In addition, plasma is analyzed for ammonia levels, cytokine and chemokine levels (e.g., TNF, IL-6, etc.), markers of liver damage (e.g. ALT, AST), protein (e.g. Total, albumin, etc), ammonia levels, or markers of muscle wasting (e.g., methylated-histidine or myostatin).

Muscle function is measured by assessing forelimb and hindlimb grip strength using a standard meter and testing at the beginning, middle, and end of the treatment period.

Alternatively, muscle function is assessed by rotorod performance, treadmill endurance, or plantarflexor torque or similar neuromuscular assays. At the end of the study, hindlimb muscles are collected, weighed, and embedded in OCT freezing medium. Thin cryosections are prepared and immunostained or stained with hematoxylin and eosin (H&E), nile red, oil red O, or myosin isoforms and muscle mass is assessed by quantifying the cross-sectional area of myofibers of each muscle in the section.

Effects on liver function are assessed by collecting the entire liver at the end of the study, weighing it, and preparing it for paraffin embedding by fixing specific lobes in 10% formalin overnight. H&E staining, staining for fibrosis (Sirius Red), intrahepatic lipid accumulation (oil red O staining), markers of inflammation, and other standard measures and routine assessments are made.

Effects on amino acid homeostasis are assessed using targeted metabolite profiling and untargeted metabolomics of plasma, liver and muscle. Plasma collections at the beginning, middle, and end of the study are used to measure disease progression and tissue analysis is used to determine further consequences of treatment with various amino acid compositions.

### Example 4: Hepatic Albumin, Fat Accumulation, and Inflammation in Cirrhosis-Induced Hypoalbuminemia

Media comprising amino acids consistent with profiles of cirrhotic individuals is tested for effect on production of albumin in vitro. In some instances, assay and culture conditions include TNFα, free saturated fatty acids, or both. Effects of the amino acid compositions described herein, constituents and comparator compositions on albumin production, lipid accumulation, mitochondrial/metabolic function are determined.

Relevance: Plasma amino acid concentrations are disrupted in patients with liver cirrhosis and predict mortality in end stage liver disease (Kinny-Koster et al., 2016). Plasma albumin levels are an important metric in Child’s-Pugh scoring of liver disease severity and malnutrition which results in hypoalbuminemia is a significant complication of liver cirrhosis (Loza, 2014). BCAAs (specifically LIVact) has been approved outside the United States for the treatment of hypoalbuminemia in liver cirrhosis. TNF is an inflammatory signal that induces acute phase reaction and downregulation of negative phase proteins like albumin. In addition, presence of free fatty acids induces deregulation of lipid metabolism, inflammatory and metabolic signaling derangements.

Methods: In some embodiments, the amino acid compositions of the present invention will increase hepatocyte albumin production (e.g., as assessed using the C3A derivative clone of the HepG2 Hepatocellular Carcinoma cell line (ATCC, CRL-10741), lipid accumulation (e.g. as assessed using oil red O, nile red stain, or equivalent), or metabolic function (e.g., as assessed using Seahorse metabolic assays)). Cells are seeded in Dulbecco’s Modified Eagle Medium (DMEM, Corning), or appropriate background media, supplemented with fetal bovine serum (Corning) and 0.2% Primocin (InVivoGen, San Diego, CA) and incubated at 37° C., 5% CO₂. On day 1, the cell medium is replaced with amino acid free DMEM (US Biologicals, Salem, MA) supplemented with 0.1% heat inactivated fetal bovine serum (HI-FBS, HyClone), 100 ug/mL Primocin (InVivoGen), amino acids supplemented at 0.1X, 0.25X, 0.5X, 1X or higher concentrations relative to plasma levels consistent with rations observed in cirrhotic individuals (Kakazu et al., 2013) and a dose curve of defined amino acid compositions at 5X, 10X, 20X and 40X of basal media concentrations is added to the cells. Cells are cultured for 48 hours at 37° C., 5% CO₂, media is collected, cells are washed 1x in PBS, fixed in 4% paraformaldehyde, washed 2x in PBS, nuclei are stained with Hoechst 33342 according to manufacturer’s instructions (Life Technologies, H3570) and then washed 2x in PBS. Media albumin levels are assessed by R&D Systems’s DuoSet ELISA Development System for Human Serum Albumin (R&D Systems, DY1455) or equivalent assays and nuclei are counted using Molecular Devices Image Express High Content Screening platform and pre-installed nuclei counting analysis pipeline. Albumin levels are normalized to total nuclei in order to derive a per cell albumin production ratio. Stained intracellular lipids are anaylzed according to manufacturers instructions for specific stains. Seahorse experiments are performed according to manufacturers recommendations and measurements will include total ATP production rate, glycolytic ATP production rate, and mitochondrial ATP production rate using a commercially available kit (Agilent Seahorse XF Real-Time ATP Rate Assay Kit) according to manufacturer-supplied protocol on a Seahorse XFe instrument.

In some embodiments, the amino acid compositions of the present invention will have similar or improved activity at lower concentrations than other treatments (e.g., a composition comprising L-leucine, L-isoleucine, L-valine, L-lysine acetate, L-histidine, L-threonine, L-ornithine, and L-aspartate) in a model of hepatic cirrhosis that includes albumin synthesis, hepatic lipid and mitochondrial metabolism.

### Example 5: Lipid Accumulation and Protein Synthesis in Ammonia and/or TNF-Induced Myotube Atrophy and/or defects in Myotube Fusion Indices

Relevance: Patients with liver cirrhosis and concomitant muscle wasting (Cirrhotic Sarcopenia, CS) are especially susceptible to co-morbidities and complications associated with end stage liver disease. Muscle wasting in cirrhosis is multifactorial and complex but driven by inflammation, altered plasma amino acid availability, hyperammonemia, and myostatin expression. Protein synthesis is important for muscle mass accretion while muscle fat accumulation has negative consequences for insulin sensitivity, glucose disposal, and anabolic sensitivity. Ammonia and TNFα promote myostatin expression and inhibit protein synthesis, both of which are important pathologies CS (Qiu et al., 2013).

Methods: Primary human myoblasts are differentiated to form myotubes (multinucleated syncytia) that which share many features with human muscle tissue. Relevant phenotypic readouts of myotube cultures include: area, nucleii, fusion index, lipid accumulation, catabolic signaling, anabolic resistance and myogenesis. In one assay, on Day 0 expanded primary myoblasts from a healthy human donor are seeded at 10,000 cells per well for 24 hour at 37deg C. On Day 1, cells are incubated in differentiation media based on DMEM comprising 0.1% horse serum and containing indicated amino acid combinations at 4X, 10X, 20X, and 30X of the concentration in the cirrhosis media or with IGF-1 (150 ng/mL) as a positive control. Importantly, treatment groups are prepared in PBS, and final concentration in all wells, including controls, is 10% PBS. On Day 2, cells are switched to Cirrhosis Media based on DMEM comprising 0.1% horse serum and 0.5X amino acids at ratios of plasma of cirrhosis (Kakazu et al., 2013) containing atrophy inducer (TNFalpha or ammonia acetate) and again treated according to Day 1. On Day 6, cells are fixed with paraformaldehyde for up to 30 minutes at room temperature. Cells may be co-incubated with 0, 200, or 400 µM Free Fatty Acids, e.g., 2:1 oleate to palmitate. Fixed cells are processed for immunostaining with primary antibody against Troponin T to stain myotubes and Hoeschst dye for nuclei, nile red for intracellular lipid content. Image acquisition is performed at 10x magnification and analysis using a proprietary and dedicated algorithm on the Acapella High Content Imaging System (Perkin Elmer, CYTOO). As an alternative protocol of this assay, myotubes are first differentiated and established for up to 5 days and then treated with atrophy inducers (TNFalpha or ammonia acetate) and the specific amino acid compositions described herein.

In some embodiments, the amino acid compositions of the present invention will have similar or improved activity at lower concentrations than other treatments (e.g., a composition comprising L-leucine, L-isoleucine, L-valine, L-lysine acetate, L-histidine, L-threonine, L-ornithine, and L-aspartate) in a model of muscle culture that includes cell size, protein synthesis, myogenesis, and lipid accumulation.

### Summary of Examples

Liver diseases such as NASH or NAFLD are complex and driven by a multitude of intersecting pathways. Maintaining liver health and function requires coordination of many biological, cellular and molecular processes. As shown in the Examples herein, the amino acid compositions disclosed in this application (e.g., compositions comprising NacCar, or LIRNacCar or LRQNacCar, such as LIRQNacCarS) are able to synergize when combined with a therapeutic agent as described herein to reduce hepatocyte lipid and inflammation. A combination of Nac and Car (“NacCar”) is also able to reduce hepatocyte lipid and inflammation with or without further combination with a therapeutic agent as disclosed herein. Thus, these combinations and compositions exhibit unexpected synergistic effects and utility in the treatment of NASH, NAFLD, hepatic lipid accumulation, and/or hepatic inflammation.

In addition, liver diseases such as cirrhosis and/or hepatic encephalopathy and related symptoms are also complex and multifactorial. As shown in the Examples above, the amino acid compositions disclosed in this application (e.g., compositions comprising NacCar, or LIVODHKTNacCar) are effective to reduce inflammation, improve (e.g., reduce) lipid levels in muscle and liver, and improved muscle mass, quality and/or function. These compositions exhibit unexpected synergistic effects and utility in the treatment of cirrhosis and/or hepatic encephalopathy and symptoms related thereto.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

What is claimed is:

1. A method for treating a symptom chosen from one, two, three, four, five, six, seven, eight, nine, ten, or more (e.g., all) of: decreased fat metabolism, hepatocyte apoptosis, hepatocyte ballooning, inflammation of adipose tissue, inflammation of hepatic tissue, fibrosis, liver injury, steatosis, glucose tolerance, insulin resistance, or oxidative stress, comprising administering to a subject in need thereof an effective amount of a combination (e.g., one or more compositions or dosage forms) comprising a composition and a therapeutic agent, wherein the composition comprises:
(a) N-acetylcysteine (NAC) or a salt thereof, or a dipeptide or a salt thereof, comprising NAC;
(b) L-carnitine or a salt thereof, or a dipeptide or a salt thereof, comprising L-carnitine; or acetyl-L-carnitine (ALCAR) or a salt thereof or a dipeptide or salt thereof, or a tripeptide or salt thereof, comprising ALCAR; and
the therapeutic agent is chosen from one or both of the following categories:
(i) a hepatic fat accumulation inhibitor; or
(ii) one or both of an oxidative stress or hepatic inflammation inhibitor, thereby treating the symptom in the subject.

2. A method for treating a liver disease or disorder, comprising administering to a subject in need thereof an effective amount of a combination (e.g., one or more compositions or dosage forms) comprising a composition and a therapeutic agent, wherein the composition comprises:
(a) N-acetylcysteine (NAC) or a salt thereof, or a dipeptide or a salt thereof, comprising NAC;
(b) L-carnitine or a salt thereof, or a dipeptide or a salt thereof, comprising L-carnitine; or acetyl-L-carnitine (ALCAR) or a salt thereof or a dipeptide or salt thereof, or a tripeptide or salt thereof, comprising ALCAR; and
the therapeutic agent is chosen from one or both of the following categories:
(i) a hepatic fat accumulation inhibitor; or
(ii) one or both of an oxidative stress or hepatic inflammation inhibitor, thereby treating the liver disease or disorder in the subject.

3. A method for improving liver function, comprising administering to a subject in need thereof an effective amount of a combination (e.g., one or more compositions or dosage forms) comprising a composition and a therapeutic agent, wherein the composition comprises:
(a) N-acetylcysteine (NAC) or a salt thereof, or a dipeptide or a salt thereof, comprising NAC;
(b) L-carnitine or a salt thereof, or a dipeptide or a salt thereof, comprising L-carnitine; or acetyl-L-carnitine (ALCAR) or a salt thereof or a dipeptide or salt thereof, or a tripeptide or salt thereof, comprising ALCAR; and
the therapeutic agent is chosen from one or both of the following categories:
(i) a hepatic fat accumulation inhibitor; or
(ii) one or both of an oxidative stress or hepatic inflammation inhibitor, thereby improving liver function in the subject.

4. A method for treating a symptom chosen from one, two, three, four, five, six, seven, eight, nine, ten, or more (e.g., all) of: decreased fat metabolism, hepatocyte apoptosis, hepatocyte ballooning, inflammation of adipose tissue, inflammation of hepatic tissue, fibrosis, liver injury, steatosis, glucose tolerance, insulin resistance, or oxidative stress, comprising administering to a subject in need thereof an effective amount of a combination (e.g., one or more compositions or dosage forms) comprising a composition and a therapeutic agent, wherein the composition comprises:
a) a leucine amino acid entity chosen from:
i) L-leucine or a salt thereof,
ii) a dipeptide or salt thereof, or tripeptide or salt thereof, comprising L-leucine, or
iii) β-hydroxy-β-methylbutyrate (HMB) or a salt thereof;
b) a arginine amino acid entity chosen from:
i) L-arginine or a salt thereof,
ii) a dipeptide or salt thereof, or tripeptide or salt thereof, comprising L-arginine,
iii) ornithine or a salt thereof,
iv) a dipeptide or salt thereof, or tripeptide or salt thereof, comprising ornithine,
v) creatine or a salt thereof, or
vi) a dipeptide or salt thereof, or tripeptide or salt thereof, comprising creatine;
c) L-glutamine or a salt thereof or a dipeptide or salt thereof, or tripeptide or salt thereof, comprising L-glutamine;
d) N-acetylcysteine (NAC) or a salt thereof or a dipeptide or salt thereof, comprising NAC;
e) one or both of
i) L-serine or a salt thereof or a dipeptide or salt thereof, or tripeptide or salt thereof, comprising L-serine; or
ii) L-carnitine or a salt thereof or a dipeptide or salt thereof, or tripeptide or salt thereof, comprising L-carnitine; and the therapeutic agent is chosen from one or both of the following categories:
(i) a hepatic fat accumulation inhibitor; or
(ii) one or both of an oxidative stress or hepatic inflammation inhibitor, wherein optionally one or both of:
the wt. % of the serine amino acid entity is at least 8 wt. % of the total amino acid entity components or total components in the composition; or
the wt. % of the carnitine entity is at least 2 wt. % of the total amino acid entity components or total components in the composition,
thereby treating the symptom in the subject.

5. A method for treating a liver disease or disorder, comprising administering to a subject in need thereof an effective amount of a combination (e.g., one or more compositions or dosage forms) comprising a composition and a therapeutic agent, wherein the composition comprises:
a) a leucine amino acid entity chosen from:
i) L-leucine or a salt thereof,
ii) a dipeptide or salt thereof, or tripeptide or salt thereof, comprising L-leucine, or
iii) β-hydroxy-β-methylbutyrate (HMB) or a salt thereof;
b) a arginine amino acid entity chosen from:
i) L-arginine or a salt thereof,
ii) a dipeptide or salt thereof, or tripeptide or salt thereof, comprising L-arginine,
iii) ornithine or a salt thereof,
iv) a dipeptide or salt thereof, or tripeptide or salt thereof, comprising ornithine,
v) creatine or a salt thereof, or
vi) a dipeptide or salt thereof, or tripeptide or salt thereof, comprising creatine;
c) L-glutamine or a salt thereof or a dipeptide or salt thereof, or tripeptide or salt thereof, comprising L-glutamine;
d) N-acetylcysteine (NAC) or a salt thereof or a dipeptide or salt thereof, comprising NAC;
e) one or both of
i) L-serine or a salt thereof or a dipeptide or salt thereof, or tripeptide or salt thereof, comprising L-serine; or
ii) L-carnitine or a salt thereof or a dipeptide or salt thereof, or tripeptide or salt thereof, comprising L-carnitine; and
the therapeutic agent is chosen from one or both of the following categories:
(i) a hepatic fat accumulation inhibitor; or
(ii) one or both of an oxidative stress or hepatic inflammation inhibitor, wherein optionally one or both of:
the wt. % of the serine amino acid entity is at least 8 wt. % of the total amino acid entity components or total components in the composition; or
the wt. % of the carnitine entity is at least 2 wt. % of the total amino acid entity components or total components in the composition,
thereby treating the liver disease or disorder in the subject.

6. A method for improving liver function, comprising administering to a subject in need thereof an effective amount of a combination (e.g., one or more compositions or dosage forms) comprising a composition and a therapeutic agent, wherein the composition comprises:
a) a leucine amino acid entity chosen from:
i) L-leucine or a salt thereof,
ii) a dipeptide or salt thereof, or tripeptide or salt thereof, comprising L-leucine, or
iii) β-hydroxy-β-methylbutyrate (HMB) or a salt thereof;
b) a arginine amino acid entity chosen from:
i) L-arginine or a salt thereof,
ii) a dipeptide or salt thereof, or tripeptide or salt thereof, comprising L-arginine,
iii) ornithine or a salt thereof,
iv) a dipeptide or salt thereof, or tripeptide or salt thereof, comprising ornithine,
v) creatine or a salt thereof, or
vi) a dipeptide or salt thereof, or tripeptide or salt thereof, comprising creatine;
c) L-glutamine or a salt thereof or a dipeptide or salt thereof, or tripeptide or salt thereof, comprising L-glutamine;
d) N-acetylcysteine (NAC) or a salt thereof or a dipeptide or salt thereof, comprising NAC;
e) one or both of
i) L-serine or a salt thereof or a dipeptide or salt thereof, or tripeptide or salt thereof, comprising L-serine; or
ii) L-carnitine or a salt thereof or a dipeptide or salt thereof, or tripeptide or salt thereof, comprising L-carnitine; and
the therapeutic agent is chosen from one or both of the following categories:
(i) a hepatic fat accumulation inhibitor; or
(ii) one or both of an oxidative stress or hepatic inflammation inhibitor, wherein optionally one or both of:
the wt. % of the serine amino acid entity is at least 8 wt. % of the total amino acid entity components or total components in the composition; or
the wt. % of the carnitine entity is at least 2 wt. % of the total amino acid entity components or total components in the composition,
thereby improving liver function in the subject.

7. The method of any of claims 4-6, wherein the composition further comprises: f) L-isoleucine or a salt thereof or a dipeptide or salt thereof, or a tripeptide or salt thereof, comprising L-isoleucine.

8. The method of any of claims 1-7, wherein the composition comprises:
a) the leucine amino acid entity is L-leucine or a salt thereof;
b) the arginine amino acid entity is L-arginine or a salt thereof;
c) L-glutamine or a salt thereof;
d) NAC or a salt thereof;
e) L-serine or a salt thereof
f) L-carnitine or a salt thereof; and
g) L-isoleucine or a salt thereof.

9. A method for treating a symptom chosen from one, two, three, four, five, six, seven, eight, nine, ten, or more (e.g., all) of: decreased fat metabolism, hepatocyte apoptosis, hepatocyte ballooning, inflammation of adipose tissue, inflammation of hepatic tissue, fibrosis, liver injury, steatosis, glucose tolerance, insulin resistance, or oxidative stress, comprising administering to a subject in need thereof an effective amount of combination (e.g., one or more compositions or dosage forms) comprising a composition and a therapeutic agent, wherein the composition comprises:
a) a leucine amino acid entity,
b) an isoleucine amino acid entity,
c) a arginine amino acid entity,
d) a N-acetylcysteine (NAC) entity; and
e) a carnitine entity; and
the therapeutic agent is chosen from one or both of the following categories:
(i) a hepatic fat accumulation inhibitor; or
(ii) one or both of an oxidative stress or hepatic inflammation inhibitor,
wherein the total wt. % of (a)-(e) is greater than the total wt. % of other amino acid entities in the composition (e.g., in dry form); and
wherein optionally the wt. % of the carnitine entity is at least 2 wt. % of the amino acid entites or the total components in the composition (e.g., in dry form),
thereby treating the symptom in the subject.

10. A method for treating a liver disease or disorder, comprising administering to a subject in need thereof an effective amount of combination (e.g., one or more compositions or dosage forms) comprising a composition and a therapeutic agent, wherein the composition comprises:
a) a leucine amino acid entity,
b) an isoleucine amino acid entity,
c) a arginine amino acid entity,
d) a N-acetylcysteine (NAC) entity; and
e) a carnitine entity; and
the therapeutic agent is chosen from one or both of the following categories:
(i) a hepatic fat accumulation inhibitor; or
(ii) one or both of an oxidative stress or hepatic inflammation inhibitor,
wherein the total wt. % of (a)-(e) is greater than the total wt. % of other amino acid entities in the composition (e.g., in dry form); and
wherein optionally the wt. % of the carnitine entity is at least 2 wt. % of the amino acid entites or the total components in the composition (e.g., in dry form),
thereby treating the liver disease or disorder in the subject.

11. A method for improving liver function, comprising administering to a subject in need thereof an effective amount of combination (e.g., one or more compositions or dosage forms) comprising a composition and a therapeutic agent, wherein the composition comprises:
a) a leucine amino acid entity,
b) an isoleucine amino acid entity,
c) a arginine amino acid entity,
d) a N-acetylcysteine (NAC) entity; and
e) a carnitine entity; and
the therapeutic agent is chosen from one or both of the following categories:
(i) a hepatic fat accumulation inhibitor; or
(ii) one or both of an oxidative stress or hepatic inflammation inhibitor,
wherein the total wt. % of (a)-(e) is greater than the total wt. % of other amino acid entities in the composition (e.g., in dry form); and
wherein optionally the wt. % of the carnitine entity is at least 2 wt. % of the amino acid entites or the total components in the composition (e.g., in dry form), thereby improving liver function in the subject.

12. A combination (e.g., one or more compositions or dosage forms) comprising a composition and a therapeutic agent, wherein the composition comprises:
(a) N-acetylcysteine (NAC) or a salt thereof, or a dipeptide or a salt thereof, comprising NAC;
(b) L-carnitine or a salt thereof, or a dipeptide or a salt thereof, comprising L-carnitine; or acetyl-L-carnitine (ALCAR) or a salt thereof or a dipeptide or salt thereof, or a tripeptide or salt thereof, comprising ALCAR; and
the therapeutic agent is chosen from one or both of the following categories:
(i) a hepatic fat accumulation inhibitor; or
(ii) one or both of an oxidative stress or hepatic inflammation inhibitor.

13. A combination (e.g., one or more compositions or dosage forms) comprising a composition and a therapeutic agent, wherein the composition comprises:
a) a leucine amino acid entity chosen from:
i) L-leucine or a salt thereof,
ii) a dipeptide or salt thereof, or tripeptide or salt thereof, comprising L-leucine, or
iii) β-hydroxy-β-methylbutyrate (HMB) or a salt thereof;
b) a arginine amino acid entity chosen from:
i) L-arginine or a salt thereof,
ii) a dipeptide or salt thereof, or tripeptide or salt thereof, comprising L-arginine,
iii) ornithine or a salt thereof,
iv) a dipeptide or salt thereof, or tripeptide or salt thereof, comprising ornithine,
v) creatine or a salt thereof, or
vi) a dipeptide or salt thereof, or tripeptide or salt thereof, comprising creatine;
c) L-glutamine or a salt thereof or a dipeptide or salt thereof, or tripeptide or salt thereof, comprising L-glutamine;
d) N-acetylcysteine (NAC) or a salt thereof or a dipeptide or salt thereof, comprising NAC;
e) one or both of
i) L-serine or a salt thereof or a dipeptide or salt thereof, or tripeptide or salt thereof, comprising L-serine; or
ii) L-carnitine or a salt thereof or a dipeptide or salt thereof, or tripeptide or salt thereof, comprising L-carnitine; and
the therapeutic agent is chosen from one or both of the following categories:
(i) a hepatic fat accumulation inhibitor; or
(ii) one or both of an oxidative stress or hepatic inflammation inhibitor, wherein optionally one or both of:
the wt. % of the serine amino acid entity is at least 8 wt. % of the total amino acid entity components or total components in the composition; or
the wt. % of the carnitine entity is at least 2 wt. % of the total amino acid entity components or total components in the composition.

14. A combination (e.g., one or more compositions or dosage forms) comprising a composition and a therapeutic agent, wherein the composition comprises:
a) a leucine amino acid entity,
b) an isoleucine amino acid entity,
c) a arginine amino acid entity,
d) a N-acetylcysteine (NAC) entity; and
e) a carnitine entity; and
the therapeutic agent is chosen from one or both of the following categories:
(i) a hepatic fat accumulation inhibitor; or
(ii) one or both of an oxidative stress or hepatic inflammation inhibitor, wherein the total wt. % of (a)-(e) is greater than the total wt. % of other amino acid entities in the composition (e.g., in dry form); and
wherein optionally the wt. % of the carnitine entity is at least 2 wt. % of the amino acid entites or the total components in the composition (e.g., in dry form).

15. The combination of claim 14, wherein the composition further comprises: (f) one or both of a glutamine amino acid entity or a serine amino acid entity.

16. The method or combination of any preceding claim, wherein the therapeutic agent is a hepatic fat accumulation inhibitor.

17. The method or combination of any preceding claim, wherein the therapeutic agent is one or both of an oxidative stress or hepatic inflammation inhibitor.

18. The method or combination of any preceding claim, wherein the hepatic fat accumulation inhibitor is chosen from an acetyl-CoA carboxylase inhibitor, an FXR agonist, or a combination thereof.

19. The method or combination of claim 18, wherein the acetyl-CoA carboxylase inhibitor is chosen from GS-0976 (e.g., Firsocostat), PF-05221304, gemcabene, or a salt or a derivative of any of the foregoing.

20. The method or combination of either of claims 18 or 19, wherein the FXR agonist is chosen from GS-9674 (e.g., Cilofexor), LJN-452 (Tropifexor), obeticholic acid (OCA, e.g., Ocaliva), EDP-305, EYP001, or a salt or a derivative of any of the foregoing.

21. The method or combination of any preceding claim, wherein the one or both of an oxidative stress or hepatic inflammation inhibitor is an ASK1 inhibitor.

22. The method or combination of claim 21, wherein the ASK1 inhibitor is selonsertib or a salt or a derivative thereof.

23. A method of improving one or more of liver function, hyperammonemia, muscle mass, or muscle function, the method comprises administering to a subject with cirrhosis an effective amount of a composition comprising:
(a) N-acetylcysteine (NAC) or a salt thereof, or a dipeptide or a salt thereof, comprising NAC; and
(b) L-carnitine or a salt thereof, or a dipeptide or a salt thereof, comprising L-carnitine; or acetyl-L-carnitine (ALCAR) or a salt thereof or a dipeptide or salt thereof, or a tripeptide or salt thereof, comprising ALCAR,
wherein the total weight (wt.) % of (a) and (b) is greater than 50% of the total wt. of amino acid entities, or
wherein the wt. % of (b) is at least 2% and up to 10% of the total wt. of amino acid entities,
thereby improving or treating the symptom in the subject.

24. A method of improving or treating a symptom selected from the group consisting of hyperammonemia, ascites or complications associated with ascites, variceal bleeding, infection, hepatic encephalopathy, ammonia toxicity, hepatic insufficiency, decreased urea synthesis, inflammation of hepatic tissue, fibrosis, cirrhosis, muscle wasting, muscle catabolism, muscle atrophy, hypoalbuminemia, malnutrition, frailty, and coagulopathy, comprising administering to a subject in need thereof an effective amount of a composition comprising:
(a) N-acetylcysteine (NAC) or a salt thereof, or a dipeptide or a salt thereof, comprising NAC; and
(b) L-carnitine or a salt thereof, or a dipeptide or a salt thereof, comprising L-carnitine; or acetyl-L-carnitine (ALCAR) or a salt thereof or a dipeptide or salt thereof, or a tripeptide or salt thereof, comprising ALCAR,
wherein the total weight (wt.) % of (a) and (b) is greater than 50% of the total wt. of amino acid entities, or
wherein the wt. % of (b) is at least 2% and up to 10% of the total wt. of amino acid entities,
thereby improving or treating the symptom in the subject.

25. A method of treating or preventing a liver disease or disorder with one or both of hyperammonemia or muscle wasting, wherein the method comprises administering to a subject in need thereof an effective amount of a composition comprising:
(a) N-acetylcysteine (NAC) or a salt thereof, or a dipeptide or a salt thereof, comprising NAC; and
(b) L-carnitine or a salt thereof, or a dipeptide or a salt thereof, comprising L-carnitine; or acetyl-L-carnitine (ALCAR) or a salt thereof or a dipeptide or salt thereof, or a tripeptide or salt thereof, comprising ALCAR,
wherein the total weight (wt.) % of (a) and (b) is greater than 50% of the total wt. of amino acid entities, or
wherein the wt. % of (b) is at least 2% and up to 10% of the total wt. of amino acid entities,
thereby treating or preventing the liver disease or disorder.

26. A method of improving one or more of liver function, hyperammonemia, muscle mass, or muscle function, the method comprises administering to a subject with cirrhosis an effective amount of a composition comprising:
a) a Branched Chain Amino Acid (BCAA) entity chosen from a leucine amino acid entity, an isoleucine amino acid entity, a valine amino acid entity, or a combination of two or three BCAA entities;
b) a Urea Cycle Amino Acid (UCAA) entity chosen from an ornithine amino acid entity chosen from L-ornithine, ornithine α-ketoglutarate, ornithine HCl, citrulline, or a combination thereof; an aspartate amino acid entity; or a combination of two UCAA entities; and
c) an essential amino acid (EAA) entity chosen from a histidine amino acid entity, a lysine amino acid entity, or a threonine amino acid entity or a combination of two or three EAA entities; and
d) one or two of:
i) N-acetylcysteine (NAC) or a salt thereof, or a dipeptide or a salt thereof, comprising NAC; or
ii) L-carnitine or a salt thereof, or a dipeptide or a salt thereof, comprising L-carnitine; or acetyl-L-carnitine (ALCAR) or a salt thereof or a dipeptide or salt thereof, or a tripeptide or salt thereof, comprising ALCAR;
wherein:
i) at least one amino acid entity of (a)-(c) is not provided as a peptide of more than 20 amino acid residues in length;
ii) the total weight (wt.) % of (a)-(d) is greater than the total wt. % of non-amino acid entity protein components or other amino acid entity components in the composition on a dry weight basis; and
iii) two or more (e.g., all) of phenylalanine, tyrosine, or glutamine is absent from the composition, or if present, is present at less than 1 wt. % of the total wt. of the composition on a dry weight basis,
thereby improving or treating the symptom in the subject.

27. A method of improving or treating a symptom selected from the group consisting of hyperammonemia, ascites or complications associated with ascites, variceal bleeding, infection, hepatic encephalopathy, ammonia toxicity, hepatic insufficiency, decreased urea synthesis, inflammation of hepatic tissue, fibrosis, cirrhosis, muscle wasting, muscle catabolism, muscle atrophy, hypoalbuminemia, malnutrition, frailty, and coagulopathy, comprising administering to a subject in need thereof an effective amount of a composition comprising:
a) a Branched Chain Amino Acid (BCAA) entity chosen from a leucine amino acid entity, an isoleucine amino acid entity, a valine amino acid entity, or a combination of two or three BCAA entities;
b) a Urea Cycle Amino Acid (UCAA) entity chosen from an ornithine amino acid entity chosen from L-ornithine, ornithine α-ketoglutarate, ornithine HCl, citrulline, or a combination thereof; an aspartate amino acid entity; or a combination of two UCAA entities; and
c) an essential amino acid (EAA) entity chosen from a histidine amino acid entity, a lysine amino acid entity, or a threonine amino acid entity or a combination of two or three EAA entities; and
d) one or two of:
i) N-acetylcysteine (NAC) or a salt thereof, or a dipeptide or a salt thereof, comprising NAC; or
ii) L-carnitine or a salt thereof, or a dipeptide or a salt thereof, comprising L-carnitine; or acetyl-L-carnitine (ALCAR) or a salt thereof or a dipeptide or salt thereof, or a tripeptide or salt thereof, comprising ALCAR;
wherein:
i) at least one amino acid entity of (a)-(c) is not provided as a peptide of more than 20 amino acid residues in length;
ii) the total weight (wt.) % of (a)-(d) is greater than the total wt. % of non-amino acid entity protein components or other amino acid entity components in the composition on a dry weight basis; and
iii) two or more (e.g., all) of phenylalanine, tyrosine, or glutamine is absent from the composition, or if present, is present at less than 1 wt. % of the total wt. of the composition on a dry weight basis,
thereby improving or treating the symptom in the subject.

28. A method of treating or preventing a liver disease or disorder with one or both of hyperammonemia or muscle wasting, wherein the method comprises administering to a subject in need thereof an effective amount of a composition comprising:
a) a Branched Chain Amino Acid (BCAA) entity chosen from a leucine amino acid entity, an isoleucine amino acid entity, a valine amino acid entity, or a combination of two or three BCAA entities;
b) a Urea Cycle Amino Acid (UCAA) entity chosen from an ornithine amino acid entity chosen from L-ornithine, ornithine α-ketoglutarate, ornithine HCl, citrulline, or a combination thereof; an aspartate amino acid entity; or a combination of two UCAA entities; and
c) an essential amino acid (EAA) entity chosen from a histidine amino acid entity, a lysine amino acid entity, or a threonine amino acid entity or a combination of two or three EAA entities; and
d) one or two of:
i) N-acetylcysteine (NAC) or a salt thereof, or a dipeptide or a salt thereof, comprising NAC; or
ii) L-carnitine or a salt thereof, or a dipeptide or a salt thereof, comprising L-carnitine; or acetyl-L-carnitine (ALCAR) or a salt thereof or a dipeptide or salt thereof, or a tripeptide or salt thereof, comprising ALCAR;
wherein:
i) at least one amino acid entity of (a)-(c) is not provided as a peptide of more than 20 amino acid residues in length;
ii) the total weight (wt.) % of (a)-(d) is greater than the total wt. % of non-amino acid entity protein components or other amino acid entity components in the composition on a dry weight basis; and
iii) two or more (e.g., all) of phenylalanine, tyrosine, or glutamine is absent from the composition, or if present, is present at less than 1 wt. % of the total wt. of the composition on a dry weight basis,
thereby treating or preventing the liver disease or disorder.

29. A composition comprising:
(a) N-acetylcysteine (NAC) or a salt thereof, or a dipeptide or a salt thereof, comprising NAC; and
(b) L-carnitine or a salt thereof, or a dipeptide or a salt thereof, comprising L-carnitine; or acetyl-L-carnitine (ALCAR) or a salt thereof or a dipeptide or salt thereof, or a tripeptide or salt thereof, comprising ALCAR,
wherein the total weight (wt.) % of (a) and (b) is greater than 50% of the total wt. of amino acid entities or
wherein the wt. % of (b) is at least 2% and up to 10% of the total wt. of amino acid entities.

30. A composition comprising:
a) a Branched Chain Amino Acid (BCAA) entity chosen from a leucine amino acid entity, an isoleucine amino acid entity, a valine amino acid entity, or a combination of two or three BCAA entities;
b) a Urea Cycle Amino Acid (UCAA) entity chosen from an ornithine amino acid entity chosen from L-ornithine, ornithine α-ketoglutarate, ornithine HCl, citrulline, or a combination thereof; an aspartate amino acid entity; or a combination of two UCAA entities; and
c) an essential amino acid (EAA) entity chosen from a histidine amino acid entity, a lysine amino acid entity, or a threonine amino acid entity or a combination of two or three EAA entities; and
d) one or two of:
i) N-acetylcysteine (NAC) or a salt thereof, or a dipeptide or a salt thereof, comprising NAC; or
ii) L-carnitine or a salt thereof, or a dipeptide or a salt thereof, comprising L-carnitine; or acetyl-L-carnitine (ALCAR) or a salt thereof or a dipeptide or salt thereof, or a tripeptide or salt thereof, comprising ALCAR;
wherein:
i) at least one amino acid entity of (a)-(c) is not provided as a peptide of more than 20 amino acid residues in length;
ii) the total weight (wt.) % of (a)-(d) is greater than the total wt. % of non-amino acid entity protein components or other amino acid entity components in the composition on a dry weight basis; and
iii) two or more (e.g., all) of phenylalanine, tyrosine, or glutamine is absent from the composition, or if present, is present at less than 1 wt. % of the total wt. of the composition on a dry weight basis.

31. The method, combination, or composition of any preceding claim, wherein two, three, four, five, six, seven, or eight amino acid entities of (a)-(b); (a)-(d); or (a)-(e) is not provided as a peptide of more than 20 amino acid residues in length.

32. The method, combination, or composition of any preceding claim, wherein the composition does not comprise a peptide of more than 20 amino acid residues in length, or if a peptide of more than 20 amino acid residues in length is present, the peptide is present at less than 10 wt. % of the total wt. of amino acid entities in the composition (in dry form).

33. The method, combination, or composition of any preceding claim, wherein two, three, four, five, six, seven, or eight amino acid entities in (a)-(b); (a)-(d); or (a)-(e) are in one or both of free amino acid form or salt amino acid form in the composition (in dry form).

34. The method, combination, or composition of claim 33, wherein at least 35 wt. % of the total wt. of the composition (in dry form) is two, three, four, five, six, seven, eight, nine, ten, or more (e.g., all) amino acid entities in (a)-(b); (a)-(d); or (a)-(e) in one or both of free amino acid form or salt amino acid form.

35. The method or composition of any of claims 26-28 or 30-34, wherein:
i) the wt. % of the BCAA entities is at least 37 wt. % of the total wt. of amino acid entities in the composition (in dry form);
ii) the wt. % of the UCAA entities is at least 25 wt. % of the total wt. of amino acid entities in the composition (in dry form); or
iii) the wt. % of the EAA entities is at least 16 wt. % of the total wt. of amino acid entities in the composition (in dry form).

36. The method or composition of any of claims 26-28 or 30-35, wherein a wt. ratio of the BCAA entity or BCAA entities : the UCAA entity or UCAA entities : the EAA entity or EAA entities in 20+/- 15% : 15 +/- 15%: 9+/- 15%, where the ratios are determined based on an equivalent amount of each amino acid in free form.

37. The method or composition of any of claims 26-28 or 30-36, wherein three, four, five, six, seven, or eight amino acid entities in (a)-(c) is selected from Table 1.

38. The method or composition of any of claims 26-28 or 30-37, wherein the composition comprises:
a) the leucine amino acid entity is chosen from:
i) L-leucine or a salt thereof,
ii) a dipeptide or salt thereof, or tripeptide or salt thereof, comprising L-leucine, or
iii) β-hydroxy-β-methylbutyrate (HMB) or a salt thereof;
b) one or both of:
i) the ornithine amino acid entity is L-ornithine or a salt thereof or a dipeptide or salt thereof, or tripeptide or salt thereof, comprising L-ornithine; or
ii) the aspartate amino acid entity is L-aspartate or a salt thereof or a dipeptide or salt thereof, or tripeptide or salt thereof, comprising L-aspartate;
c) the EAA entity is chosen from:
i) L-histidine or a salt thereof,
ii) a dipeptide or salt thereof, or tripeptide or salt thereof, comprising L-histidine,
iii) L-lysine or a salt thereof,
iv) a dipeptide or salt thereof, or tripeptide or salt thereof, comprising L-lysine,
v) L-threonine or a salt thereof, or
vi) a dipeptide or salt thereof, or tripeptide or salt thereof, comprising L-threonine; and
d) one or two of:
i) N-acetylcysteine (NAC) or a salt thereof, or a dipeptide or a salt thereof, comprising NAC; or
ii) L-carnitine or a salt thereof, or a dipeptide or a salt thereof, comprising L-carnitine; or acetyl-L-carnitine (ALCAR) or a salt thereof or a dipeptide or salt thereof, or a tripeptide or salt thereof, comprising ALCAR.

39. The method or composition of claim 38, wherein the composition further comprises one or both of an isoleucine amino acid entity or a valine amino acid entity.

40. The method or composition of claim 39, wherein the isoleucine amino acid-entity is L-isoleucine or a salt thereof or a dipeptide or salt thereof, or tripeptide or salt thereof, comprising L-isoleucine.

41. The method or composition of either of claims 39 or 40 , wherein the valine amino acid entity is L-valine or a salt thereof or a dipeptide or salt thereof, or tripeptide or salt thereof, comprising L-valine.

42. The method or composition of any of claims 39-41, wherein a wt. ratio of the leucine amino acid entity : the isoleucine amino acid entity : the valine amino acid entity : the ornithine amino acid entity : the aspartate amino acid entity : the histidine amino acid entity : the threonine amino acid entity : the lysine amino acid entity is 8+/- 20% : 4+/- 20% : 8 +/- 20% : 7.5+/- 20% : 7.5+/- 20% : 3+/- 20% : 3+/- 20% : 3+/- 20%, where the ratios are determined based on an equivalent amount of each amino acid in free form.

43. The method or composition of any of claims 26-28 or 30-42 , wherein the composition comprises: L-leucine or a salt thereof, L-isoleucine or a salt thereof, L-valine or a salt thereof, L-ornithine or a salt thereof, L-aspartate or a salt thereof, L-histidine or a salt thereof, L-threonine or a salt thereof, L-lysine or a salt thereof, and one or two of:
i) NAC or a salt thereof; or
ii) L-carnitine or a salt thereof, or ALCAR or a salt thereof.

44. The method, combination, or composition of any preceding claim, wherein the composition is formulated with a pharmaceutically acceptable carrier.

45. The method or combination of any of claims 1-22, wherein the therapeutic agent is formulated with a pharmaceutically acceptable carrier.

46. The method, combination, or composition of any preceding claim, wherein the composition is formulated as a dietary composition.

47. The method or composition of any of claims 23-44 or 46, wherein the subject has: cirrhosis, cirrhotic sarcopenia, hepatic insufficiency, End Stage Liver Disease, hepatic encephalopathy, or a combination thereof.

48. The method or composition of any of claims 23-44, 46, or 47, wherein administration of the composition results in at least one of the following: a) increased level of BCAAs; b) decreased level of aromatic amino acids (AAAs); c) decreased level of ammonia; d) increased level of protein, e.g., increased protein synthesis; e) increased activation of mTORC 1; f) decreased level of myostatin; g) decreased level of creatinine; h) increased level of albumin; i) decreased level of bilirubin; j) increased Fischer’s ratio (e.g., increased level of BCAAs relative to the level of AAAs); or k) an increased level of valine relative to a level of phenylalanine.

49. The method or composition of any of claims 23-44 or 46-48, wherein the composition is administered with a carbohydrate supplement, e.g., when administered in the night, late evening, or before bedtime.

50. The method or combination of any of claims 1-22, 31-34, or 45, wherein the subject has a fatty liver disease or disorder, e.g., the fatty liver disease or disorder is chosen from:
non-alcoholic fatty liver disease (NAFLD), e.g., non-alcoholic steatohepatitis (NASH), non-alcoholic fatty liver (NAFL), or pediatric NAFLD; or
alcoholic fatty liver disease (AFLD), e.g., alcoholic steatohepatitis (ASH).

51. The method or combination of any of claims 1-22, 31-34, 45, or 50, wherein the subject has one, two, three, four, five, or more (e.g., all) of cirrhosis, fibrosis, hepatocarcinoma, steatosis, an increased risk of liver failure, an increased risk of death, type 2 diabetes, metabolic syndrome, a high BMI, obesity, gut leakiness, gut dysbiosis, or gut microbiome disturbance.

52. The method or combination of any of claims 1-22, 31-34, 45, 50, or 51, wherein administration of the combination results in one, two, three, four, five, six, seven, eight, or more (e.g., all) of: decreasing or preventing liver fibrosis, decreasing or preventing liver injury, decreasing or preventing hepatocyte inflammation, improving glucose tolerance; improving insulin resistance, decreasing or preventing steatosis, decreasing or preventing hepatocyte ballooning, increasing liver fatty acid oxidation, or improving gut function.

53. The method or combination of any of claims 1-22, 31-34, 45, or 50-52, wherein administering the combination comprises administering the composition and the therapeutic agent together, e.g., as a unit dose that contains both the composition and the therapeutic agent.

54. The method or combination of any of claims 1-22, 31-34, 45, or 50-52, wherein administering the combination comprises administering the composition and the therapeutic agent separately, e.g., as unit dose containing the composition and a unit dose containing the therapeutic agent.
